(19) United States
(12) Patent Application Publication
(10) Pub. No.: US 2016/0186270 A1
(43) Pub. Date: Jun. 30, 2016

(54) SIGNATURE OF CYCLING HYPOXIA AND USE THEREOF FOR THE PROGNOSIS OF CANCER

(71) Applicant: UNIVERSITÉ CATHOLIQUE DE LOUVAIN, Louvain la Neuve (BE)

(72) Inventors: Olivier FERON, Wezembeek-Oppem (BE); Romain BOIDOT, Arceau (FR); Samuel BRANDERS, Mouscron (BE); Pierre DUPONT, Watermael-Boitsfort (BE); Thibault HELLEPUTTE, Wavre (BE)

(21) Appl. No.: 14/908,825

(22) PCT Filed: Aug. 1, 2014
(86) PCT No.: PCT/EP2014/066643

§ 371 Date: Jan. 29, 2016

(30) Foreign Application Priority Data
Aug. 2, 2013 (EP) 13179071.9

(51) Int. Cl. C12Q 1/68

(52) U.S. Cl. CPC C12Q 1/6886; C12Q 2600/118; C12Q 2600/158; C12Q 2600/106

(57) ABSTRACT

The present invention relates to a signature comprising at least 2 cycling hypoxia markers. The present invention also relates to a non-invasive method for the prognosis of cancer in a subject, wherein said method comprises assessing the expression of markers of a signature of the invention in a sample from said subject; and to a kit for implementing this non-invasive method.

### FIELD OF INVENTION

The present invention relates to the field of cancer prognosis. More specifically, the present invention relates to a signature based on differential gene expression in conditions of cycling hypoxia, for the prognosis of cancer in a subject.

### BACKGROUND OF INVENTION

Cancer is a general term referring to a broad group of diseases characterized by unregulated and uncontrolled cell growth and division. These diseases caused in 2007 about 8 million death worldwide, and are currently the second leading cause of death in developed countries. As prognostic and response to treatments are subject-dependent, there is a need for prognostic and/or predictive means, allowing estimating for each subject the progression of his/her disease and/or his/her response to a given treatment.

Several prognostic or predictive means are currently known in the prior art. Among them, some correspond to a signature, i.e. are based on specific gene expression of tumors or peritumoral tissues.

For example, the European patent application EP 1 754 795 describes a method for predicting relapse of breast cancer in bone by analyzing the expression of a group of 76 genes. This prognostic signature is known in the art as the Gene76 signature.

Moreover, the international patent application WO 02/103320 describes genetic markers whose expression is correlated with breast cancer. More specifically, this patent application describes a genetic signature comprising 70 genes, known as Gene70 or Mammaprint, for the diagnosis and the prognosis of breast cancer in a subject.

Furthermore, the international patent application WO2006/052862 describes a signature useful for predicting whether cancer patients are likely to have a beneficial response to treatment with chemotherapy. The specific signature disclosed by WO2006/052862 corresponds to the Oncotype DX signature developed for breast cancer patients.

Both Oncotype DX signature and Mammaprint signature are approved for clinical use.

However, the signatures of the prior art present the drawback to be designed for one type of cancer only. For example, the above cited signatures were developed for breast cancer. There is thus a need for a genetic signature that may be used for the prognosis of not only one cancer type, but of several cancers. Especially, there is a need for a signature that may be used for the prognosis of all tumors.

A common characteristic of tumors is cycling hypoxia. Cycling hypoxia corresponds to a temporal instability in oxygen transport, as a result of instabilities in microvessel red blood cell flux within tumors. Indeed, tumor angiogenesis and glycolytic metabolism are two responses of cancer cells to a deficit in oxygen. The building of new blood vessels to bring O₂ and the uncoupling from mitochondrial oxidative phosphorylation to survive under low O₂ are actually two complementary responses to hypoxia. These somehow opposite modes of adaptation account for local and temporal heterogeneities in tumor O₂ distribution. The extent of cycling hypoxia may reflect tumor plasticity and thus may be a mark of the capacity of tumor cells to survive and proliferate in a hostile environment.

The inventors herein showed that cycling hypoxia has the potential to lead to common alterations in the expression of some transcripts. They thus developed a signature of cycling hypoxia of particular clinical relevance for the prognosis of cancers.

### SUMMARY

The present invention thus relates to a signature comprising at least 2 cycling hypoxia markers. In one embodiment, the signature comprises at least 3, preferably at least 5, more preferably at least 10 cycling hypoxia markers. In one embodiment, said cycling hypoxia markers are selected from the list of 1379 cycling hypoxia markers of Table 1, fragments, variants and equivalents thereof. In another embodiment, said cycling hypoxia markers are selected from the list of 651 cycling hypoxia markers of Table 2, fragments, variants and equivalents thereof. In another embodiment, said cycling hypoxia markers are selected from the list of 298 cycling hypoxia markers of Table 3, fragments, variants and equivalents thereof. In another embodiment, said cycling hypoxia markers are selected from the list of 167 cycling hypoxia markers of Table 4, fragments, variants and equivalents thereof. In another embodiment, said cycling hypoxia markers are selected from the list of 96 cycling hypoxia markers of Table 5, fragments, variants and equivalents thereof. In another embodiment, said cycling hypoxia markers are selected from the list of 74 cycling hypoxia markers of Table 6, fragments, variants and equivalents thereof. In another embodiment, said cycling hypoxia markers are selected from the list of 37 cycling hypoxia markers of Table 7, fragments, variants and equivalents thereof. In another embodiment, said cycling hypoxia markers are selected from the list of 10 cycling hypoxia markers of Table 8, fragments, variants and equivalents thereof. In another embodiment, said signature comprises the 10 cycling hypoxia markers of Table 8, variants, fragments and equivalents thereof.

The present invention also relates to a non-invasive method for the prognosis of cancer in a subject, or for predicting the response of a subject to a specific treatment, wherein said method comprises assessing the expression of markers of a signature as described hereinabove in a sample from said subject. Therefore, the present invention also relates to a non-invasive method for the prognosis of cancer in a subject, or for predicting the response of a subject to a specific treatment, wherein said method comprises assessing the expression of markers of a signature comprising at least 2 cycling hypoxia markers in a sample from said subject. In one embodiment, the signature comprises at least 3, preferably at least 5, more preferably at least 10 cycling hypoxia markers. In one embodiment, the cycling hypoxia markers are selected from the list of 1379 cycling hypoxia markers of Table 1, fragments, variants and equivalents thereof. In one embodiment, the cycling hypoxia markers are selected from the list of 651 cycling hypoxia markers of Table 2, fragments, variants and equivalents thereof. In one embodiment, the cycling hypoxia markers are selected from the list of 298 cycling hypoxia markers of Table 3, fragments, variants and equivalents thereof. In one embodiment, the cycling hypoxia markers are selected from the list of 167 cycling hypoxia markers of Table 4, fragments, variants and equivalents thereof. In one embodiment, the cycling hypoxia markers are selected from the list of 96 cycling hypoxia markers of Table 5, fragments, variants and equivalents thereof. In one embodiment, the cycling hypoxia markers are selected from the list of 74 cycling hypoxia markers of Table 6, fragments, variants and equivalents thereof. In one embodiment, the cycling hypoxia markers are selected from the list of 37 cycling hypoxia markers of Table 7, fragments, variants and equivalents thereof. In one embodiment, the cycling hypoxia markers are selected from the list of 10 cycling hypoxia markers of Table 8, fragments, variants and equivalents thereof. In one embodiment, the signature comprises the 10 cycling hypoxia markers of Table 8, variants, fragments and equivalents thereof.

In one embodiment, said method comprises mathematically combining the expression profile of markers in a score. In one embodiment, said sample is a biopsy sample or a bodily fluid sample of said subject. In one embodiment, the method of the invention further comprises comparing said expression with a reference expression profile.

The present invention further relates to a kit for determining the expression profile of a genetic signature as described hereinabove, or for implementing the non-invasive method as described hereinabove, wherein said kit comprises means for determining the expression of the cycling hypoxia markers of the signature of the invention. In one embodiment, said means for determining the expression of the markers of the signature is a microarray comprising probes specific for said cycling hypoxia markers. In another embodiment, said means for determining the expression of the cycling hypoxia markers are qPCR primers specific for said cycling hypoxia markers.

### DEFINITIONS

In the present invention, the following terms have the following meanings:
“Prognosis” refers to the likelihood of cancer-attributable death or cancer progression, including recurrence and metastatic spread of a neoplastic disease, during the natural history of the disease, or to the likelihood of a beneficial response to a specific treatment, wherein a beneficial response means an improvement in any measure of patient status including, but not limited to, overall survival, long-term survival (i.e. survival for at least 3, preferably at least 5, 8, or 10 years following diagnosis, surgery or other treatment), recurrence-free survival, and distant recurrence-free survival. Accordingly, a “prognostic signature” refers to a signature that may be used for the prognosis of a subject. In one embodiment, the term “prognostic signature” also includes “predictive signature”, wherein said term refers to a signature that may be used for anticipating the response of a subject to a specific treatment.
“Normoxia” refers to an oxygen tension condition corresponding to healthy tissues. In one embodiment, in conditions of in vitro cell culture, normoxia may refer to a condition with a concentration of O₂ ranging from about 10 to about 21%, preferably from about 15 to about 21%, and more preferably of about 20-21% O₂.
“Hypoxia” refers to a condition wherein the oxygen tension is inferior to the oxygen tension of healthy tissues. In one embodiment, in conditions of in vitro cell culture, hypoxia may refer to a condition with at most 5% O₂, preferably to a condition with about 1% O₂.
“Cycling hypoxia” (also known as “cyclic hypoxia”) refers to a temporal instability in oxygen transport. Cycling hypoxia thus corresponds to alternating normoxia and hypoxia cycles.
“Signature” refers to a group of markers (i.e. at least 2, preferably at least 3, more preferably at least 5, and even more preferably at least 10 markers) whose combined expression profile is indicative of a biological condition (such as, for example, cycling hypoxia), or of a particular prognosis or of a particular response of a subject to a treatment.
A “marker” corresponds to a nucleotide sequence isolated from the genome, preferably to a gene in the genome, i.e. each marker is identifiable as all or a portion of a gene. A marker may thus correspond to an entire gene, or to an EST (wherein EST stands for Expressed Sequence Tag) derived from this gene.
“Expression” refers interchangeably to expression of a marker, including the encoded polypeptide or protein. Expression of a marker may be determined, for example, by immunoassay using one or more antibody(ies) that bind(s) with the polypeptide. Alternatively, expression of a marker may be determined by measurement of mRNA levels, for example, by RT-PCR, RT-qPCR (wherein qPCR stands for quantitative PCR), or using a microarray, or using sequencing methods. In one embodiment, the term “expression” of a marker may also refer to modification of a protein or peptide, preferably to post-translational modification of a protein or peptide.
“Subject” refers to an animal, preferably a mammal, more preferably a human. In one embodiment, the subject is a patient, i.e. a recipient of health care services. Preferably, the subject is a cancer patient, i.e. he/she was previously diagnosed with cancer.
“About” preceding a figure means plus or less 10% of the value of said figure.

### DETAILED DESCRIPTION

The present invention first relates to a signature of cycling hypoxia, wherein said signature comprises markers whose expression is different between a normoxic condition and a cycling hypoxia condition.

In one embodiment of the invention, the signature of the invention comprises at least 2 markers, preferably at least 3 markers, 4 markers, more preferably at least 5 markers, and even more preferably at least 10 markers.

The present invention thus also relates to a marker whose expression is different between a normoxic condition and a cycling hypoxia condition. A marker whose expression is different between a normoxic condition and a cycling hypoxia condition will be hereinafter referred as a “cycling hypoxia marker”.

Methods for determining cycling hypoxia markers are well-known from the skilled artisan, and include, without limitation, comparing the transcriptome (in an embodiment wherein expression relates to transcription of a marker) or proteome (in an embodiment wherein expression relates to translation of a marker) in a condition of normoxia and in a condition of cycling hypoxia. An example of such a method, based on the comparison of transcriptomes, is presented in the Examples.

Examples of post-translational modifications of a protein or peptide include, but are not limited to, phosphorylation, myristoylation, palmitoylation, isoprenylation, glypiation, lipoylation, O-, N- or S-acylation, alkylation, glycosylation, malonylation, hydroxylation, nucleotide addition, oxidation, sumoylation, ubiquitination, citrullination, deamidation, formation of disulfide bridges, proteolytic cleavage, racemization and the like. Examples of methods for assessing post-translational modifications of a protein or peptide include, but are not limited to, mass spectroscopy, immunoblotting, Eastern blotting, and the like.

In one embodiment of the invention, a marker is considered as differentially expressed in conditions of normoxia and cycling hypoxia if, according to a t-test, the p-value after FDR correction is lower than 0.05, preferably lower than 0.01.

In one embodiment, cycling hypoxia markers are selected from the list of the 1379 cycling hypoxia markers of Table 1 below, as well as their variants, fragments or equivalents. Table 1 comprises cycling hypoxia markers identified in the conditions of the Example and presenting a p-value after FDR correction lower than 0.05.

Pathways refer to the KEGG pathway database (http://www.genome.jp/kegg/).

In the Table 1 below, and in Tables 2-8, probesets are indicated according to the nomenclature of “Human gene 1.0ST”.

TABLE 1

| GeneBank | Name of the | | |
|---|---|---|---|
| Accession Number | marker | Pathways | Probeset |
| NR_002312 | RPPH1 | | 7977507 |
| BC018448 | MALAT1 | | 7949410 |
| NR_003287 | RN28S1 | | 7942875, |
| | | | 8059576, |
| | | | 7917645, |
| | | | 7942791 or |
| | | | 8151234 |
| AF284753 | UIMC1 | | 7911343 or |
| | | | 8165703 |
| NM_014248 | RBX1 | 03420, 04110, | 8073334 |
| | | 04114, 04120, | |
| | | 04141, 04310, | |
| | | 04350, 04710, | |
| | | 05200, 05211 | |
| NM_177987 | TUBB8 | 04145, 04540, | 7911355 |
| | | 05130 | |
| NM_170601 | SIAE | | 7944867 |
| NM_001012708 | KRTAP5-3 | | 7945652 |
| NR_029710 | MIR193A | | 8006321 |
| NM_000981 | RPL19 | 3010 | 8006845 |
| NM_012217 | TPSD1 | | 7992191 |
| NM_003792 | EDF1 | | 8165309 |
| BC013044 | DNAJA2 | 4141 | 7995379 |
| NR_029824 | MIR128-2 | | 8078527 |
| NM_004352 | CBLN1 | | 8001329 |
| NM_001017 | RPS13 | 3010 | 7946812 |
| NM_001037160 | CYS1 | | 8050232 |
| NM_003731 | SSNA1 | | 8159609 |
| NM_006160 | NEUROD2 | | 8014865 |
| NM_001417 | EIF4B | 03013, 04150 | 7963575 |
| NM_017854 | TMEM160 | | 8037853 |
| NM_016057 | COPZ1 | | 7955896 |
| NM_152568 | NKX6-3 | | 8150433 |
| NM_016170 | TLX2 | | 8042896 |
| NR_002715 | RN7SL1 | | 8040338 |
| NM_016564 | CEND1 | | 7945536 |
| AK302042 | LOC440518 | | 8027343 |
| NM_014206 | C11orf10 | | 7948606 |
| NR_033335 | SNORA70G | | 7964830 |
| NM_003094 | SNRPE | 3040 | 8160033 or |
| | | | 7908988 |
| NM_012322 | LSM5 | 03018, 03040 | 8138912 |
| NM_145232 | CTU1 | 4122 | 8038782 |
| NR_029583 | MIR197 | | 7903717 |
| NM_032231 | FAM96A | | 7989611 |
| NR_024583 | POM121L8P | | 8071168 |
| NM_007241 | SNF8 | 4144 | 8016508 |
| NM_000307 | POU3F4 | | 8168567 |
| NM_013299 | SAC3D1 | | 7941122 |
| NM_005608 | PTPRCAP | | 7949792 |
| NM_006327 | TIMM23 | | 7927548 |
| NM_016424 | LUC7L3 | | 8016733 |
| NM_144615 | TMIGD2 | | 8032782 |
| NM_001135086 | PRSS41 | | 7992716 |
| NM_003512 | HIST1H2AC | 5322 | 8117372 |
| NM_000863 | HTR1B | 4080 | 8127692 |
| NM_145203 | CSNK1A1L | 04310, 04340 | 7971071 |
| NR_000009 | SNORD4B | | 8005957 |
| NM_001080113 | C14orf184 | | 7980859 |
| AK123383 | LOC642648 | | 8076747 |
| NM_032479 | MRPL36 | | 8110861 or |
| | | | 8180305 |
| NM_031210 | SLIRP | | 7975989 |
| NM_023002 | HAPLN4 | | 8035646 |
| NM_182532 | TMEM61 | | 7901687 |
| NM_003538 | HIST1H4A | 5322 | 8117334 |
| AK125166 | LOC441268 | | 8141166 |
| NM_001001521 | UGP2 | 00040, 00052, | 8052624 |
| | | 00500, 00520, | |
| | | 01100 | |
| NR_001445 | RN7SK | | 8120249 |
| NM_001551 | IGBP1 | | 8168087 |
| NM_138417 | KTI12 | | 7916130 |
| NM_031213 | FAM108A1 | | 8032371, |
| | | | 7904869, |
| | | | 7904948, |
| | | | 7924230 or |
| | | | 8074842 |
| BC001181 | FAM173A | | 7992043 |
| NM_001031 | RPS28 | 3010 | 8005471, |
| | | | 8025395 or |
| | | | 7942824 |
| NM_004175 | SNRPD3 | 03040, 05322 | 8071920 |
| NM_001044370 | MPPED1 | | 8073623 |
| BC005079 | C2orf42 | | 8052834 |
| NM_003542 | HIST1H4C | 5322 | 8117368 |
| BC033986 | LOC440934 | | 8048712 |
| NM_001082575 | RBFOX3 | | 8018993 |
| NM_017900 | AURKAIP1 | | 7911532 or |
| | | | 8039923 |
| NM_001024598 | HES3 | | 7897280 |
| NM_022061 | MRPL17 | | 7946267 |
| NM_001029 | RPS26 | 3010 | 8007797 or |
| | | | 8154363 |
| NM_016060 | MED31 | | 8011968 |
| NM_012394 | PFDN2 | | 7921786 |
| NM_015965 | NDUFA13 | 00190, 05010, | 8027205 |
| | | 05012, 05016 | |
| NM_080603 | ZSWIM1 | | 8063074 |
| NM_021104 | RPL41 | 3010 | 7957530, |
| | | | 7965467, |
| | | | 7982129, |
| | | | 8105432, |
| | | | 8075691 or |
| | | | 8061364 |
| NM_000847 | GSTA3 | 00480, 00980, | 8127087 |
| | | 00982 | |
| NM_032753 | RAX2 | | 8032601 |
| NM_003684 | MKNK1 | 04010, 04910 | 7915846 |
| NM_003577 | UTF1 | | 7931553 |
| NM_022363 | LHX5 | | 7966631 |
| NM_001037495 | DYNLL1 | 4962 | 7967067 or |
| | | | 7959164 |
| NM_004609 | TCF15 | | 8064370 |
| AK098732 | TRAP1 | | 7992954 |
| NM_144999 | LRRC45 | | 8010719 |
| NM_001018138 | NME2 | 00230, 00240, | 8180388, |
| | | 01100 | 8180389, |
| | | | 8180387 or |
| | | | 8180386 |
| NM_002528 | NTHL1 | 3410 | 7998692 |
| NM_006087 | TUBB4 | 04145, 04540, | 8025051 |
| | | 05130 | |
| NM_003493 | HIST3H3 | 5322 | 7924884 |
| NR_026800 | KIAA0125 | | 7977440 |
| NM_015456 | COBRA1 | | 8159654 |
| NM_006088 | TUBB2C | 04145, 04540, | 8165496 |
| | | 05130 | |
| NM_002307 | LGALS7 | | 8036584 or |
| | | | 8028546 |
| NM_181887 | UBE2D3 | 04120, 04141 | 8180330, |
| | | | 8180335, |
| | | | 8180334, |
| | | | 8180331, |
| | | | 8180333, |
| | | | 8180329, |
| | | | 8180332 or |
| | | | 8102024 |
| NM_001348 | DAPK3 | 05200, 05219 | 8032718 |
| NM_005319 | HIST1H1C | | 8124397 |
| NM_178536 | LCN12 | | 8159501 |
| NR_003666 | SPDYE7P | | 8133209 |
| AK125308 | LOC100129484 | | 8137962 |
| NM_020412 | CHMP1B | 4144 | 8020179 |
| NM_003550 | MAD1L1 | 04110, 04914 | 8137805 |
| NM_032527 | ZGPAT | | 8064156 |
| NR_003051 | RMRP | | 8161024 |
| NR_029681 | MIR140 | | 7997008 |
| NM_006858 | TMED1 | | 8034101 |
| NM_006312 | NCOR2 | 4330 | 7959772 |
| AK095987 | FLJ38668 | | 8054449 |
| ENST00000427835 | C20orf61 | | 8065013 |
| NM_001144936 | C11orf95 | | 7949015 |
| NM_173547 | TRIM65 | | 8018502 |
| NM_014370 | SRPK3 | | 8170753 |
| NM_005574 | LMO2 | | 7947450 |
| NM_001007595 | C2CD4B | | 7989473 |
| NM_001168 | BIRC5 | 05200, 05210 | 8018860 |
| NM_021012 | KCNJ12 | | 8005726 |
| NM_144589 | COMTD1 | | 7934544 |
| NM_016589 | TIMMDC1 | | 8081867 |
| NM_012315 | KLK9 | | 8038716 |
| NM_006292 | TSG101 | 4144 | 7947015 |
| NM_033055 | HIAT1 | | 7903294 |
| NM_001113201 | NACA | | 7964262 |
| NM_181838 | UBE2D2 | 04120, 04141, | 8108435 |
| | | 05131 | |
| NM_005973 | PRCC | | 7906235 |
| NM_005274 | GNG5 | 4062 | 8174509 |
| NM_006770 | MARCO | 4145 | 8044773 |
| NM_014674 | EDEM1 | 4141 | 8085116 |
| NM_145657 | GSX1 | | 7968260 |
| NM_002003 | FCN1 | | 8165011 |
| NM_003001 | SDHC | 00020, 00190, | 8011212 |
| | | 01100, 05010, | |
| | | 05012, 05016 | |
| NM_018942 | HMX1 | | 8104136 |
| NM_006848 | CCDC85B | | 7941457 |
| NM_032338 | LLPH | | 7956876 |
| NM_015971 | MRPS7 | | 8009784 |
| NM_020180 | CELF4 | | 8022952 |
| NM_001080495 | TNRC18 | | 8137959 |
| NM_006181 | NTN3 | 4360 | 7992632 |
| AK094921 | LOC100131763 | | 8049950 |
| NM_198545 | C1orf187 | | 7897737 |
| NM_002066 | GML | | 8148565 |
| NM_031899 | GORASP1 | | 8086317 |
| NM_012452 | TNFRSF13B | 04060, 04672, | 8013061 |
| | | 05340 | |
| NM_138574 | HDGFL1 | | 8117172 |
| NM_024816 | RABEP2 | | 8000616 |
| NM_022097 | CHP2 | 04010, 04020, | 7994123 |
| | | 04114, 04210, | |
| | | 04310, 04360, | |
| | | 04370, 04650, | |
| | | 04660, 04662, | |
| | | 04720, 05010, | |
| | | 05014 | |
| NM_006801 | KDELR1 | 5110 | 8038078 |
| NM_004939 | DDX1 | | 8040386 |
| NM_130784 | SYCE1 | | 7937247 |
| NM_019082 | DDX56 | | 8139392 |
| NM_001039916 | ZNF384 | | 7953390 |
| NM_016602 | CCR10 | 04060, 04062, | 8015681 |
| | | 04672 | |
| NR_024591 | POM121L1P | | 8074714 or |
| | | | 8074867 |
| NM_020064 | BARHL1 | | 8158912 |
| NM_006356 | ATP5H | 00190, 01100, | 8018288 |
| | | 05010, 05012, | |
| | | 05016 | |
| NM_012249 | RHOQ | 4910 | 8041808 |
| NR_002951 | SNORA2B | | 7962829 |
| NR_004430 | RNU1-1 | | 7919269, |
| | | | 7919349, |
| | | | 7898375, |
| | | | 7898411, |
| | | | 7912800, |
| | | | 7912850, |
| | | | 7919576, |
| | | | 7973896 or |
| | | | 7978568 |
| NM_001126128 | PROK2 | | 8088813 |
| NM_016063 | HDDC2 | | 8129363 |
| NM_005706 | TSSC4 | | 7937813 |
| NR_002781 | TSPY26P | | 8065603 |
| NM_175064 | SPDYE1 | | 8140424, |
| | | | 8140454 or |
| | | | 8132531 |
| NM_138350 | THAP3 | | 7897329 |
| AY730278 | CENPVL1 | | 8167652 or |
| | | | 8172715 |
| NM_002669 | PLRG1 | 3040 | 8103289 |
| NM_006476 | ATP5L | 00190, 01100 | 7944216 |
| NM_031909 | C1QTNF4 | | 7947928 |
| NM_032805 | ZSCAN10 | | 7998921 |
| NM_001804 | CDX1 | | 8109226 |
| NM_014976 | PDCD11 | | 7936096 |
| AK097604 | LOC100130285 | | 7998265 |
| NM_003168 | SUPT4H1 | | 8016982 |
| NM_016835 | MAPT | 04010, 05010 | 8016263 |
| NM_001002 | RPLP0 | 3010 | 8109750 or |
| | | | 7966996 |
| NM_016305 | SS18L2 | | 8079074 |
| NM_001033113 | ENTPD8 | 00230, 00240 | 8165538 |
| NM_003926 | MBD3 | | 8032275 |
| NM_031280 | MRPS15 | | 7914940 |
| NR_026676 | RPS2P32 | | 8131869 |
| NM_145803 | TRAF6 | 04010, 04120, | 7947540 |
| | | 04144, 04380, | |
| | | 04620, 04621, | |
| | | 04622, 04722, | |
| | | 05140, 05142, | |
| | | 05145, 05160, | |
| | | 05200, 05222, | |
| NM_000858 | GUK1 | 00230, 01100 | 7910241 |
| NM_139172 | TMEM190 | | 8031475 |
| NM_018047 | RBM22 | 3040 | 8115168 |
| NM_182702 | PRSS42 | | 8086683 |
| NM_003859 | DPM1 | 00510, 01100 | 8067017 |
| NM_003002 | SDHD | 00020, 00190, | 7943853 or |
| | | 01100, 05010, | 7899016 |
| | | 05012, 05016, | |
| NR_026716 | KIR3DX1 | | 8031200 |
| NM_015719 | COL5A3 | 04510, 04512, | 8033825 |
| | | 04974, 05146 | |
| L20860 | SEPT5-GP1BB | | 8071272 |
| NM_101395 | DYRK1A | | 8068551 |
| NM_014419 | DKKL1 | | 8030292 |
| NR_026557 | PLK5 | | 8024331 |
| BC043386 | C19orf68 | | 8029996 |
| NM_001080440 | OTOL1 | | 8083770 |
| NM_144578 | MAPK1IP1L | | 7974455 |
| NM_012145 | DTYMK | 00240, 01100 | 8060286 or |
| | | | 8077262 |
| NM_002804 | PSMC3 | 3050 | 7947867 |
| NM_001172743 | RAI2 | | 8171539 |
| NM_016547 | SDF4 | | 7911422 |
| NM_175741 | C15orf55 | | 7982516 |
| NM_003910 | BUD31 | 3040 | 8134589 |
| NM_014342 | MTCH2 | | 7947934 |
| NM_001013 | RPS9 | 3010 | 8180398 or |
| | | | 8031152 |
| NM_021646 | ZNF500 | | 7999196 |
| AY341951 | FAM138D | | 7960172 |
| NM_005034 | POLR2K | 00230, 00240, | 8147654 |
| | | 01100, 03020, | |
| | | 05016 | |
| NM_001005922 | KRTAP5-1 | | 7945645 |
| NM_001105669 | TTC24 | | 7906177 |
| NM_006043 | HS3ST2 | 534 | 7994052 |
| NM_173641 | EPHA10 | | 7915078 |
| NM_001010908 | C1QL3 | | 7932308 |
| NM_001164094 | COPS7A | | 7953395 |
| NM_014582 | OBP2A | | 8180231 |
| NM_024319 | C1orf35 | | 7924842 |
| NM_003375 | VDAC2 | 04020, 05012, | 8042335 or |
| | | 05016 | 7928524 |
| NM_007374 | SIX6 | | 7974793 |
| NM_001003684 | UQCR10 | 00190, 01100, | 8072274 |
| | | 04260, 05010, | |
| | | 05012, 05016 | |
| NR_000025 | SNORD15B | | 7942594 |
| AF304442 | C21orf118 | | 8068046 |
| NM_016312 | WBP11 | 3040 | 7961489 |
| NM_080875 | MIB2 | | 7896985 |
| NM_005922 | MAP3K4 | 04010, 04912 | 8130624 |
| NR_002576 | SNORA21 | | 8014755 |
| NM_018462 | BRK1 | 4810 | 8085287 |
| NR_003013 | SCARNA16 | | 8010137 |
| NM_005609 | PYGM | 00500, 04910 | 7949124 |
| NM_016438 | HIGD1B | | 8007701 |
| NM_004855 | PIGB | 00563, 01100 | 7983811 |
| NR_026713 | FAM182A | | 8065527 or |
| | | | 8061490 |
| NM_003537 | HIST1H3B | 5322 | 8124388 |
| NM_001040436 | YARS2 | 970 | 7962203 |
| NM_014171 | CRIPT | | 8041813 |
| NM_053056 | CCND1 | 04110, 04115, | 7950012 |
| | | 04310, 04510, | |
| | | 04630, 05200, | |
| | | 05210, 05212, | |
| | | 05213, 05214, | |
| | | 05215, 05216, | |
| | | 05218, 05219, | |
| | | 05220, 05221, | |
| | | 05222, 05223, | |
| | | 05416 | |
| AF067420 | IGHA1 | | 7995263 or |
| | | | 7981722 |
| NM_198180 | QRFP | | 8164630 |
| BC004224 | SHANK2-AS3 | | 7942228 |
| NM_019107 | C19orf10 | | 8032863 |
| NM_001800 | CDKN2D | 4110 | 8034075 |
| NM_014860 | SUPT7L | | 8051204 |
| NM_000183 | HADHB | 00062, 00071, | 8040639 |
| | | 00280, 01100 | |
| NM_001258 | CDK3 | | 8010021 |
| NM_005247 | FGF3 | 04010, 04810, | 7950036 |
| | | 05200, 05218 | |
| NM_007255 | B4GALT7 | 00532, 00534, | 8110399 |
| | | 01100 | |
| NM_016199 | LSM7 | 03018, 03040 | 8032480 |
| BC004943 | MGC10814 | | 8035551 |
| NR_024593 | POM121L10P | | 8075024 |
| NM_145719 | TIGD3 | | 7941255 |
| NM_001164440 | ANKRD33B | | 8104499 |
| NM_174923 | CCDC107 | | 8155073 |
| NM_015276 | USP22 | | 8013486 |
| NM_001164447 | FAM90A10 | | 8144448 |
| NM_133261 | GIPC3 | | 8024676 |
| NM_001037984 | SLC38A10 | | 8019149 |
| NM_006855 | KDELR3 | 5110 | 8073015 |
| NM_021944 | C14orf93 | | 7977868 |
| NM_005430 | WNT1 | 04310, 04340, | 7955170 |
| | | 04916, 05200, | |
| | | 05217 | |
| NM_199044 | NSUN4 | | 7901219 |
| AK125575 | ZNF425 | | 8143708 |
| NM_032344 | NUDT22 | | 7940884 |
| NM_018090 | NECAP2 | | 7898337 |
| NM_000377 | WAS | 04062, 04520, | 8167334 |
| | | 04666, 04810, | |
| | | 05100, 05130, | |
| | | 05131 | |
| NM_001001410 | C16orf42 | | 7998449 |
| NM_148172 | PEMT | 00564, 01100 | 8013120 |
| NM_001032363 | C1orf151 | | 7898574 |
| NM_002949 | MRPL12 | | 8010664 |
| NM_012094 | PRDX5 | 4146 | 7940996 |
| NR_001555 | GOLGA2P2Y | | 8176910 or |
| | | | 8177413 |
| NM_025072 | PTGES2 | 00590, 01100 | 8164362 |
| NM_138983 | OLIG1 | | 8068235 |
| NM_016568 | RXFP3 | | 8104781 |
| NM_152914 | C17orf103 | | 8013509 |
| NM_014170 | GTPBP8 | | 8081676 |
| NM_003513 | HIST1H2AB | 5322 | 8124391 |
| NM_003278 | CLEC3B | | 8079305 |
| NM_004637 | RAB7A | 04144, 04145, | 8082431 |
| | | 05146 | |
| NM_001144954 | C5orf47 | | 8110068 |
| NM_001048183 | PHACTR4 | | 7899455 |
| NM_004551 | NDUFS3 | 00190, 01100, | 7939825 |
| | | 05010, 05012, | |
| | | 05016 | |
| NM_006808 | SEC61B | 03060, 04141, | 8156838 |
| | | 04145, 05110 | |
| NM_020199 | C5orf15 | | 8114138 |
| NM_030811 | MRPS26 | | 8060599 |
| NR_026581 | MLF2 | | 7960689 |
| NM_002297 | LCN1 | | 8159255 |
| BC104424 | FAHD2B | | 8043682 |
| NM_005714 | KCNK7 | | 7949444 |
| NM_001099435 | SPDYE5 | | 8133654 |
| BC035374 | TCEANC2 | | 7901592 |
| NM_005091 | PGLYRP1 | | 8037742 |
| NM_001013653 | LRRC26 | | 8165453 |
| NM_182498 | ZNF428 | | 8037355 |
| NM_001024678 | LRRC24 | | 8153868 or |
| | | | 8153862 |
| NM_001114600 | C1orf144 | | 7898328 |
| NM_001614 | ACTG1 | 04145, 04510, | 8019183 |
| | | 04520, 04530, | |
| | | 04670, 04810, | |
| | | 05100, 05110, | |
| | | 05130, 05131, | |
| | | 05410, 05412, | |
| | | 05414, 05416 | |
| NM_000383 | AIRE | 04120, 05340 | 8069037 |
| NR_002911 | SNORA71A | | 8066258 |
| NM_001135580 | C19orf71 | | 8024655 |
| NM_145272 | C17orf50 | | 8006569 |
| NM_000154 | GALK1 | 00052, 00520, | 8018428 |
| | | 01100 | |
| AY358234 | LOC100129831 | | 8024444 |
| NM_001100418 | C19orf60 | | 8027032 |
| NM_020996 | FGF6 | 04010, 04810, | 7960407 |
| | | 05200, 05218 | |
| NM_001297 | CNGB1 | 04740, 04744 | 8001615 |
| AK128227 | LOC100130236 | | 7919299 or |
| | | | 7919380 |
| NM_002494 | NDUFC1 | 00190, 01100, | 8102839 |
| | | 05010, 05012, | |
| | | 05016 | |
| NM_198175 | NME1 | 00230, 00240, | 8008517 |
| | | 01100 | |
| NM_000479 | AMH | 04060, 04350 | 8024429 |
| NM_001145250 | SP9 | | 8056825 |
| NR_002144 | LOC407835 | | 8136065 |
| NM_014110 | PPP1R8 | | 7914139 |
| NM_152898 | FERD3L | | 8138450 |
| NR_030170 | MIR202 | | 7937148 |
| NM_177398 | LMX1A | | 7921936 |
| NM_178348 | LCE1A | | 7905528 |
| NM_005444 | RQCD1 | 3018 | 8048340 |
| NM_173806 | PDZD9 | | 8000229 |
| NM_006857 | SNRNP27 | 3040 | 8042495 |
| NM_033644 | FBXW11 | 04114, 04120, | 8115765 |
| | | 04310, 04340, | |
| | | 04710, 05131 | |
| NM_001164456 | FAM90A13 | | 8149204, |
| | | | 8144428, |
| | | | 8144432, |
| | | | 8144436, |
| | | | 8144444 |
| | | | 8144424 or |
| | | | 8144440 |
| NM_020070 | IGLL1 | 5340 | 8074909 |
| NM_005007 | NFKBIL1 | | 8118127, |
| | | | 8177967 or |
| | | | 8179249 |
| NM_177542 | SNRPD2 | 3040 | 8037642 |
| NM_020637 | FGF22 | 04010, 04810, | 8023990 |
| | | 05200, 05218 | |
| NM_144727 | CRYGN | | 8143949 |
| NM_015568 | PPP1R16B | | 8062557 |
| NM_018250 | INTS9 | | 8150014 |
| NM_003517 | HIST2H2AC | 5322 | 7905088 |
| NM_032998 | DEDD | | 7921793 |
| NM_173660 | DOK7 | | 8093807 |
| NM_015679 | TRUB2 | | 8164428 |
| NM_030657 | LIM2 | | 8038815 |
| NM_015918 | POP5 | 03008, 03013 | 7967084 |
| NR_029833 | MIR30C1 | | 7900490 |
| NM_001004 | RPLP2 | 3010 | 7937476 |
| NM_152778 | MFSD8 | 4142 | 8102730 |
| NM_032595 | PPP1R9B | | 8016628 |
| NM_006686 | ACTL7B | | 8163019 |
| AK291454 | UBE2K | 4120 | 8099918 |
| NM_000983 | RPL22 | 3010 | 7911989 |
| NM_053049 | UCN3 | | 7925950 |
| NM_001030047 | KLK3 | 05200, 05215 | 8030753 |
| NR_003502 | ZNRF2P1 | | 8132209 |
| NM_016734 | PAX5 | | 8161211 |
| NM_052945 | TNFRSF13C | 04060, 04672, | 8076387 |
| | | 05340 | |
| NM_006299 | ZNF193 | | 8117655 |
| NM_002764 | PRPS1 | 00030, 00230, | 8169240 |
| | | 01100 | |
| NM_001024675 | C20orf134 | | 8061944 |
| NM_014064 | METTL11A | | 8158544 |
| NM_001002252 | ARL6IP4 | | 7959549 |
| NM_004640 | DDX39B | 03013, 03015, | 8178476, |
| | | 03040 | 8179750 or |
| | | | 8124926 |
| NM_173514 | SLC38A9 | | 8112121 |
| NM_018955 | UBB | 5012 | 8005166 |
| NM_001130861 | CLDN5 | 04514, 04530, | 8074473 |
| | | 04670, 05160 | |
| NM_006432 | NPC2 | 4142 | 7980146 |
| AB016902 | HGC6.3 | | 8130824 |
| NM_000413 | HSD17B1 | 00140, 01100 | 8007263 |
| NM_001142467 | HES4 | | 7911376 |
| NM_181802 | UBE2C | 4120 | 8063043 |
| NM_001164453 | FAM90A20 | | 8144388 |
| NM_178548 | TFAP2E | | 7900001 |
| NM_032810 | ATAD1 | | 7934870 |
| NM_001003682 | TMEM200B | | 7914232 |
| NM_003013 | SFRP2 | 4310 | 8103254 |
| NM_033178 | DUX4 | | 8098732, |
| | | | 8098740, |
| | | | 8098725, |
| | | | 8104122, |
| | | | 8098730, |
| | | | 8098743, |
| | | | 7931665, |
| | | | 7931656, |
| | | | 7931659, |
| | | | 7931662, |
| | | | 7931668 or |
| | | | 7931671 |
| NM_003110 | SP2 | | 8008052 |
| NM_021570 | BARX1 | | 8162472 |
| NR_002798 | NAPSB | | 8038547 |
| NM_001029865 | DBX1 | | 7947129 |
| NM_178138 | LHX3 | | 8165083 |
| NM_005202 | COL8A2 | | 7914880 |
| NM_001069 | TUBB2A | 04145, 04540, | 8116649 or |
| | | 05130 | 8116653 |
| NM_006943 | SOX12 | | 8060334 |
| NR_029485 | MIR15A | | 7971661 |
| NM_000986 | RPL24 | 3010 | 8126450 or |
| | | | 8089249 |
| NM_018158 | SLC4A1AP | | 8041015 |
| NM_012188 | FOXI1 | | 8109901 |
| NM_031917 | ANGPTL6 | | 8033892 |
| NM_006118 | HAX1 | | 7905733 |
| NM_000290 | PGAM2 | 00010, 01100 | 8139276 |
| NM_024888 | LPPR3 | | 8032094 |
| NM_002477 | MYL5 | 04510, 04530, | 8093386 |
| | | 04670, 04810 | |
| NM_138383 | MTSS1L | | 7997135 |
| NM_004435 | ENDOG | 4210 | 8158418 |
| BC008667 | PANK2 | 00770, 01100 | 8060736 |
| NM_174920 | SAMD14 | | 8016615 |
| NM_014581 | OBP2B | | 8180358 |
| NM_176677 | NHLRC4 | | 7991898 |
| NM_031157 | HNRNPA1 | 3040 | 7955890 |
| NR_024420 | LOC389634 | | 7960894 |
| NM_032574 | DPY30 | | 8051387 |
| NM_138705 | CALML6 | 04020, 04070, | 7897026 |
| | | 04114, 04270, | |
| | | 04720, 04722, | |
| | | 04740, 04744, | |
| | | 04910, 04912, | |
| | | 04916, 04970, | |
| | | 04971, 05010, | |
| | | 05214 | |
| BC036197 | MGC39545 | | 7944972 |
| BC036837 | C20orf201 | | 8067812 |
| NM_001111322 | DDX54 | | 7958948 |
| NM_016219 | MAN1B1 | 00510, 01100, | 8159566 |
| | | 04141 | |
| NR_029618 | MIR199A2 | | 7922328 |
| NM_001113324 | TEN1 | | 8010017 |
| NM_015414 | RPL36 | 3010 | 8024966 |
| NM_014183 | DYNLRB1 | | 8062016 |
| NM_004781 | VAMP3 | 04130, 04145 | 7897370 |
| NM_002714 | PPP1R10 | | 8124756, |
| | | | 8178358 or |
| | | | 8179664 |
| NM_020341 | PAK7 | 04012, 04360, | 8064952 |
| | | 04510, 04660, | |
| | | 04810, 05211 | |
| NM_080625 | C20orf160 | | 8061653 |
| NM_003969 | UBE2M | 4120 | 8039805 |
| NM_006232 | POLR2H | 00230, 00240, | 8084488 |
| | | 01100, 03020, | |
| | | 05016 | |
| NM_002900 | RBP3 | | 7933359 |
| NM_025147 | COQ10B | | 8047217 |
| NM_016932 | SIX2 | | 8051949 |
| NM_012210 | TRIM32 | 4120 | 8157516 |
| NM_019612 | IRGC | | 8029318 |
| NM_013234 | EIF3K | | 8028514 |
| NM_178351 | LCE1C | | 7920193 |
| NM_007167 | ZMYM6 | | 7914764 |
| NM_004278 | PIGL | 00563, 01100 | 8005157 |
| NM_014688 | USP6NL | | 7926150 |
| NM_006563 | KLF1 | | 8034578 |
| NM_207305 | FOXD4 | | 8159808 |
| NM_001012710 | KRTAP5-10 | | 7942267 |
| NM_020415 | RETN | | 8025278 |
| NM_005225 | E2F1 | 04110, 05200, | 8065710 |
| | | 05212, 05214, | |
| | | 05215, 05218, | |
| | | 05219, 05220, | |
| | | 05222, 05223 | |
| NM_020839 | WDR48 | | 8078834 |
| NM_003332 | TYROBP | 04380, 04650 | 8036224 |
| NM_003396 | WNT9B | 04310, 04340, | 8007895 |
| | | 04916, 05200, | |
| | | 05217 | |
| NR_026811 | AGSK1 | | 7985571, |
| | | | 7985431, |
| | | | 7990952, |
| | | | 7990902 or |
| | | | 7991714 |
| NM_024095 | ASB8 | | 7962783 |
| NM_001330 | CTF1 | 04060, 04630 | 7994961 |
| NM_080865 | GPR62 | | 8080158 |
| NM_144582 | TEX261 | | 8042566 |
| NM_032328 | EFCAB2 | | 7925585 |
| NM_032772 | ZNF503 | | 7928529 |
| NM_000194 | HPRT1 | 00230, 00983, | 8169984 |
| | | 01100 | |
| NM_001009606 | HS3ST6 | | 7998634 |
| NM_001100119 | XRCC3 | 3440 | 7981447 |
| NR_029703 | MIR150 | | 8038393 |
| NM_057176 | BSND | | 7901691 |
| NR_003008 | SCARNA5 | | 8049297 |
| NM_015913 | TXNDC12 | 480 | 7916120 |
| NM_006612 | KIF1C | | 8004057 |
| NM_002798 | PSMB6 | 3050 | 8003953 |
| NM_000684 | ADRB1 | 04020, 04080, | 7930627 |
| | | 04144, 04540, | |
| | | 04970, 05414 | |
| NM_024812 | BAALC | | 8147756 |
| NM_175078 | KRT77 | | 7963502 |
| NM_144736 | C2orf56 | | 8041495 |
| NM_002460 | IRF4 | | 8116559 |
| NM_001001520 | HDGFRP2 | | 8024864 |
| NM_001168479 | ARMCX5 | | 8168958 |
| NR_029596 | MIR129-1 | | 8135907 |
| NM_005565 | LCP2 | 04380, 04650, | 8115734 |
| | | 04660, 04664 | |
| NM_001099279 | FOXD4L2 | | 8161571, |
| | | | 8155440 or |
| | | | 8161583 |
| NM_198595 | AFAP1 | | 8094030 |
| NM_002676 | PMM1 | 00051, 00520, | 8076355 |
| | | 01100 | |
| NR_003594 | REXO1L2P | | 8151603, |
| | | | 8151607, |
| | | | 8151623, |
| | | | 8151619, |
| | | | 8151627, |
| | | | 8151615 or |
| | | | 8151631 |
| NM_006427 | SIVA1 | | 7977288 |
| NM_032152 | PRAM1 | | 8033587 |
| NM_001144995 | CCDC85C | | 7981273 |
| NR_002449 | SNORA65 | | 8164215 |
| NM_001002909 | GPATCH8 | | 8016077 |
| NM_016558 | SCAND1 | | 8066031 |
| NM_024335 | IRX6 | | 7995674 |
| NM_004788 | UBE4A | 4120 | 7944195 |
| NM_021247 | PRM3 | | 7999431 |
| NR_024368 | FLJ45340 | | 7945344 |
| AK093358 | FAM27A | | 8161442 |
| NM_033467 | MMEL1 | | 7911767 |
| NR_002937 | C14orf19 | | 7973900 |
| NM_182973 | TMPRSS9 | | 8024467 |
| NM_018663 | PXMP2 | 4146 | 7967789 |
| NM_173728 | ARHGEF15 | | 8004842 |
| NM_004610 | TCP10 | | 8123374 or |
| | | | 8130787 |
| NM_006013 | RPL10 | 3010 | 8034416 or |
| | | | 8109821 |
| NM_015999 | ADIPOR1 | 4920 | 7923503 |
| NM_020209 | SHD | | 8024808 |
| NR_029829 | MIR194-2 | | 7949275 |
| NM_198998 | AQP12A | | 8060126 or |
| | | | 8049729 |
| NM_002233 | KCNA4 | | 7947270 |
| NM_022098 | XPNPEP3 | | 8073311 |
| NM_212550 | BLOC1S3 | | 8029640 |
| NM_198949 | NUDT1 | | 8131101 |
| NM_006626 | ZBTB6 | | 8163999 |
| NM_003795 | SNX3 | | 8128683 |
| NR_029663 | MIR15B | | 8083737 |
| NM_207397 | CD164L2 | | 7914084 |
| NM_014280 | DNAJC8 | | 7899422 |
| NM_001001915 | OR2G2 | 4740 | 7911207 |
| NM_004930 | CAPZB | | 7913169 |
| NM_015950 | MRPL2 | | 8126512 |
| NM_015509 | NECAP1 | | 7953715 |
| NM_006894 | FMO3 | 982 | 7907249 |
| NM_012483 | GNLY | | 8043236 |
| NM_001004333 | RNASEK | | 8004237, |
| | | | 8012000 or |
| | | | 8004241 |
| NM_147196 | TMIE | | 8079426 |
| NR_027850 | MTX2 | | 8046573 |
| NM_001037283 | EIF3B | 3013 | 8131111 |
| NM_178463 | C20orf166 | | 8063914 |
| BC090923 | C17orf90 | | 8019238 |
| NM_020825 | CRAMP1L | | 7992302 |
| NM_001080520 | DRGX | | 7933501 |
| NM_153232 | EID2 | | 8036749 |
| NM_012186 | FOXE3 | | 7901309 |
| NM_213605 | ZNF517 | | 8148932 |
| NM_178456 | C20orf85 | | 8063601 |
| NM_032547 | SCOC | | 8097521 |
| NM_001170738 | IQSEC3 | 4144 | 8067820 or |
| | | | 7952893 |
| NM_024591 | CHMP6 | 4144 | 8010550 |
| NM_014515 | CNOT2 | 3018 | 7957106 |
| NM_138387 | G6PC3 | | 8007561 |
| NM_024326 | FBXL15 | | 7930099 |
| NM_007262 | PARK7 | 5012 | 7897404 |
| NR_001527 | TTTY6 | | 8176782 or |
| | | | 8177347 |
| NM_001142588 | NFYC | 4612 | 7915345 |
| NM_001080489 | GLOD5 | | 8167356 |
| NM_199243 | GPR150 | | 8106976 |
| NM_000729 | CCK | | 8086391 |
| NM_024718 | C9orf86 | | 8159415 |
| NM_145729 | MRPL24 | | 7921121 |
| NM_030662 | MAP2K2 | 04010, 04012, | 8032761 |
| | | 04270, 04370, | |
| | | 04540, 04620, | |
| | | 04650, 04660, | |
| | | 04662, 04664, | |
| | | 04720, 04722, | |
| | | 04730, 04810, | |
| | | 04910, 04912, | |
| | | 04916, 05020, | |
| | | 05200, 05211, | |
| | | 05213, 05214, | |
| | | 05215, 05216, | |
| | | 05218, 05219, | |
| | | 05220, 05221, | |
| | | 05223 | |
| NM_058190 | FAM207A | | 8180370 or |
| | | | 8069174 |
| NM_001011 | RPS7 | 3010 | 8005877 or |
| | | | 8040036 |
| NM_014336 | AIPL1 | | 8011912 |
| NM_178354 | LCE1F | | 7905517 |
| NM_005470 | ABI1 | | 7932616 |
| NM_006357 | UBE2E3 | 04120, 04141 | 8166124 |
| NR_002182 | NACAP1 | | 8147693 |
| NM_001109763 | GSG1L | | 8000467 |
| NM_001085365 | MZT2A | | 8055287 |
| NM_001080468 | SYCN | | 8036699 |
| NM_003365 | UQCRC1 | 00190, 01100, | 8087100 |
| | | 04260, 05010, | |
| | | 05012, 05016 | |
| NM_153207 | AEBP2 | | 7954279 |
| NM_032477 | MRPL41 | | 8159687 |
| NM_152911 | PAOX | 4146 | 7931582 |
| NM_002488 | NDUFA2 | 00190, 01100, | 8114618 |
| | | 05010, 05012, | |
| | | 05016 | |
| NM_175895 | C12orf61 | | 7964642 |
| NM_004558 | NRTN | | 8024995 |
| NM_198850 | PHLDB3 | | 8037315 |
| NR_023343 | RNU4ATAC | | 8044961 |
| NM_004976 | KCNC1 | | 7938738 |
| NM_012184 | FOXD4L1 | | 8044634 |
| NM_054028 | SLC35G5 | | 8020162 |
| NM_006985 | NPIP | | 7999766 or |
| | | | 7993580 |
| NM_138334 | JOSD2 | | 8038571 |
| NM_014613 | FAF2 | | 8110169 |
| NM_022466 | IKZF5 | | 7936826 |
| NM_022731 | NUCKS1 | | 7909142 |
| NM_138284 | IL17D | | 7967969 |
| NM_004851 | NAPSA | 4142 | 8038556 |
| NM_031292 | PUS7L | | 7962427 |
| NM_017871 | CPSF3L | | 7911486 |
| NM_012476 | VAX2 | | 8042532 |
| NM_032343 | CHCHD6 | | 8082305 |
| NM_199287 | CCDC137 | | 8010629 |
| BC043417 | TMEM68 | | 8146480 |
| NR_029714 | MIR320A | | 8149705 |
| NM_207163 | LMOD2 | | 8135821 |
| NM_024339 | THOC6 | 3013 | 7992795 |
| NM_032411 | C2orf40 | | 8044143 |
| NM_016491 | MRPL37 | | 7901601 |
| NM_003089 | SNRNP70 | 3040 | 8030199 |
| NM_014360 | NKX2-8 | | 7978686 |
| NM_201589 | MAFA | 04930, 04950 | 8153409 |
| AK289373 | IGHG1 | | 8001104 |
| NM_005583 | LYL1 | | 8034608 |
| NM_022375 | OCLM | | 7908347 |
| NM_001006610 | SIAH1 | 04115, 04120, | 8001306 |
| | | 04310 | |
| NM_172229 | KREMEN2 | | 7992758 |
| NM_021996 | GBGT1 | 00603, 01100 | 8164833 |
| NM_194249 | DND1 | | 8114625 |
| NM_001142864 | PIEZO1 | | 7997827 |
| NM_178438 | LCE5A | | 7905483 |
| NM_001349 | DARS | 970 | 8055445 |
| NM_176806 | MOCS2 | 4122 | 8112020 |
| NR_024355 | BK250D10.8 | | 8073546 |
| NM_024768 | CCDC48 | | 8082465 |
| AK093505 | SPANXA2-OT1 | | 8175537 or |
| | | | 8170247 |
| NM_024754 | PTCD2 | | 8106107 |
| AK125905 | LOC100129581 | | 8050113 |
| NM_175619 | ZAR1 | | 8094968 |
| NM_001867 | COX7C | 00190, 01100, | 8106776 |
| | | 04260, 05010, | |
| | | 05012, 05016 | |
| NM_031492 | RBM4B | | 7949674 |
| NM_153376 | CCDC96 | | 8099242 |
| NM_002034 | FUT5 | 00601, 01100 | 8033064 |
| NM_181462 | MRPL55 | | 7924853 |
| NM_012257 | HBP1 | | 8135392 |
| NR_027780 | HMGXB4 | | 8072645 |
| NM_004822 | NTN1 | 4360 | 8004880 |
| NM_178454 | DRAM2 | | 7918474 |
| NM_015893 | PRLH | | 8049509 |
| NM_024313 | NOL12 | | 8072883 |
| NM_006522 | WNT6 | 04310, 04340, | 8048445 |
| | | 04916, 05200, | |
| | | 05217 | |
| NM_017832 | FAM206A | | 8157144 |
| NM_006947 | SRP72 | 3060 | 8095230 |
| NM_021948 | BCAN | | 7906205 |
| NM_001164405 | BHLHA9 | | 8003633 |
| NM_032830 | CIRH1A | 3008 | 7996891 |
| NM_080680 | COL11A2 | 04510, 04512, | 8125568 |
| | | 04974, 05146 | |
| NM_002622 | PFDN1 | | 8114567 |
| NM_002196 | INSM1 | | 8061303 |
| NM_002370 | MAGOH | 03013, 03015, | 7916274 |
| | | 03040 | |
| NR_024406 | LOC732275 | | 8003230 |
| NM_201653 | CHIA | 520 | 7903945 |
| AK296222 | LOC728093 | | 8112476 or |
| | | | 8105935 |
| NM_004343 | CALR | 04141, 04145, | 8026106 |
| | | 04612, 05142 | |
| NM_001003892 | DUPD1 | | 7934527 |
| NR_026837 | LOC283392 | | 7964976 |
| NM_006274 | CCL19 | 04060, 04062 | 8160879 |
| NM_014847 | UBAP2L | | 7905700 |
| NM_016396 | CTDSPL2 | | 7983335 |
| NM_015944 | AMDHD2 | 520 | 7992656 |
| NM_144567 | ANGEL2 | | 7924190 |
| NM_153270 | KLHL34 | | 8171786 |
| NM_080622 | ABHD16B | | 8064203 |
| NM_022753 | S100PBP | | 7899829 |
| AK290103 | LOC100287934 | | 7909990 or |
| | | | 7896754 |
| NR_000011 | SNORA70 | | 8170863 or |
| | | | 8025498 |
| NM_003968 | UBA3 | 4120 | 8088718 |
| NM_033257 | DGCR6L | | 8074565 |
| NM_016468 | COX16 | | 7979906 |
| NM_030642 | APOL5 | | 8072721 |
| NM_002565 | P2RY4 | 4080 | 8173366 |
| NM_014017 | LAMTOR2 | | 7906072 |
| NM_006192 | PAX1 | | 8061357 |
| NM_133450 | ANKS3 | | 7999177 |
| NM_024302 | MMP28 | | 8014282 |
| NM_016166 | PIAS1 | 04120, 04630, | 7984453 |
| | | 05160, 05200, | |
| | | 05222 | |
| NM_007037 | ADAMTS8 | | 7952752 |
| NR_033192 | CCDC59 | | 7965200 |
| NM_174895 | PCP2 | | 8033414 |
| NM_003279 | TNNC2 | 4020 | 8066590 |
| NM_001136262 | ATXN7L3B | | 7957242 |
| NM_004108 | FCN2 | | 8159211 |
| NM_032829 | C12orf34 | | 7958577 |
| NM_139136 | KCNC2 | | 7964987 |
| NM_033113 | ZNF628 | | 8031489 |
| NM_005687 | FARSB | 970 | 8059319 |
| NM_000180 | GUCY2D | 00230, 04740, | 8004763 |
| | | 04744 | |
| NM_024309 | TNIP2 | | 8099029 |
| NM_014554 | SENP1 | | 7962760 |
| NM_001013735 | FOXB2 | | 8155942 |
| NM_174922 | ADCK5 | | 8148850 |
| NM_032704 | TUBA1C | 04145, 04540, | 7955179 |
| | | 05130 | |
| NM_020385 | REXO4 | | 8164907 |
| NR_029894 | MIR148B | | 7955906 |
| NM_001040425 | U2AF1L4 | | 8036143 |
| NR_029945 | MIR423 | | 8006119 |
| U63828 | C20orf181 | | 8067754 |
| NM_133636 | HELQ | | 8101467 |
| NM_001102614 | SLC35G6 | | 8004428 |
| NM_004549 | NDUFC2 | 00190, 01100, | 7950644 |
| | | 05010, 05012, | |
| | | 05016 | |
| AY358101 | DBF4B | | 8007673 |
| NM_032842 | TMEM209 | | 8142912 |
| NM_001862 | COX5B | 00190, 01100, | 8043718 |
| | | 04260, 05010, | |
| | | 05012, 05016 | |
| NM_020535 | KIR2DL5A | 04612, 04650, | 8039884 |
| | | 05332 | |
| NM_003443 | ZBTB17 | 4110 | 7912675 |
| NM_080863 | ASB16 | | 8007584 |
| NM_030819 | GFOD2 | | 8002082 |
| NM_144576 | COQ10A | | 7956242 |
| NM_001006636 | GTDC1 | | 8055606 |
| NM_145178 | ATOH7 | | 7933978 |
| NR_029375 | WDR83 | | 8026013 |
| NM_005937 | MLLT6 | | 8014702 |
| NM_006040 | HS3ST4 | | 7994260 |
| NM_203451 | SERTM1 | | 7968650 |
| BC146842 | FAM26F | | 8121596 |
| NR_027693 | C1orf170 | | 7911371 |
| NM_152353 | CLDND2 | | 8038804 |
| NM_003806 | HRK | | 7966746 |
| NM_005169 | PHOX2A | | 7950136 |
| NM_006413 | RPP30 | 03008, 03013 | 7929116 |
| NM_032259 | WDR24 | | 7998280 |
| NM_005861 | STUB1 | 04120, 04141 | 7992021 |
| NM_178520 | TMEM105 | | 8019177 |
| NM_138433 | KLHDC7B | | 8074106 |
| NM_018221 | MOBKL1B | | 8053064 |
| BC043584 | C2orf27B | | 8055291 |
| NM_001166400 | MAGEA8 | | 8170412 |
| NM_004421 | DVL1 | 04310, 04330, | 7911506 |
| | | 04916, 05200, | |
| | | 05217 | |
| NM_001146340 | NKX1-2 | | 7936882 |
| NR_027386 | GUSBP3 | | 8112560, |
| | | | 8105991, |
| | | | 8177669, |
| | | | 8112469, |
| | | | 8112564 or |
| | | | 8177544 |
| NM_031948 | PRSS27 | | 7998843 |
| NM_017619 | RNPC3 | | 7903404 |
| NM_017730 | QRICH1 | | 8087271 |
| NM_001015072 | UFSP1 | | 8141643 |
| NM_005340 | HINT1 | | 8113873 |
| NM_033130 | SIGLEC10 | | 8038824 |
| NM_001098202 | HIC1 | | 8003719 |
| NM_001001502 | SNCB | | 8115948 |
| NM_178516 | EXOC3L1 | | 8001953 |
| NM_006757 | TNNT3 | | 7937749 |
| NR_029499 | MIR26A1 | | 8078663 |
| NR_027028 | GUSBP1 | | 8104621 |
| NM_012378 | OR8B8 | 4740 | 7952404 |
| AF462446 | LOC100288144 | | 7981488 |
| NM_173544 | FAM129C | | 8026787 |
| NM_017561 | FAM22F | | 8162479 |
| NM_006070 | TFG | 05200, 05216 | 8081316 |
| NM_030754 | SAA2 | | 7946983 |
| NM_001001419 | SMAD5 | 4350 | 8108238 |
| NM_207334 | FAM43B | | 7898653 |
| NM_020884 | MYH7B | 04530, 05416 | 8062064 |
| NM_053280 | ODF3 | | 7937263 |
| NM_001080475 | PLEKHM3 | | 8058509 |
| NM_021221 | LY6G5B | | 8178059 or |
| | | | 8118228 |
| NM_006571 | DCTN6 | 4962 | 8145660 |
| NM_006754 | SYPL1 | | 8142110 |
| NM_016451 | COPB1 | | 7946703 |
| NM_152758 | YTHDF3 | | 8146637 |
| NM_031274 | TEX13A | | 8174297 |
| NM_001029991 | METTL17 | | 7973116 |
| NM_182707 | PSG8 | | 8037236 |
| NM_021994 | ZNF277 | | 8135497 |
| NM_014042 | C11orf51 | | 7950128 |
| NM_145049 | UBLCP1 | | 8109597 |
| NM_002590 | PCDH8 | | 7971850 |
| NM_005182 | CA7 | 910 | 7996331 |
| NM_172089 | TNFSF12- | | 8004464 |
| | TNFSF13 | | |
| NM_003403 | YY1 | | 7976744 |
| NM_182560 | C14orf177 | | 7976635 |
| NM_012182 | FOXB1 | | 7984011 |
| NM_014302 | SEC61G | 03060, 04141, | 8139706 |
| | | 04145, 05110 | |
| NM_005262 | GFER | | 7992439 |
| NM_001001481 | UBE2W | 4120 | 8151401 |
| NM_001007561 | IRGQ | | 8037352 |
| NM_199290 | NACA2 | | 8017259 |
| NR_030174 | MIR494 | | 7976834 |
| NM_207379 | TMEM179 | | 7981490 |
| NR_003129 | RNF5P1 | | 8178188 or |
| | | | 8150356 |
| NM_015953 | NOSIP | | 8038395 |
| BC017770 | RBM8A | 03013, 03015, | 7904751 |
| | | 03040 | |
| NM_017964 | SLC30A6 | | 8041257 |
| NM_030761 | WNT4 | 04310, 04340, | 7913547 |
| | | 04916, 05200, | |
| | | 05217 | |
| NM_005406 | ROCK1 | 04062, 04270, | 8020382 |
| | | 04310, 04350, | |
| | | 04360, 04510, | |
| | | 04670, 04810, | |
| | | 05130, 05131 | |
| NM_017945 | SLC35A5 | | 8081667 |
| NM_173502 | PRSS36 | | 8001014 |
| NM_024766 | CAMKMT | | 8041745 |
| NM_181706 | DNAJC24 | | 7939093 |
| NM_033251 | RPL13 | 3010 | 8180283 or |
| | | | 8180284 |
| NM_178525 | ACTL9 | | 8033662 |
| NM_006351 | TIMM44 | | 8033465 |
| NM_181711 | GRASP | | 7955578 |
| NM_021223 | MYL7 | 04510, 04530, | 8139307 |
| | | 04670, 04810 | |
| NM_052936 | ATG4A | 4140 | 8169272 |
| NM_007108 | TCEB2 | 04120, 05200, | 7998852 |
| | | 05211 | |
| NM_080594 | RNPS1 | 03013, 03015 | 7998774 |
| NM_178167 | ZNF598 | | 7998679 |
| NM_001004453 | OR1L6 | 4740 | 8157688 |
| NR_000015 | SNORD55 | | 7901046 |
| NM_016093 | RPL26L1 | 3010 | 7945740 |
| NM_001694 | ATP6V0C | 00190, 01100, | 7992646 |
| | | 04142, 04145, | |
| | | 04966, 05110, | |
| | | 05120, 05323 | |
| NM_024112 | C9orf16 | | 8158177 |
| AB001736 | IGLJ3 | | 7981730 |
| NM_001136046 | ZMYND15 | | 8003926 |
| NM_024887 | DHDDS | 900 | 7899173 |
| NR_015370 | EPB41L4A-AS1 | | 8107321 |
| NM_000265 | NCF1 | 04062, 04145, | 8140227 or |
| | | 04380, 04666, | 8133518 |
| | | 04670, 05140 | |
| NM_002792 | PSMA7 | 3050 | 8067382 |
| NM_199186 | BPGM | 00010, 01100 | 8136341 |
| NM_024316 | LENG1 | | 8039139 |
| NM_021134 | MRPL23 | | 7937763 |
| NM_017610 | RNF111 | | 7983953 |
| NM_203434 | IER5L | | 8164554 |
| NM_080607 | VSTM2L | | 8062427 |
| NM_022573 | TSPY2 | | 8176419 |
| NR_029603 | MIR139 | | 7950195 |
| NM_080606 | BHLHE23 | | 8067585 |
| NM_194358 | RNF41 | 4144 | 7964021 |
| NM_016378 | VCX2 | | 8171240 |
| NM_015417 | SPEF1 | | 8064751 |
| NM_021231 | C19orf29 | | 8024682 |
| NM_020395 | INTS12 | | 8102162 |
| NM_018225 | SMU1 | | 8160622 |
| NM_173209 | TGIF1 | | 8180316, |
| | | | 8180319, |
| | | | 8180318 or |
| | | | 8180317 |
| NM_032319 | PRADC1 | | 8053004 |
| NM_145055 | C18orf25 | | 8021113 |
| NM_002818 | PSME2 | 03050, 04612 | 7978123 |
| NR_029891 | MIR326 | | 7950490 |
| NM_001303 | COX10 | 00190, 00860, | 8005089 |
| | | 01100 | |
| NM_152492 | CCDC27 | | 7897196 |
| NM_002235 | KCNA6 | | 7953264 |
| NM_153368 | GJD4 | | 7933073 |
| NM_016390 | C9orf114 | | 8164481 |
| NM_173829 | SREK1IP1 | | 8112337 |
| NM_172347 | KCNG4 | | 8003149 |
| NM_002620 | PF4V1 | 04060, 04062 | 8095694 |
| NM_015506 | MMACHC | 4977 | 7901102 |
| NM_000626 | CD79B | 4662 | 8017511 |
| NM_080650 | ATPBD4 | | 7987369 |
| NM_016145 | C19orf56 | | 8034448 |
| NM_016399 | TRIAP1 | | 7967056 |
| NM_005803 | FLOT1 | 4910 | 8124828, |
| | | | 8178419 or |
| | | | 8179688 |
| NM_002710 | PPP1CC | 04114, 04270, | 7966368 |
| | | 04510, 04720, | |
| | | 04810, 04910 | |
| NM_003090 | SNRPA1 | 3040 | 7991598 |
| NM_015530 | GORASP2 | | 8046306 |
| NM_052846 | EMILIN3 | | 8066294 |
| NM_053006 | TSSK2 | | 8071196 |
| NM_003134 | SRP14 | 3060 | 7987449 |
| NM_014765 | TOMM20 | | 7925174 |
| NM_007026 | DUSP14 | 4010 | 8006736 |
| NM_005125 | CCS | 5014 | 7941685 |
| NM_178161 | PTF1A | | 7926674 |
| BC016613 | C16orf11 | | 7991893 |
| NM_018471 | ZC3H15 | | 8057561 |
| NM_007265 | ECD | | 7934301 |
| NM_017521 | FEV | | 8059014 |
| NM_024041 | SCNM1 | | 7905355 |
| NM_020192 | C7orf36 | | 8132399 |
| NM_018403 | DCP1A | 3018 | 8088128 |
| NM_199350 | C9orf50 | | 8164562 |
| NM_182577 | ODF3L2 | | 8032051 |
| NM_001001657 | OR2Y1 | 4740 | 8116482 |
| BC126459 | C10orf95 | | 7936023 |
| NM_025108 | C16orf59 | | 7992614 |
| NM_133455 | EMID1 | | 8072189 |
| NR_003587 | MYO15B | | 8009873 |
| AK023602 | CEACAM21 | | 8037043 |
| NM_001025200 | CTRB2 | | 8002845 |
| NM_001037637 | BTF3 | | 8106181 |
| NM_213622 | STAMBP | 4144 | 8042772 |
| NM_004826 | ECEL1 | | 8059748 |
| NM_000198 | HSD3B2 | 00140, 01100 | 7904408 |
| NM_172341 | PSENEN | 04330, 05010 | 8027996 |
| NM_152344 | LSM12 | | 8015908 or |
| | | | 8150206 |
| NR_027392 | INTS4L2 | | 8133095 |
| NM_004914 | RAB36 | | 8074880 |
| AK124070 | LOC400707 | | 8029829 |
| NM_018233 | OGFOD1 | | 7995755 |
| NM_001078166 | SRSF1 | 3040 | 8016898 |
| NM_002434 | MPG | 3410 | 7991742 |
| NM_152704 | FAM123A | | 7970648 |
| NM_001038640 | GOLGA6A | | 7984985 |
| NM_178449 | PTH2 | | 8038362 |
| NM_021934 | C12orf44 | | 7955606 |
| NR_029614 | MIR182 | | 8142880 |
| NM_015161 | ARL6IP1 | | 7999834 |
| NM_001099692 | EIF5AL1 | | 7934753, |
| | | | 7928600 or |
| | | | 7928630 |
| NM_024409 | NPPC | | 8059739 |
| AY956766 | HSP90AB5P | | 8080987 |
| NM_032028 | TSSK1B | | 8113573 |
| NM_003574 | VAPA | 4530 | 8020129 |
| NM_001170330 | C4orf3 | | 8102518 |
| NM_007176 | C14orf1 | | 7980309 |
| NM_002155 | HSPA6 | 03040, 04010, | 7906764 |
| | | 04141, 04144, | |
| | | 04612, 05145 | |
| NM_005332 | HBZ | | 7991754 |
| NM_001080434 | LMTK3 | | 8038093 |
| NM_003352 | SUMO1 | 3013 | 8058335 |
| NM_022468 | MMP25 | | 7992811 |
| NM_000938 | POLR2B | 00230, 00240, | 8095269 |
| | | 01100, 03020, | |
| | | 05016 | |
| AK022260 | LOC401052 | | 8085272 |
| NM_205848 | SYT6 | | 7918716 |
| NM_022756 | MEAF6 | | 7914996 |
| NM_001137550 | LRRFIP1 | | 8049538 |
| NM_020943 | CWC22 | | 8057441 |
| NM_080596 | HIST1H2AH | 5322 | 8117543 |
| NR_033460 | PAGE3 | | 8173168 |
| NM_153812 | PHF13 | | 7897322 |
| NM_007243 | NRM | | 8178399, |
| | | | 8179683 or |
| | | | 8124806 |
| NM_003172 | SURF1 | | 8164896 |
| NM_001123355 | PPP6C | | 8164155 |
| NM_198285 | WDR86 | | 8143941 |
| NM_012403 | ANP32C | | 8103518 |
| NM_001906 | CTRB1 | 04972, 04974 | 7997264 |
| NM_080662 | PEX11G | 4146 | 8033385 |
| NM_024811 | CPSF7 | 3015 | 7948574 |
| NR_001548 | TTTY11 | | 8177085 |
| NM_002144 | HOXB1 | | 8016433 |
| BC119675 | FAM27E3 | | 8155569, |
| | | | 8161426 or |
| | | | 8161437 |
| AK095821 | LTB | 04060, 05323 | 8118147, |
| | | | 8124950, |
| | | | 8178512 or |
| | | | 8179768 |
| NM_018188 | ATAD3A | | 7896952 |
| NR_002226 | INGX | | 8173486 |
| NM_197960 | DPP8 | | 7989806 |
| NR_028409 | LINC00207 | | 8073698 |
| NM_201397 | GPX1 | 00480, 00590, | 8087405 |
| | | 05014, 05016 | |
| NM_052970 | HSPA12B | | 8060660 |
| NR_002992 | SNORA7B | | 8090565 |
| NM_005628 | SLC1A5 | 4974 | 8029854 |
| NM_003973 | RPL14 | 3010 | 8078984 |
| NR_029620 | MIR203 | | 7977214 |
| NM_182572 | ZSCAN1 | | 8031847 |
| NM_032110 | DMRTA2 | | 7915991 |
| AK126112 | FLJ44124 | | 8131042 |
| NM_175609 | ARFGAP1 | 4144 | 8064042 |
| NM_018466 | ALG13 | 00510, 01100 | 8169412 |
| NM_004379 | CREB1 | 04380, 04612, | 8047839 |
| | | 04916, 04962, | |
| | | 05016, 05215 | |
| NM_004801 | NRXN1 | 4514 | 8052091 |
| NM_001965 | EGR4 | | 8053022 |
| NM_022454 | SOX17 | 4310 | 8146462 |
| NM_198570 | VWC2 | | 8132805 |
| NR_002961 | SNORA22 | | 8133106 |
| NM_153029 | N4BP1 | | 8001317 |
| NM_012179 | FBXO7 | | 8072610 |
| NM_057093 | CRYBA2 | | 8059019 |
| NM_005507 | CFL1 | 04360, 04666, | 7949496 |
| | | 04810 | |
| NM_025215 | PUS1 | | 7959986 |
| NM_003868 | FGF16 | 04010, 04810, | 8168463 |
| | | 05200, 05218 | |
| NM_024798 | SNX22 | | 7984174 |
| NM_004095 | EIF4EBP1 | 03013, 04012, | 8145889 |
| | | 04150, 04910, | |
| | | 05221 | |
| NM_007254 | PNKP | | 8038458 |
| NM_012282 | KCNE1L | | 8174469 |
| NM_016239 | MYO15A | | 8005328 |
| NM_003322 | TULP1 | | 8125903 |
| NM_003456 | ZNF205 | | 7992845 |
| NM_020994 | CTAG2 | | 8176159 |
| NM_203400 | RPRML | | 8016320 |
| NM_000797 | DRD4 | 4080 | 7937433 |
| NM_001013620 | ALG10B | 00510, 01100 | 7954789 |
| NM_058219 | EXOSC6 | 3018 | 8002344 |
| NR_029687 | MIR152 | | 8016400 |
| BC011833 | C19orf52 | | 8025783 |
| AL137273 | DKFZP434I0714 | | 8097811 |
| NM_018120 | ARMC1 | | 8151066 |
| NR_028082 | COL18A1-AS1 | | 8070905 |
| NR_029693 | MIR125A | | 8030846 |
| NM_014233 | UBTF | | 8015969 |
| NM_199184 | C6orf108 | | 8126524 |
| NM_003015 | SFRP5 | 4310 | 7935528 |
| NM_014357 | LCE2B | | 7905505 |
| NM_019849 | SLC7A10 | | 8035993 |
| NM_005061 | RPL3L | 3010 | 7998643 |
| NM_015911 | ZNF691 | | 7900624 |
| NM_182706 | SCRIB | | 8153497 |
| NM_198458 | ZNF497 | | 8039759 |
| NM_015702 | MMADHC | | 8055672 |
| NM_004330 | BNIP2 | | 7989323 |
| NM_006913 | RNF5 | 4141 | 8118535 or |
| | | | 8179472 |
| NM_001136002 | TMEM229A | | 8142685 |
| NR_003496 | SNORD115-27 | | 7982060 |
| NM_016111 | TELO2 | | 7992271 |
| NM_003144 | SSR1 | 4141 | 8123767 |
| NM_001013658 | PTX4 | | 7998538 |
| NM_004675 | DIRAS3 | | 7916853 |
| NM_001127266 | TMEM129 | | 8098916 |
| NM_013403 | STRN4 | | 8037816 |
| NM_199135 | FOXD4L3 | | 8155661 |
| NM_022374 | ATL2 | | 8051589 |
| NM_024740 | ALG9 | 00510, 01100 | 7951633 |
| NM_016068 | FIS1 | | 8141717 |
| NM_013286 | RBM15B | | 8080093 |
| NM_015722 | CALY | | 7937200 |
| NM_001007540 | CDHR4 | | 8087481 |
| NM_020201 | NT5M | 00230, 00240, | 8005247 |
| | | 00760, 01100 | |
| NR_002326 | SNORA64 | | 7998666 |
| NM_152760 | SNX32 | | 7941408 |
| NM_001032288 | UBE2V1 | | 8180402 |
| NM_001077394 | DPH5 | | 7918034 |
| NM_033176 | NKX2-4 | | 8065327 |
| NM_178545 | TMEM52 | | 7911634 |
| NM_080476 | PIGU | 00563, 01100 | 8065762 |
| NM_014008 | CCDC22 | | 8167457 |
| NM_017510 | TMED9 | | 8110392 |
| NM_022077 | MANBAL | | 8062371 |
| NM_021938 | CELF5 | | 8024605 |
| NM_001774 | CD37 | 4640 | 8030277 |
| NM_001749 | CAPNS1 | | 8028172 |
| NM_001130064 | GAP43 | | 8081810 |
| NM_015667 | FAM75A7 | | 8161341, |
| | | | 8161471, |
| | | | 8155383 or |
| | | | 8155371 |
| NM_138493 | CCDC167 | | 8126095 |
| NM_004547 | NDUFB4 | 00190, 01100, | 8081945 or |
| | | 05010, 05012, | 8093314 |
| | | 05016 | |
| NM_015659 | RSL1D1 | | 7999520 |
| NM_014383 | ZBTB32 | | 8027947 |
| NM_001001888 | VCX3B | | 8165890 |
| NM_173825 | RABL3 | | 8089867 |
| NM_018690 | APOBR | | 7994353 |
| NM_002777 | PRTN3 | | 8024048 |
| NM_001001660 | LYRM5 | | 7954460 |
| NM_000967 | RPL3 | 3010 | 8076209 |
| NM_006917 | RXRG | 03320, 04920, | 7921955 |
| | | 05200, 05216, | |
| | | 05222, 05223 | |
| NM_182563 | C16orf79 | | 7998749 |
| NM_021197 | WFDC1 | | 7997582 |
| NM_001039165 | MRGPRE | | 7945859 |
| NR_015343 | LOC389458 | | 8131296 |
| NM_145057 | CDC42EP5 | | 8039273 |
| NM_016069 | PAM16 | | 7999112 |
| NM_203374 | ZNF784 | | 8039530 |
| NM_002952 | RPS2 | 3010 | 7998655 or |
| | | | 8013348 |
| NM_003217 | TMBIM6 | | 7955277 |
| NM_145665 | SPANXE | | 8175543 |
| NR_004845 | LOC644936 | | 8112883 |
| NM_007148 | RNF112 | | 8005586 |
| NM_000286 | PEX12 | 4146 | 8014264 |
| NM_152735 | ZBTB9 | | 8118721 |
| NM_198153 | TREML4 | | 8119427 |
| NM_005053 | RAD23A | 03420, 04141 | 8026122 |
| AK056484 | LOC441204 | | 8131965 |
| AB016901 | C6orf123 | | 8123405 |
| NR_004390 | SNORA57 | | 7940667 |
| NM_001144932 | PSMB5 | 3050 | 7977879 |
| NM_130760 | MADCAM1 | 04514, 04672 | 8023941 |
| NM_198469 | MORN5 | | 8157632 |
| NM_032120 | C7orf64 | | 8134211 |
| NM_013275 | ANKRD11 | | 7997907 or |
| | | | 8170322 |
| NM_145702 | TIGD1 | | 8059770 |
| NM_018082 | POLR3B | 00230, 00240, | 7958275 |
| | | 01100, 03020, | |
| | | 04623 | |
| NM_001001414 | NCCRP1 | | 8028600 |
| NM_198407 | GHSR | 4080 | 8092165 |
| NM_001028 | RPS25 | 3010 | 7939068 |
| NM_025224 | ZBTB46 | | 8067743 |
| NR_027393 | INTS4L1 | | 8133074 |
| NM_002412 | MGMT | | 7931379 |
| NM_018973 | DPM3 | 00510, 01100 | 7920633 |
| NM_176875 | CCKBR | 04020, 04080, | 7938090 |
| | | 04971 | |
| NM_144703 | LSM14B | | 8063835 |
| NM_080819 | GPR78 | | 8094093 |
| NM_175769 | TCF23 | | 8040827 |
| NM_139073 | SPATA3 | | 8049007 |
| NM_001007533 | DYSFIP1 | | 8019243 |
| NM_001313 | CRMP1 | | 8099172 |
| NM_012478 | WBP2 | | 8018482 |
| NM_014209 | ETV2 | | 8027920 |
| NM_017612 | ZCCHC8 | | 7967287 |
| NM_001516 | GTF2H3 | 03022, 03420 | 7959623 |
| NM_021138 | TRAF2 | 04010, 04141, | 8159476 |
| | | 04210, 04380, | |
| | | 04622, 04920, | |
| | | 05160, 05200, | |
| | | 05222 | |
| NM_021926 | ALX4 | | 7947563 |
| NM_001136156 | ZNF507 | | 8027439 |
| NM_207409 | C6orf126 | | 8118990 |
| NR_002962 | SNORA23 | | 7938329 |
| AK095315 | FBXO9 | | 8120269 or |
| | | | 8127128 |
| NM_181842 | ZBTB12 | | 8125201 or |
| | | | 8178683 |
| NM_015358 | MORC3 | | 8068460 |
| NM_024019 | NEUROG2 | | 8102368 |
| NM_001145408 | NONO | | 8168280 |
| NM_031954 | KCTD10 | | 7966202 |
| NM_207360 | ZC3H12D | | 8130073 |
| NM_004725 | BUB3 | 4110 | 7931187 |
| NM_001236 | CBR3 | 00590, 01100 | 8068413 |
| NM_015426 | POC1A | | 8087839 |
| NM_023018 | NADK | 00760, 01100 | 7911600 |
| NM_176801 | ADD1 | | 8093643 |
| NM_006367 | CAP1 | | 7900382 |
| NM_015846 | MBD1 | | 8023323 |
| NM_024815 | NUDT18 | | 8149646 |
| NM_012139 | SERGEF | | 7946933 |
| NM_007373 | SHOC2 | | 7930470 |
| NM_003508 | FZD9 | 04310, 04916, | 8133335 |
| | | 05200, 05217 | |
| NM_006090 | CEPT1 | 00564, 00565, | 7903908 |
| | | 01100 | |
| NM_199482 | PHOCN | | 8047228 |
| NM_178310 | SNAI3 | | 8003344 |
| NR_002914 | SNORD62A | | 8158862 or |
| | | | 8158864 |
| NM_014468 | VENTX | | 7931556 |
| AK094477 | LOC100129113 | | 8074307 |
| NM_003308 | TSPY1 | | 8176517, |
| | | | 8176484 or |
| | | | 8176544 |
| NM_015381 | FAM19A5 | | 8073929 |
| NR_002563 | SNORD27 | | 7948906 |
| NM_001416 | EIF4A1 | 3013 | 8004497 |
| NM_013318 | PRRC2B | | 8164649 |
| NM_001014283 | DCUN1D2 | | 7972912 |
| NM_207395 | ZNF324B | | 8031949 |
| NM_198446 | C1orf122 | | 7900192 |
| NM_001170741 | FAM22G | | 8156620 |
| NM_004219 | PTTG1 | 04110, 04114 | 8109639 |
| NM_018202 | TMEM57 | | 7899018 |
| NM_080723 | NRSN1 | | 8117178 |
| NM_018697 | LANCL2 | | 8132897 |
| NM_181837 | ORC3 | 4110 | 8121043 |
| NR_027020 | ANKRD30BL | | 8055297 |
| NM_153443 | KIR3DL3 | 4612 | 8031260 |
| NM_173470 | MMGT1 | | 8175360 |
| NM_025220 | ADAM33 | | 8064686 |
| NM_000399 | EGR2 | | 7933872 |
| NM_003857 | GALR2 | 4080 | 8010030 |
| AK097109 | LOC100131860 | | 7991047 |
| NM_152578 | FMR1NB | | 8170353 |
| NM_003690 | PRKRA | | 8057034 |
| NM_015113 | ZZEF1 | | 8003846 |
| NM_203422 | LRRN4CL | | 7948794 |
| NM_001099338 | FAM22A | | 7928653 or |
| | | | 7928645 |
| NM_030930 | UNC93B1 | | 8138147 or |
| | | | 8094144 |
| NM_001136263 | C2CD4C | | 8032034 |
| NM_003450 | ZNF174 | | 7992897 |
| AK056275 | FLJ31713 | | 8163533 |
| NM_001080493 | ZNF823 | | 8034315 |
| NM_022063 | FAM204A | | 7936578 |
| NM_177965 | C8orf37 | | 8151898 |
| NM_006236 | POU3F3 | | 8044109 |
| NM_173506 | LYPD4 | | 8037061 |
| NM_001004310 | FCRL6 | | 7906475 |
| NM_012192 | FXC1 | | 7938111 |
| NM_006136 | CAPZA2 | | 8135625 |
| NM_033258 | GNG8 | 4062 | 8037785 |
| NM_175876 | EXOC8 | | 7925043 |
| NM_138690 | GRIN3B | 4080 | 8024100 |
| NM_031890 | CECR6 | | 8074223 |
| NM_130468 | CHST14 | 532 | 7982745 |
| NM_006328 | RBM14 | | 7941694 |
| NM_001420 | ELAVL3 | | 8034263 |
| NM_014276 | RBPJL | 4330 | 8062948 |
| NM_183375 | PRSS48 | | 8097794 |
| NM_016512 | SPAG11B | | 8144460 |
| NM_012473 | TXN2 | | 8075778 |
| NM_020143 | PNO1 | | 8052719 |
| NM_018652 | GOLGA6B | | 7984961, |
| | | | 7984662 or |
| | | | 7985221 |
| NM_004911 | PDIA4 | 04141, 05110 | 8143684 |
| NM_007208 | MRPL3 | | 8090678 |
| NM_005547 | IVL | | 7905533 |
| NM_001009994 | RIPPLY2 | | 8120937 |
| NM_003505 | FZD1 | 04310, 04916, | 8134117 |
| | | 05200, 05217 | |
| NM_174889 | NDUFAF2 | | 8105499 |
| NM_002506 | NGF | 04010, 04210, | 7918869 |
| | | 04722 | |
| NM_022157 | RRAGC | | 7915160 |
| NR_004387 | SCARNA10 | | 7953383 |
| NM_001864 | COX7A1 | 00190, 04260, | 8036284 |
| | | 05010, 05012, | |
| | | 05016 | |
| NM_004314 | ART1 | | 7937882 |
| NM_001695 | ATP6V1C1 | 00190, 01100, | 8147724 |
| | | 04145, 04966, | |
| | | 05110, 05120, | |
| | | 05323 | |
| NM_023007 | JMJD4 | | 7924823 |
| NM_002377 | MAS1 | 04080, 04614 | 8123176 |
| NM_001142648 | SAR1A | 4141 | 7934122 |
| NM_020971 | SPTBN4 | | 8028806 |
| NM_004470 | FKBP2 | | 7940914 |
| NM_002032 | FTH1 | 860 | 8170360 |
| NM_001017928 | CCDC58 | | 8089988 |
| NM_001098169 | BSX | | 7952321 |
| NM_001145030 | C3orf77 | | 8079167 |
| NM_006294 | UQCRB | 00190, 01100, | 8151909 |
| | | 04260, 05010, | |
| | | 05012, 05016 | |
| NM_198492 | CLEC4G | | 8033433 |
| NM_138364 | PRMT10 | | 8103079 |
| NM_006362 | NXF1 | 03008, 03013, | 7948839 |
| | | 03015 | |
| NM_001130963 | TMEM194A | | 7964347 |
| NM_144720 | JAKMIP1 | | 8099200 |
| NM_031209 | QTRT1 | | 8025728 |
| NM_015092 | SMG1 | 3015 | 8000687 |
| NM_014637 | MTFR1 | | 8146649 |
| NM_003837 | FBP2 | 00010, 00030, | 8162492 |
| | | 00051, 01100, | |
| | | 04910 | |
| NM_001172129 | HCK | 04062, 04666 | 8061668 |
| NR_003260 | DNM1P46 | | 7986426 |
| NM_016013 | NDUFAF1 | | 7987642 |
| NM_004831 | MED26 | | 8035187 |
| NM_022728 | NEUROD6 | | 8138882 |
| NM_153442 | GPR26 | | 7931199 |
| NM_152864 | NKAIN4 | | 8067602 |
| NM_002309 | LIF | 04060, 04630 | 8072314 |
| NM_003951 | SLC25A14 | | 8169904 |
| NM_033341 | BIRC8 | | 8039078 |
| NM_018698 | NXT2 | 03008, 03013, | 8169352 |
| | | 03015 | |
| NM_198353 | KCTD8 | | 8100070 |
| NM_001127258 | HHIPL1 | | 7976669 |
| NR_027001 | LOC388152 | | 7991159 or |
| | | | 7991088 |
| NM_013270 | PRSS50 | | 8086660 |
| NM_001105572 | PLA2G2C | 00564, 00565, | 7913235 |
| | | 00590, 00591, | |
| | | 00592, 01100, | |
| | | 04010, 04270, | |
| | | 04370, 04664, | |
| | | 04730, 04912, | |
| | | 04972, 04975, | |
| | | 05145 | |
| NM_014846 | KIAA0196 | | 8152782 |
| NM_005205 | COX6A2 | 00190, 01100, | 8001041 |
| | | 04260, 05010, | |
| | | 05012, 05016 | |
| NM_130777 | XAGE2 | | 8167693 or |
| | | | 8172749 |
| NR_024151 | HSPA7 | | 7906775 |
| AY358690 | EEF1D | | 8153457 |
| NM_005800 | USPL1 | | 7968333 |
| NM_016486 | TMEM69 | | 7901135 |
| NM_020649 | CBX8 | | 8019010 |
| NM_152516 | COMMD1 | | 8042207 |
| NM_015933 | CCDC72 | | 7899346 |
| NM_016125 | RNFT1 | | 8017162 |
| NM_001485 | GBX2 | | 8059864 |
| NM_032730 | RTN4IP1 | | 8128606 |
| NM_001143938 | ZNF534 | | 8030939 |
| NM_017622 | C17orf59 | | 8012397 |
| AK313893 | CCDC82 | | 7951157 |
| NM_013334 | GMPPB | 00051, 00520, | 8087461 |
| | | 01100 | |
| NM_181788 | H1FNT | | 7955112 |
| NM_023039 | ANKRA2 | | 8112596 |
| NM_172239 | REXO1L1 | 3008 | 8151609 |
| NM_032834 | ALG10 | 00510, 01100 | 7954777 |
| NM_182546 | VSTM2A | | 8132851 |
| NM_015871 | ZNF593 | | 7899096 |
| NR_002912 | SNORA67 | | 8004508 |
| NM_002860 | ALDH18A1 | 00330, 01100 | 7935230 |
| NM_001166222 | CARNS1 | | 7941890 |
| NM_022662 | ANAPC1 | 04110, 04114, | 8054437 or |
| | | 04120, 04914 | 8043322 |
| NM_025263 | PRR3 | | 8117922 |
| NM_139062 | CSNK1D | 04340, 04540, | 8019463 |
| | | 04710 | |
| NM_080864 | RLN3 | | 8026265 |
| NR_002450 | SNORD68 | | 7997940 |
| NM_003224 | ARFRP1 | | 8067727 |
| NM_001145928 | SAP130 | | 8055104 |
| NM_006741 | PPP1R1A | 4720 | 7963826 |
| NM_139174 | ADAD2 | | 7997569 |
| NM_199193 | BRE | | 8041031 |
| NM_021190 | PTBP2 | | 7903188 |
| NM_005634 | SOX3 | | 8175528 |
| NM_021211 | ZBED5 | | 7946635 |
| NM_015267 | CUX2 | | 7958726 |
| NM_020755 | SERINC1 | | 8129317 |
| NM_144632 | TMEM182 | | 8044094 |
| NM_006036 | PREPL | | 8051928 |
| NM_015634 | KIAA1279 | | 7927955 |
| NM_004450 | ERH | | 7979864 |
| NM_015676 | C14orf109 | | 7976333 |
| NM_014655 | SLC25A44 | | 7906128 |
| NM_018641 | CHST12 | 00532, 00920 | 8131135 |
| NM_004793 | LONP1 | | 8033002 |
| NM_139209 | GRK7 | 04062, 04144, | 8083129 |
| | | 04744 | |
| NM_001641 | APEX1 | 3410 | 7973056 |
| NM_178510 | ANKK1 | | 7943943 |
| NM_182520 | C22orf15 | | 8071745 |
| NM_014675 | CROCC | | 7898377 |
| NM_001013632 | TCTEX1D4 | | 7915609 |
| NM_001080469 | FBXO46 | | 8037647 |
| NR_026833 | LOC400940 | | 8040077 |
| NM_006137 | CD7 | 4640 | 8019478 |
| NM_002987 | CCL17 | 04060, 04062 | 7996034 |
| NM_012112 | TPX2 | | 8061579 |
| NM_015910 | WDPCP | | 8052598 |
| NM_001003938 | HBM | | 7991758 |
| NR_029857 | MIR302B | | 8102406 |
| NM_003198 | TCEB3 | | 7898881 |
| NM_001690 | ATP6V1A | 00190, 01100, | 8081740 |
| | | 04145, 04966, | |
| | | 05110, 05120, | |
| | | 05323 | |
| NM_005153 | USP10 | | 7997633 |
| NR_029495 | MIR23A | | 8034698 |
| NM_001077621 | VPS37D | 4144 | 8133339 |
| NM_001100600 | MMD2 | | 8137931 |
| NR_027284 | LOC441177 | | 8123334 |
| NM_032819 | ZNF341 | | 8061946 |
| NM_017438 | SETD4 | | 8070215 |
| NM_005744 | ARIH1 | | 7984641 |
| NM_030571 | NDFIP1 | | 8108861 |
| NM_014275 | MGAT4B | 00510, 01100 | 8116316 |
| NR_002922 | SNORA13 | | 8107326 |
| NR_024532 | ALG2 | 00510, 01100 | 8162827 |
| NM_016172 | UBAC1 | | 8165064 |
| NM_001023560 | ZNF187 | | 8117667 |
| AK125652 | NIF3L1 | | 8047370 |
| NM_017638 | MED18 | | 7899448 |
| NM_032339 | C17orf37 | | 8014882 |
| NM_152494 | DCST1 | | 7905862 |
| NM_001001973 | ATP5C1 | 00190, 01100, | 7926084 |
| | | 05010, 05012, | |
| | | 05016 | |
| NM_016237 | ANAPC5 | 04110, 04114, | 7967149 |
| | | 04120, 04914 | |
| NM_030578 | B9D2 | | 8037018 |
| NM_021970 | LAMTOR3 | 4010 | 8101925 |
| NM_002568 | PABPC1 | 03013, 03015, | 8152079 |
| | | 03018 | |
| NM_001164416 | H2BFM | 5322 | 8169080 |
| NM_003353 | UCN | | 8051061 |
| NM_003477 | PDHX | 1100 | 7939329 |
| NM_181806 | AASDH | | 8100478 |
| NM_001081461 | JMJD6 | | 8018793 |
| NM_153358 | ZNF791 | | 8026007 |
| NM_006077 | MICU1 | | 7934255 |
| NM_001005470 | OR4B1 | 4740 | 7939865 |
| NM_022101 | CXorf56 | | 8180338 |
| NM_012318 | LETM1 | | 8098924 |
| NM_018241 | TMEM184C | | 8097704 |
| NR_026961 | LOC284837 | | 8070708 |
| NM_001008536 | TCHHL1 | | 7920135 |
| NM_203348 | MGC50722 | | 8180347 |
| NM_016091 | EIF3L | | 8072946 |
| NM_018129 | PNPO | 00750, 01100 | 8008064 |
| NM_138277 | C6orf25 | | 8178074 |
| NR_002950 | SNORA2A | | 7962827 |
| NR_033338 | C17orf70 | | 8019194 |
| L23320 | RFC1 | 03030, 03420, | 8165672 |
| | | 03430 | |
| NM_152888 | COL22A1 | | 8153101 |
| NR_026686 | PDIK1L | | 7899087 |
| NM_001004341 | ETV3L | | 7921222 |
| NM_014885 | ANAPC10 | 04110, 04114, | 8103005 |
| | | 04120, 04914 | |
| NM_025134 | CHD9 | | 8001402 |
| NM_001130059 | ATF7 | | 7963698 |
| NM_002484 | NUBP1 | | 7993185 |
| NM_001024594 | C1orf53 | | 7908525 |
| NM_005897 | IPP | | 7915775 |
| NM_032878 | ALKBH6 | | 8036242 |
| NM_000144 | FXN | | 8155699 |
| NM_032802 | SPPL2A | | 7988753 |
| NM_001051 | SSTR3 | 4080 | 8075906 |
| NM_016293 | BIN2 | | 7963289 |
| NM_147191 | MMP21 | | 7936928 |
| NM_013239 | PPP2R3B | 3015 | 8176986 or |
| | | | 8171087 |
| NM_001080483 | TMEM8C | | 8164931 |
| NM_002796 | PSMB4 | 3050 | 7905395 |
| NM_032663 | USP30 | | 7958439 |
| NM_172140 | IL29 | 04060, 04630 | 8028613 |
| NM_014018 | MRPS28 | | 8151471 |
| AK299337 | FAM65C | | 8066985 |
| NM_148961 | OTOS | | 8060094 |
| NM_001134875 | C14orf80 | | 7977418 |
| NM_001003703 | ATP5J | 00190, 01100, | 8069633 |
| | | 05010, 05012, | |
| | | 05016 | |
| NM_032048 | EMILIN2 | | 8019912 |
| NM_183401 | RNF14 | | 8108847 |
| NM_030943 | AMN | | 7977033 |
| NM_001827 | CKS2 | | 8156290 |
| NR_003049 | SNORD32B | | 8117746 |
| NM_053284 | WFIKKN1 | | 7991927 |
| NM_006830 | UQCR11 | 00190, 01100, | 8032284 |
| | | 04260, 05010, | |
| | | 05012, 05016 | |
| NM_016222 | DDX41 | | 8116096 |
| NM_001326 | CSTF3 | 3015 | 7947396 |
| NM_003434 | ZNF133 | | 8061154 |
| NM_018049 | PLEKHJ1 | | 8032455 |
| NR_027686 | LINC00176 | | 8064242 |
| NM_012183 | FOXD3 | | 7901913 |
| BC071695 | C11orf71 | | 7951781 |
| NR_024209 | RNF185 | | 8075477 |
| NM_000738 | CHRM1 | 04020, 04080, | 7948912 |
| | | 04810 | |
| NM_022465 | IKZF4 | | 7956105 |
| NM_004420 | DUSP8 | 4010 | 7945641 |
| NM_004550 | NDUFS2 | 00190, 01100, | 7906703 |
| | | 05010, 05012, | |
| | | 05016 | |
| NM_018691 | FAM114A2 | | 8115375 |
| NM_173680 | ZNF775 | | 8137228 |
| NM_138414 | CCDC101 | | 7994362 |
| NM_178554 | KY | | 8090872 |
| NM_178842 | CERS3 | | 7991546 |
| NM_152414 | BHLHE22 | | 8146645 |
| NM_005370 | RAB8A | 4972 | 8026520 |
| NR_001543 | TTTY14 | | 8177217 |
| NM_016585 | THEG | | 8032023 |
| NM_173575 | STK32C | | 7937089 |
| NM_004479 | FUT7 | 00514, 00601, | 8165398 |
| | | 01100 | |
| NM_078483 | SLC36A1 | 4974 | 8109350 |
| NM_016617 | UFM1 | | 7968670 |
| NM_017838 | NHP2 | 3008 | 8116168 |
| NM_000947 | PRIM2 | 00230, 00240, | 8120411 |
| | | 01100, 03030 | |
| BC063891 | C1orf201 | | 7913787 |
| NM_001010903 | C6orf222 | | 8125980 |
| NM_002751 | MAPK11 | 04010, 04370, | 8076978 |
| | | 04380, 04620, | |
| | | 04621, 04622, | |
| | | 04660, 04664, | |
| | | 04670, 04722, | |
| | | 04912, 04914, | |
| | | 05014, 05120, | |
| | | 05131, 05140, | |
| | | 05142, 05145, | |
| | | 05160 | |
| NM_001002255 | SUMO4 | 3013 | 8122684 |
| NM_198989 | DLEU7 | | 7971663 |
| BC063653 | LOC441239 | | 8139828 |
| NM_194328 | RNF38 | | 8161192 |
| NM_007097 | CLTB | 04142, 04144, | 8115918 |
| | | 05016, 05100 | |
| NM_152665 | TCTEX1D1 | | 7902158 |
| NM_020679 | MIF4GD | | 8018343 |
| NM_014736 | KIAA0101 | | 7989647 |
| NM_148886 | SMCR7 | | 8005435 |
| NM_139286 | CDC26 | 04110, 04114, | 8163481 |
| | | 04120, 04914 | |
| NM_138771 | CCDC126 | | 8131871 |
| NM_000252 | MTM1 | | 8170428 |
| NM_020862 | LRFN1 | | 8036707 |
| NM_173860 | HOXC12 | | 7955852 |
| NM_001169 | AQP8 | 4976 | 7994252 |
| NM_194248 | OTOF | | 8050942 |
| NM_019070 | DDX49 | | 8027100 |
| NR_027138 | C11orf36 | | 7937868 |
| NM_032539 | SLITRK2 | | 8170307 |
| NM_016310 | POLR3K | 00230, 00240, | 7998129 |
| | | 01100, 03020, | |
| | | 04623 | |
| NR_004388 | SCARNA14 | | 7989922 |
| NM_032556 | IL1F10 | | 8044563 |
| NM_016215 | EGFL7 | | 8159354 |
| NM_014402 | UQCRQ | 00190, 01100, | 8107998 |
| | | 04260, 05010, | |
| | | 05012, 05016 | |
| NM_016581 | ECSIT | 4010 | 8034286 |
| NM_005632 | SOLH | | 7991877 |
| NM_152779 | GLIPR1L1 | | 7957245 |
| NM_003807 | TNFSF14 | 4060 | 8033248 |
| NR_027241 | LOC388796 | | 8066247 |
| NM_031941 | USHBP1 | | 8035254 |
| NM_020967 | NCOA5 | | 8066668 |
| NM_153477 | UXT | | 8172358 |
| NM_025029 | MZT2B | | 8045142 |
| NM_001887 | CRYBB1 | | 8075118 |
| NR_027283 | LOC440461 | | 8009430 |
| NM_005171 | ATF1 | | 7955425 |
| NR_024075 | EMR4P | | 8033332 |
| NM_000409 | GUCA1A | 04740, 04744 | 8119515 |
| NM_001080461 | UNCX | | 8131087 |
| NM_001031834 | RAB40AL | | 8169006 |
| NM_014669 | NUP93 | 3013 | 7995843 |
| NM_014647 | KIAA0430 | | 7999642 |
| NM_001852 | COL9A2 | 4974 | 7915297 |
| NM_003544 | HIST1H4B | 5322 | 8124385 |
| NM_001037125 | UNKL | | 7998466 |
| NM_174937 | TCERG1L | | 7937059 |
| BC028365 | C7orf62 | | 8140852 |
| NM_198317 | KLHL17 | | 7896779 |
| NM_014044 | UNC50 | | 8043820 |
| NM_001436 | FBL | 3008 | 8036777 |
| NM_138568 | EXOC3L2 | | 8037513 |
| NM_018011 | ARGLU1 | | 7972723 |
| BC001912 | FAM195A | | 7991932 |
| NM_002688 | SEPTIN5 | 5012 | 8071259 |
| NM_001991 | EZH1 | | 8015685 |
| NM_080651 | MED30 | | 8148022 |
| NM_001099784 | FBXL19 | | 7994967 |
| NM_030755 | TMX1 | | 7974303 |
| NR_026925 | LOC151174 | | 8059985 |
| NR_031565 | MIR320C1 | | 8020419 |
| NM_030895 | ZNF696 | | 8148615 |
| NR_027238 | LOC654342 | | 8053722 |
| NR_026974 | C8orf77 | | 8148951 |
| NM_012384 | GMEB2 | | 8067709 |
| NM_138454 | NXNL1 | | 8035315 |
| NR_003004 | SCARNA22 | | 8093576 |
| NM_172251 | MRPL54 | | 8024708 |
| NM_001005188 | OR6X1 | 4740 | 7952373 |
| NM_013245 | VPS4A | 4144 | 7996919 |
| NM_012267 | HSPBP1 | 4141 | 8039440 |
| NM_002795 | PSMB3 | 3050 | 8006812 |
| NM_021066 | HIST1H2AJ | 5322 | 8124518 |
| NM_001344 | DAD1 | 00510, 01100, | 7977775 |
| | | 04141 | |
| NM_003348 | UBE2N | 4120 | 7965471 |

In one embodiment, a variant of a nucleotide sequence SEQ ID NO: X is a nucleotide sequence comprising at least 25 contiguous nucleotides, preferably of at least 50, 100, 150, 200 or at least 500 contiguous nucleotides of said nucleotide sequence SEQ ID NO: X.

In another embodiment, a variant of a nucleotide sequence SEQ ID NO: X is a nucleotide sequence comprising the nucleotide sequence SEQ ID NO: X and additional nucleic acids in 3′ and/or 5′ of SEQ ID NO: X, wherein the number of additional nucleic acids ranges from 1 to 500, preferably from 1 to 200, more preferably from 1 to 100 nucleotides.

In another embodiment, a variant of a nucleotide sequence SEQ ID NO: X is a nucleotide sequence that typically differs from said nucleotide sequence SEQ ID NO: X in one or more substitutions, deletions, additions and/or insertions. In one embodiment, said substitutions, deletions, additions and/or insertions may affect 1, 2, 3, 4, 5, 6, 7, 8, 9, or 10 nucleic acids.

In another embodiment, a variant of a nucleotide sequence SEQ ID NO: X is a nucleotide sequence of at least 25, preferably of at least 50, 100, 150, 200, 300, 400, 500, 1000, 1500, 2000 or 3000 nucleotides having at least 75%, 80%, 90%, 95%, or at least 96%, 97%, 98%, 99% identity with the nucleotide sequence SEQ ID NO: X.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. \2, 387 (1984); Genetics Computer Group, University of Wisconsin, Madison, Wis.), BLASTP, BLASTN, and FASTA (Altschul et al., J. MoI. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

In one embodiment of the invention, a fragment is a nucleotide sequence of at least 25 nucleotides, preferably of at least 50, 100, 150, 200 or at least 500 nucleotides. In one embodiment of the invention, a fragment of a sequence SEQ ID NO: X is a sequence of at least 25 contiguous nucleotides, preferably of at least 50, 100, 150, 200 or at least 500 contiguous nucleotides of SEQ ID NO: X.

In one embodiment, an equivalent of a nucleotide sequence SEQ ID NO: X, preferably of a gene having the sequence SEQ ID NO: X, is a nucleotide sequence, preferably a gene involved in the same pathway than the nucleotide sequence SEQ ID NO: X. A list of pathways and proteins involved in these pathways is available, for example, on the websites http://www.genome.jp/kegg/pathway.html or http://www.mybiosource.com/page.php ?name=pathways.

In another embodiment, cycling hypoxia markers are selected from the list of the 651 cycling hypoxia markers of Table 2 below, as well as their variants, fragments or equivalents. Table 2 comprises cycling hypoxia markers identified in the conditions of the Example and presenting a p-value after FDR correction lower than 0.01.

TABLE 2

| GeneBank | Name of the | | |
|---|---|---|---|
| Accession Number | marker | Pathways | Probeset |
| NR_002312 | RPPH1 | | 7977507 |
| BC018448 | MALAT1 | | 7949410 |
| NR_003287 | RN28S1 | | 7942875 or |
| | | | 8059576 |
| AF284753 | UIMC1 | | 7911343 or |
| | | | 8165703 |
| NM_014248 | RBX1 | 03420, 04110, | 8073334 |
| | | 04114, 04120, | |
| | | 04141, 04310, | |
| | | 04350, 04710, | |
| | | 05200, 05211 | |
| NM_177987 | TUBB8 | 04145, 04540, | 7911355 |
| | | 05130 | |
| NM_170601 | SIAE | | 7944867 |
| NM_001012708 | KRTAP5-3 | | 7945652 |
| NR_029710 | MIR193A | | 8006321 |
| NM_000981 | RPL19 | 3010 | 8006845 |
| NM_012217 | TPSD1 | | 7992191 |
| NM_003792 | EDF1 | | 8165309 |
| BC013044 | DNAJA2 | 4141 | 7995379 |
| NR_029824 | MIR128-2 | | 8078527 |
| NM_004352 | CBLN1 | | 8001329 |
| NM_001017 | RPS13 | 3010 | 7946812 |
| NM_001037160 | CYS1 | | 8050232 |
| NM_003731 | SSNA1 | | 8159609 |
| NM_006160 | NEUROD2 | | 8014865 |
| NM_001417 | EIF4B | 03013, 04150 | 7963575 |
| NM_017854 | TMEM160 | | 8037853 |
| NM_016057 | COPZ1 | | 7955896 |
| NM_152568 | NKX6-3 | | 8150433 |
| NM_016170 | TLX2 | | 8042896 |
| NR_002715 | RN7SL1 | | 8040338 |
| NM_016564 | CEND1 | | 7945536 |
| AK302042 | LOC440518 | | 8027343 |
| NM_014206 | C11orf10 | | 7948606 |
| NR_033335 | SNORA70G | | 7964830 |
| NM_003094 | SNRPE | 3040 | 8160033 or |
| | | | 7908988 |
| NM_012322 | LSM5 | 03018, 03040 | 8138912 |
| NM_145232 | CTU1 | 4122 | 8038782 |
| NR_029583 | MIR197 | | 7903717 |
| NM_032231 | FAM96A | | 7989611 |
| NR_024583 | POM121L8P | | 8071168 |
| NM_007241 | SNF8 | 4144 | 8016508 |
| NM_000307 | POU3F4 | | 8168567 |
| NM_013299 | SAC3D1 | | 7941122 |
| NM_005608 | PTPRCAP | | 7949792 |
| NM_006327 | TIMM23 | | 7927548 |
| NM_016424 | LUC7L3 | | 8016733 |
| NM_144615 | TMIGD2 | | 8032782 |
| NM_001135086 | PRSS41 | | 7992716 |
| NM_003512 | HIST1H2AC | 5322 | 8117372 |
| NM_000863 | HTR1B | 4080 | 8127692 |
| NM_145203 | CSNK1A1L | 04310, 04340 | 7971071 |
| NR_000009 | SNORD4B | | 8005957 |
| NM_001080113 | C14orf184 | | 7980859 |
| AK123383 | LOC642648 | | 8076747 |
| NM_032479 | MRPL36 | | 8110861 |
| NM_031210 | SLIRP | | 7975989 |
| NM_023002 | HAPLN4 | | 8035646 |
| NM_182532 | TMEM61 | | 7901687 |
| NM_003538 | HIST1H4A | 5322 | 8117334 |
| AK125166 | LOC441268 | | 8141166 |
| NM_001001521 | UGP2 | 00040, 00052, | 8052624 |
| | | 00500, 00520, | |
| | | 01100 | |
| NR_001445 | RN7SK | | 8120249 |
| NM_001551 | IGBP1 | | 8168087 |
| NM_138417 | KTI12 | | 7916130 |
| NM_031213 | FAM108A1 | | 8032371, |
| | | | 7904869, |
| | | | 7904948, |
| | | | 7924230 or |
| | | | 8074842 |
| BC001181 | FAM173A | | 7992043 |
| NM_001031 | RPS28 | 3010 | 8005471, |
| | | | 8025395 or |
| | | | 7942824 |
| NM_004175 | SNRPD3 | 03040, 05322 | 8071920 |
| NM_001044370 | MPPED1 | | 8073623 |
| BC005079 | C2orf42 | | 8052834 |
| NM_003542 | HIST1H4C | 5322 | 8117368 |
| BC033986 | LOC440934 | | 8048712 |
| NM_001082575 | RBFOX3 | | 8018993 |
| NM_017900 | AURKAIP1 | | 7911532 or |
| | | | 8039923 |
| NM_001024598 | HES3 | | 7897280 |
| NM_022061 | MRPL17 | | 7946267 |
| NM_001029 | RPS26 | 3010 | 8007797 |
| NM_016060 | MED31 | | 8011968 |
| NM_012394 | PFDN2 | | 7921786 |
| NM_015965 | NDUFA13 | 00190, 05010, | 8027205 |
| | | 05012, 05016 | |
| NM_080603 | ZSWIM1 | | 8063074 |
| NM_021104 | RPL41 | 3010 | 7957530, |
| | | | 7965467, |
| | | | 7982129, |
| | | | 8105432 or |
| | | | 8075691 |
| NM_000847 | GSTA3 | 00480, 00980, | 8127087 |
| | | 00982 | |
| NM_032753 | RAX2 | | 8032601 |
| NM_003684 | MKNK1 | 04010, 04910 | 7915846 |
| NM_003577 | UTF1 | | 7931553 |
| NM_022363 | LHX5 | | 7966631 |
| NM_001037495 | DYNLL1 | 4962 | 7967067 |
| NM_004609 | TCF15 | | 8064370 |
| AK098732 | TRAP1 | | 7992954 |
| NM_144999 | LRRC45 | | 8010719 |
| NM_001018138 | NME2 | 00230, 00240, | 8180388, |
| | | 01100 | 8180389, |
| | | | 8180387 or |
| | | | 8180386 |
| NM_002528 | NTHL1 | 3410 | 7998692 |
| NM_006087 | TUBB4 | 04145, 04540, | 8025051 |
| | | 05130 | |
| NM_003493 | HIST3H3 | 5322 | 7924884 |
| NR_026800 | KIAA0125 | | 7977440 |
| NM_015456 | COBRA1 | | 8159654 |
| NM_006088 | TUBB2C | 04145, 04540, | 8165496 |
| | | 05130 | |
| NM_002307 | LGALS7 | | 8036584 or |
| | | | 8028546 |
| NM_181887 | UBE2D3 | 04120, 04141 | 8180330, |
| | | | 8180335, |
| | | | 8180334, |
| | | | 8180331, |
| | | | 8180333, |
| | | | 8180329 or |
| | | | 8180332 |
| NM_001348 | DAPK3 | 05200, 05219 | 8032718 |
| NM_005319 | HIST1H1C | | 8124397 |
| NM_178536 | LCN12 | | 8159501 |
| NR_003666 | SPDYE7P | | 8133209 |
| AK125308 | LOC100129484 | | 8137962 |
| NM_020412 | CHMP1B | 4144 | 8020179 |
| NM_003550 | MAD1L1 | 04110, 04914 | 8137805 |
| NM_032527 | ZGPAT | | 8064156 |
| NR_003051 | RMRP | | 8161024 |
| NR_029681 | MIR140 | | 7997008 |
| NM_006858 | TMED1 | | 8034101 |
| NM_006312 | NCOR2 | 4330 | 7959772 |
| AK095987 | FLJ38668 | | 8054449 |
| ENST00000427835 | C20orf61 | | 8065013 |
| NM_001144936 | C11orf95 | | 7949015 |
| NM_173547 | TRIM65 | | 8018502 |
| NM_014370 | SRPK3 | | 8170753 |
| NM_005574 | LMO2 | | 7947450 |
| NM_001007595 | C2CD4B | | 7989473 |
| NM_001168 | BIRC5 | 05200, 05210 | 8018860 |
| NM_021012 | KCNJ12 | | 8005726 |
| NM_144589 | COMTD1 | | 7934544 |
| NM_016589 | TIMMDC1 | | 8081867 |
| NM_012315 | KLK9 | | 8038716 |
| NM_006292 | TSG101 | 4144 | 7947015 |
| NM_033055 | HIAT1 | | 7903294 |
| NM_001113201 | NACA | | 7964262 |
| NM_181838 | UBE2D2 | 04120, 04141, | 8108435 |
| | | 05131 | |
| NM_005973 | PRCC | | 7906235 |
| NM_005274 | GNG5 | 4062 | 8174509 |
| NM_006770 | MARCO | 4145 | 8044773 |
| NM_014674 | EDEM1 | 4141 | 8085116 |
| NM_145657 | GSX1 | | 7968260 |
| NM_002003 | FCN1 | | 8165011 |
| NM_003001 | SDHC | 00020, 00190, | 8011212 |
| | | 01100, 05010, | |
| | | 05012, 05016 | |
| NM_018942 | HMX1 | | 8104136 |
| NM_006848 | CCDC85B | | 7941457 |
| NM_032338 | LLPH | | 7956876 |
| NM_015971 | MRPS7 | | 8009784 |
| NM_020180 | CELF4 | | 8022952 |
| NM_001080495 | TNRC18 | | 8137959 |
| NM_006181 | NTN3 | 4360 | 7992632 |
| AK094921 | LOC100131763 | | 8049950 |
| NM_198545 | C1orf187 | | 7897737 |
| NM_002066 | GML | | 8148565 |
| NM_031899 | GORASP1 | | 8086317 |
| NM_012452 | TNFRSF13B | 04060, 04672, | 8013061 |
| | | 05340 | |
| NM_138574 | HDGFL1 | | 8117172 |
| NM_024816 | RABEP2 | | 8000616 |
| NM_022097 | CHP2 | 04010, 04020, | 7994123 |
| | | 04114, 04210, | |
| | | 04310, 04360, | |
| | | 04370, 04650, | |
| | | 04660, 04662, | |
| | | 04720, 05010, | |
| | | 05014 | |
| NM_006801 | KDELR1 | 5110 | 8038078 |
| NM_004939 | DDX1 | | 8040386 |
| NM_130784 | SYCE1 | | 7937247 |
| NM_019082 | DDX56 | | 8139392 |
| NM_001039916 | ZNF384 | | 7953390 |
| NM_016602 | CCR10 | 04060, 04062, | 8015681 |
| | | 04672 | |
| NR_024591 | POM121L1P | | 8074714 |
| NM_020064 | BARHL1 | | 8158912 |
| NM_006356 | ATP5H | 00190, 01100, | 8018288 |
| | | 05010, 05012, | |
| | | 05016 | |
| NM_012249 | RHOQ | 4910 | 8041808 |
| NR_002951 | SNORA2B | | 7962829 |
| NR_004430 | RNU1-1 | | 7919269, |
| | | | 7919349, |
| | | | 7898375, |
| | | | 7898411, |
| | | | 7912800, |
| | | | 7912850, |
| | | | 7919576, |
| | | | 7973896 or |
| | | | 7978568 |
| NM_001126128 | PROK2 | | 8088813 |
| NM_016063 | HDDC2 | | 8129363 |
| NM_005706 | TSSC4 | | 7937813 |
| NR_002781 | TSPY26P | | 8065603 |
| NM_175064 | SPDYE1 | | 8140424 or |
| | | | 8140454 |
| NM_138350 | THAP3 | | 7897329 |
| AY730278 | CENPVL1 | | 8167652 or |
| | | | 8172715 |
| NM_002669 | PLRG1 | 3040 | 8103289 |
| NM_006476 | ATP5L | 00190, 01100 | 7944216 |
| NM_031909 | C1QTNF4 | | 7947928 |
| NM_032805 | ZSCAN10 | | 7998921 |
| NM_001804 | CDX1 | | 8109226 |
| NM_014976 | PDCD11 | | 7936096 |
| AK097604 | LOC100130285 | | 7998265 |
| NM_003168 | SUPT4H1 | | 8016982 |
| NM_016835 | MAPT | 04010, 05010 | 8016263 |
| NM_001002 | RPLP0 | 3010 | 8109750 |
| NM_016305 | SS18L2 | | 8079074 |
| NM_001033113 | ENTPD8 | 00230, 00240 | 8165538 |
| NM_003926 | MBD3 | | 8032275 |
| NM_031280 | MRPS15 | | 7914940 |
| NR_026676 | RPS2P32 | | 8131869 |
| NM_145803 | TRAF6 | 04010, 04120, | 7947540 |
| | | 04144, 04380, | |
| | | 04620, 04621, | |
| | | 04622, 04722, | |
| | | 05140, 05142, | |
| | | 05145, 05160, | |
| | | 05200, 05222 | |
| NM_000858 | GUK1 | 00230, 01100 | 7910241 |
| NM_139172 | TMEM190 | | 8031475 |
| NM_018047 | RBM22 | 3040 | 8115168 |
| NM_182702 | PRSS42 | | 8086683 |
| NM_003859 | DPM1 | 00510, 01100 | 8067017 |
| NM_003002 | SDHD | 00020, 00190, | 7943853 or |
| | | 01100, 05010, | 7899016 |
| | | 05012, 05016 | |
| NR_026716 | KIR3DX1 | | 8031200 |
| NM_015719 | COL5A3 | 04510, 04512, | 8033825 |
| | | 04974, 05146 | |
| L20860 | SEPT5-GP1BB | | 8071272 |
| NM_101395 | DYRK1A | | 8068551 |
| NM_014419 | DKKL1 | | 8030292 |
| NR_026557 | PLK5 | | 8024331 |
| BC043386 | C19orf68 | | 8029996 |
| NM_001080440 | OTOL1 | | 8083770 |
| NM_144578 | MAPK1IP1L | | 7974455 |
| NM_012145 | DTYMK | 00240, 01100 | 8060286 or |
| | | | 8077262 |
| NM_002804 | PSMC3 | 3050 | 7947867 |
| NM_001172743 | RAO | | 8171539 |
| NM_016547 | SDF4 | | 7911422 |
| NM_175741 | C15orf55 | | 7982516 |
| NM_003910 | BUD31 | 3040 | 8134589 |
| NM_014342 | MTCH2 | | 7947934 |
| NM_001013 | RPS9 | 3010 | 8180398 or |
| | | | 8031152 |
| NM_021646 | ZNF500 | | 7999196 |
| AY341951 | FAM138D | | 7960172 |
| NM_005034 | POLR2K | 00230, 00240, | 8147654 |
| | | 01100, 03020, | |
| | | 05016 | |
| NM_001005922 | KRTAP5-1 | | 7945645 |
| NM_001105669 | TTC24 | | 7906177 |
| NM_006043 | HS3ST2 | 534 | 7994052 |
| NM_173641 | EPHA10 | | 7915078 |
| NM_001010908 | C1QL3 | | 7932308 |
| NM_001164094 | COPS7A | | 7953395 |
| NM_014582 | OBP2A | | 8180231 |
| NM_024319 | C1orf35 | | 7924842 |
| NM_003375 | VDAC2 | 04020, 05012, | 8042335 |
| | | 05016 | |
| NM_007374 | SIX6 | | 7974793 |
| NM_001003684 | UQCR10 | 00190, 01100, | 8072274 |
| | | 04260, 05010, | |
| | | 05012, 05016 | |
| NR_000025 | SNORD15B | | 7942594 |
| AF304442 | C21orf118 | | 8068046 |
| NM_016312 | WBP11 | 3040 | 7961489 |
| NM_080875 | MIB2 | | 7896985 |
| NM_005922 | MAP3K4 | 04010, 04912 | 8130624 |
| NR_002576 | SNORA21 | | 8014755 |
| NM_018462 | BRK1 | 4810 | 8085287 |
| NR_003013 | SCARNA16 | | 8010137 |
| NM_005609 | PYGM | 00500, 04910 | 7949124 |
| NM_016438 | HIGD1B | | 8007701 |
| NM_004855 | PIGB | 00563, 01100 | 7983811 |
| NR_026713 | FAM182A | | 8065527 |
| NM_003537 | HIST1H3B | 5322 | 8124388 |
| NM_001040436 | YARS2 | 970 | 7962203 |
| NM_014171 | CRIPT | | 8041813 |
| NM_053056 | CCND1 | 04110, 04115, | 7950012 |
| | | 04310, 04510, | |
| | | 04630, 05200, | |
| | | 05210, 05212, | |
| | | 05213, 05214, | |
| | | 05215, 05216, | |
| | | 05218, 05219, | |
| | | 05220, 05221, | |
| | | 05222, 05223, | |
| | | 05416 | |
| AF067420 | IGHA1 | | 7995263 |
| NM_198180 | QRFP | | 8164630 |
| BC004224 | SHANK2-AS3 | | 7942228 |
| NM_019107 | C19orf10 | | 8032863 |
| NM_001800 | CDKN2D | 4110 | 8034075 |
| NM_014860 | SUPT7L | | 8051204 |
| NM_000183 | HADHB | 00062, 00071, | 8040639 |
| | | 00280, 01100 | |
| NM_001258 | CDK3 | | 8010021 |
| NM_005247 | FGF3 | 04010, 04810, | 7950036 |
| | | 05200, 05218 | |
| NM_007255 | B4GALT7 | 00532, 00534, | 8110399 |
| | | 01100 | |
| NM_016199 | LSM7 | 03018, 03040 | 8032480 |
| BC004943 | MGC10814 | | 8035551 |
| NR_024593 | POM121L10P | | 8075024 |
| NM_145719 | TIGD3 | | 7941255 |
| NM_001164440 | ANKRD33B | | 8104499 |
| NM_174923 | CCDC107 | | 8155073 |
| NM_015276 | USP22 | | 8013486 |
| NM_001164447 | FAM90A10 | | 8144448 |
| NM_133261 | GIPC3 | | 8024676 |
| NM_001037984 | SLC38A10 | | 8019149 |
| NM_006855 | KDELR3 | 5110 | 8073015 |
| NM_021944 | C14orf93 | | 7977868 |
| NM_005430 | WNT1 | 04310, 04340, | 7955170 |
| | | 04916, 05200, | |
| | | 05217 | |
| NM_199044 | NSUN4 | | 7901219 |
| AK125575 | ZNF425 | | 8143708 |
| NM_032344 | NUDT22 | | 7940884 |
| NM_018090 | NECAP2 | | 7898337 |
| NM_000377 | WAS | 04062, 04520, | 8167334 |
| | | 04666, 04810, | |
| | | 05100, 05130, | |
| | | 05131 | |
| NM_001001410 | C16orf42 | | 7998449 |
| NM_148172 | PEMT | 00564, 01100 | 8013120 |
| NM_001032363 | C1orf151 | | 7898574 |
| NM_002949 | MRPL12 | | 8010664 |
| NM_012094 | PRDX5 | 4146 | 7940996 |
| NR_001555 | GOLGA2P2Y | | 8176910 or |
| | | | 8177413 |
| NM_025072 | PTGES2 | 00590, 01100 | 8164362 |
| NM_138983 | OLIG1 | | 8068235 |
| NM_016568 | RXFP3 | | 8104781 |
| NM_152914 | C17orf103 | | 8013509 |
| NM_014170 | GTPBP8 | | 8081676 |
| NM_003513 | HIST1H2AB | 5322 | 8124391 |
| NM_003278 | CLEC3B | | 8079305 |
| NM_004637 | RAB7A | 04144, 04145, | 8082431 |
| | | 05146 | |
| NM_001144954 | C5orf47 | | 8110068 |
| NM_001048183 | PHACTR4 | | 7899455 |
| NM_004551 | NDUFS3 | 00190, 01100, | 7939825 |
| | | 05010, 05012, | |
| | | 05016 | |
| NM_006808 | SEC61B | 03060, 04141, | 8156838 |
| | | 04145, 05110 | |
| NM_020199 | C5orf15 | | 8114138 |
| NM_030811 | MRPS26 | | 8060599 |
| NR_026581 | MLF2 | | 7960689 |
| NM_002297 | LCN1 | | 8159255 |
| BC104424 | FAHD2B | | 8043682 |
| NM_005714 | KCNK7 | | 7949444 |
| NM_001099435 | SPDYE5 | | 8133654 |
| BC035374 | TCEANC2 | | 7901592 |
| NM_005091 | PGLYRP1 | | 8037742 |
| NM_001013653 | LRRC26 | | 8165453 |
| NM_182498 | ZNF428 | | 8037355 |
| NM_001024678 | LRRC24 | | 8153868 |
| NM_001114600 | C1orf144 | | 7898328 |
| NM_001614 | ACTG1 | 04145, 04510, | 8019183 |
| | | 04520, 04530, | |
| | | 04670, 04810, | |
| | | 05100, 05110, | |
| | | 05130, 05131, | |
| | | 05410, 05412, | |
| | | 05414, 05416 | |
| NM_000383 | AIRE | 04120, 05340 | 8069037 |
| NR_002911 | SNORA71A | | 8066258 |
| NM_001135580 | C19orf71 | | 8024655 |
| NM_145272 | C17orf50 | | 8006569 |
| NM_000154 | GALK1 | 00052, 00520, | 8018428 |
| | | 01100 | |
| AY358234 | LOC100129831 | | 8024444 |
| NM_001100418 | C19orf60 | | 8027032 |
| NM_020996 | FGF6 | 04010, 04810, | 7960407 |
| | | 05200, 05218 | |
| NM_001297 | CNGB1 | 04740 04744 | 8001615 |
| AK128227 | LOC100130236 | | 7919299 or |
| | | | 7919380 |
| NM_002494 | NDUFC1 | 00190, 01100, | 8102839 |
| | | 05010, 05012, | |
| | | 05016 | |
| NM_198175 | NME1 | 00230, 00240, | 8008517 |
| | | 01100 | |
| NM_000479 | AMH | 04060, 04350 | 8024429 |
| NM_001145250 | SP9 | | 8056825 |
| NR_002144 | LOC407835 | | 8136065 |
| NM_014110 | PPP1R8 | | 7914139 |
| NM_152898 | FERD3L | | 8138450 |
| NR_030170 | MIR202 | | 7937148 |
| NM_177398 | LMX1A | | 7921936 |
| NM_178348 | LCE1A | | 7905528 |
| NM_005444 | RQCD1 | 3018 | 8048340 |
| NM_173806 | PDZD9 | | 8000229 |
| NM_006857 | SNRNP27 | 3040 | 8042495 |
| NM_033644 | FBXW11 | 04114, 04120, | 8115765 |
| | | 04310, 04340, | |
| | | 04710, 05131 | |
| NM_001164456 | FAM90A13 | | 8149204, |
| | | | 8144428, |
| | | | 8144432, |
| | | | 8144436 or |
| | | | 8144444 |
| NM_020070 | IGLL1 | 5340 | 8074909 |
| NM_005007 | NFKBIL1 | | 8118127, |
| | | | 8177967 or |
| | | | 8179249 |
| NM_177542 | SNRPD2 | 3040 | 8037642 |
| NM_020637 | FGF22 | 04010, 04810, | 8023990 |
| | | 05200, 05218 | |
| NM_144727 | CRYGN | | 8143949 |
| NM_015568 | PPP1R16B | | 8062557 |
| NM_018250 | INTS9 | | 8150014 |
| NM_003517 | HIST2H2AC | 5322 | 7905088 |
| NM_032998 | DEDD | | 7921793 |
| NM_173660 | DOK7 | | 8093807 |
| NM_015679 | TRUB2 | | 8164428 |
| NM_030657 | LIM2 | | 8038815 |
| NM_015918 | POP5 | 03008, 03013 | 7967084 |
| NR_029833 | MIR30C1 | | 7900490 |
| NM_001004 | RPLP2 | 3010 | 7937476 |
| NM_152778 | MFSD8 | 4142 | 8102730 |
| NM_032595 | PPP1R9B | | 8016628 |
| NM_006686 | ACTL7B | | 8163019 |
| AK291454 | UBE2K | 4120 | 8099918 |
| NM_000983 | RPL22 | 3010 | 7911989 |
| NM_053049 | UCN3 | | 7925950 |
| NM_001030047 | KLK3 | 05200, 05215 | 8030753 |
| NR_003502 | ZNRF2P1 | | 8132209 |
| NM_016734 | PAX5 | | 8161211 |
| NM_052945 | TNFRSF13C | 04060, 04672, | 8076387 |
| | | 05340 | |
| NM_006299 | ZNF193 | | 8117655 |
| NM_002764 | PRPS1 | 00030, 00230, | 8169240 |
| | | 01100 | |
| NM_001024675 | C20orf134 | | 8061944 |
| NM_014064 | METTL11A | | 8158544 |
| NM_001002252 | ARL6IP4 | | 7959549 |
| NM_004640 | DDX39B | 03013, 03015, | 8178476, |
| | | 03040 | 8179750 or |
| | | | 8124926 |
| NM_173514 | SLC38A9 | | 8112121 |
| NM_018955 | UBB | 5012 | 8005166 |
| NM_001130861 | CLDN5 | 04514, 04530, | 8074473 |
| | | 04670, 05160 | |
| NM_006432 | NPC2 | 4142 | 7980146 |
| AB016902 | HGC6.3 | | 8130824 |
| NM_000413 | HSD17B1 | 00140, 01100 | 8007263 |
| NM_001142467 | HES4 | | 7911376 |
| NM_181802 | UBE2C | 4120 | 8063043 |
| NM_001164453 | FAM90A20 | | 8144388 |
| NM_178548 | TFAP2E | | 7900001 |
| NM_032810 | ATAD1 | | 7934870 |
| NM_001003682 | TMEM200B | | 7914232 |
| NM_003013 | SFRP2 | 4310 | 8103254 |
| NM_033178 | DUX4 | | 8098732, |
| | | | 8098740, |
| | | | 8098725, |
| | | | 8104122, |
| | | | 8098730, |
| | | | 8098743 or |
| | | | 7931665 |
| NM_003110 | SP2 | | 8008052 |
| NM_021570 | BARX1 | | 8162472 |
| NR_002798 | NAPSB | | 8038547 |
| NM_001029865 | DBX1 | | 7947129 |
| NM_178138 | LHX3 | | 8165083 |
| NM_005202 | COL8A2 | | 7914880 |
| NM_001069 | TUBB2A | 04145, 04540, | 8116649 or |
| | | 05130 | 8116653 |
| NM_006943 | SOX12 | | 8060334 |
| NR_029485 | MIR15A | | 7971661 |
| NM_000986 | RPL24 | 3010 | 8126450 |
| NM_018158 | SLC4A1AP | | 8041015 |
| NM_012188 | FOXI1 | | 8109901 |
| NM_031917 | ANGPTL6 | | 8033892 |
| NM_006118 | HAX1 | | 7905733 |
| NM_000290 | PGAM2 | 00010, 01100 | 8139276 |
| NM_024888 | LPPR3 | | 8032094 |
| NM_002477 | MYL5 | 04510, 04530, | 8093386 |
| | | 04670, 04810 | |
| NM_138383 | MTSS1L | | 7997135 |
| NM_004435 | ENDOG | 4210 | 8158418 |
| BC008667 | PANK2 | 00770, 01100 | 8060736 |
| NM_174920 | SAMD14 | | 8016615 |
| NM_014581 | OBP2B | | 8180358 |
| NM_176677 | NHLRC4 | | 7991898 |
| NM_031157 | HNRNPA1 | 3040 | 7955890 |
| NR_024420 | LOC389634 | | 7960894 |
| NM_032574 | DPY30 | | 8051387 |
| NM_138705 | CALML6 | 04020, 04070, | 7897026 |
| | | 04114, 04270, | |
| | | 04720, 04722, | |
| | | 04740, 04744, | |
| | | 04910, 04912, | |
| | | 04916, 04970, | |
| | | 04971, 05010, | |
| | | 05214 | |
| BC036197 | MGC39545 | | 7944972 |
| BC036837 | C20orf201 | | 8067812 |
| NM_001111322 | DDX54 | | 7958948 |
| NM_016219 | MAN1B1 | 00510, 01100, | 8159566 |
| | | 04141 | |
| NR_029618 | MIR199A2 | | 7922328 |
| NM_001113324 | TEN1 | | 8010017 |
| NM_015414 | RPL36 | 3010 | 8024966 |
| NM_014183 | DYNLRB1 | | 8062016 |
| NM_004781 | VAMP3 | 04130, 04145 | 7897370 |
| NM_002714 | PPP1R10 | | 8124756, |
| | | | 8178358 or |
| | | | 8179664 |
| NM_020341 | PAK7 | 04012, 04360, | 8064952 |
| | | 04510, 04660, | |
| | | 04810, 05211 | |
| NM_080625 | C20orf160 | | 8061653 |
| NM_003969 | UBE2M | 4120 | 8039805 |
| NM_006232 | POLR2H | 00230, 00240, | 8084488 |
| | | 01100, 03020, | |
| | | 05016 | |
| NM_002900 | RBP3 | | 7933359 |
| NM_025147 | COQ10B | | 8047217 |
| NM_016932 | SIX2 | | 8051949 |
| NM_012210 | TRIM32 | 4120 | 8157516 |
| NM_019612 | IRGC | | 8029318 |
| NM_013234 | EIF3K | | 8028514 |
| NM_178351 | LCE1C | | 7920193 |
| NM_007167 | ZMYM6 | | 7914764 |
| NM_004278 | PIGL | 00563, 01100 | 8005157 |
| NM_014688 | USP6NL | | 7926150 |
| NM_006563 | KLF1 | | 8034578 |
| NM_207305 | FOXD4 | | 8159808 |
| NM_001012710 | KRTAP5-10 | | 7942267 |
| NM_020415 | RETN | | 8025278 |
| NM_005225 | E2F1 | 04110, 05200, | 8065710 |
| | | 05212, 05214, | |
| | | 05215, 05218, | |
| | | 05219, 05220, | |
| | | 05222, 05223 | |
| NM_020839 | WDR48 | | 8078834 |
| NM_003332 | TYROBP | 04380, 04650 | 8036224 |
| NM_003396 | WNT9B | 04310, 04340, | 8007895 |
| | | 04916, 05200, | |
| | | 05217 | |
| NR_026811 | AGSK1 | | 7985571 |
| NM_024095 | ASB8 | | 7962783 |
| NM_001330 | CTF1 | 04060, 04630 | 7994961 |
| NM_080865 | GPR62 | | 8080158 |
| NM_144582 | TEX261 | | 8042566 |
| NM_032328 | EFCAB2 | | 7925585 |
| NM_032772 | ZNF503 | | 7928529 |
| NM_000194 | HPRT1 | 00230, 00983, | 8169984 |
| | | 01100 | |
| NM_001009606 | HS3ST6 | | 7998634 |
| NM_001100119 | XRCC3 | 3440 | 7981447 |
| NR_029703 | MIR150 | | 8038393 |
| NM_057176 | BSND | | 7901691 |
| NR_003008 | SCARNA5 | | 8049297 |
| NM_015913 | TXNDC12 | 480 | 7916120 |
| NM_006612 | KIF1C | | 8004057 |
| NM_002798 | PSMB6 | 3050 | 8003953 |
| NM_000684 | ADRB1 | 04020, 04080, | 7930627 |
| | | 04144, 04540, | |
| | | 04970, 05414 | |
| NM_024812 | BAALC | | 8147756 |
| NM_175078 | KRT77 | | 7963502 |
| NM_144736 | C2orf56 | | 8041495 |
| NM_002460 | IRF4 | | 8116559 |
| NM_001001520 | HDGFRP2 | | 8024864 |
| NM_001168479 | ARMCX5 | | 8168958 |
| NR_029596 | MIR129-1 | | 8135907 |
| NM_005565 | LCP2 | 04380, 04650, | 8115734 |
| | | 04660, 04664 | |
| NM_001099279 | FOXD4L2 | | 8161571, |
| | | | 8155440 or |
| | | | 8161583 |
| NM_198595 | AFAP1 | | 8094030 |
| NM_002676 | PMM1 | 00051, 00520, | 8076355 |
| | | 01100 | |
| NR_003594 | REXO1L2P | | 8151603, |
| | | | 8151607, |
| | | | 8151623, |
| | | | 8151619 or |
| | | | 8151627 |
| NM_006427 | SIVA1 | | 7977288 |
| NM_032152 | PRAM1 | | 8033587 |
| NM_001144995 | CCDC85C | | 7981273 |
| NR_002449 | SNORA65 | | 8164215 |
| NM_001002909 | GPATCH8 | | 8016077 |
| NM_016558 | SCAND1 | | 8066031 |
| NM_024335 | IRX6 | | 7995674 |
| NM_004788 | UBE4A | 4120 | 7944195 |
| NM_021247 | PRM3 | | 7999431 |
| NR_024368 | FLJ45340 | | 7945344 |
| AK093358 | FAM27A | | 8161442 |
| NM_033467 | MMEL1 | | 7911767 |
| NR_002937 | C14orf19 | | 7973900 |
| NM_182973 | TMPRSS9 | | 8024467 |
| NM_018663 | PXMP2 | 4146 | 7967789 |
| NM_173728 | ARHGEF15 | | 8004842 |
| NM_004610 | TCP10 | | 8123374 |
| NM_006013 | RPL10 | 3010 | 8034416 or |
| | | | 8109821 |
| NM_015999 | ADIPOR1 | 4920 | 7923503 |
| NM_020209 | SHD | | 8024808 |
| NR_029829 | MIR194-2 | | 7949275 |
| NM_198998 | AQP12A | | 8060126 |
| NM_002233 | KCNA4 | | 7947270 |
| NM_022098 | XPNPEP3 | | 8073311 |
| NM_212550 | BLOC1S3 | | 8029640 |
| NM_198949 | NUDT1 | | 8131101 |
| NM_006626 | ZBTB6 | | 8163999 |
| NM_003795 | SNX3 | | 8128683 |
| NR_029663 | MIR15B | | 8083737 |
| NM_207397 | CD164L2 | | 7914084 |
| NM_014280 | DNAJC8 | | 7899422 |
| NM_001001915 | OR2G2 | 4740 | 7911207 |
| NM_004930 | CAPZB | | 7913169 |
| NM_015950 | MRPL2 | | 8126512 |
| NM_015509 | NECAP1 | | 7953715 |
| NM_006894 | FMO3 | 982 | 7907249 |
| NM_012483 | GNLY | | 8043236 |
| NM_001004333 | RNASEK | | 8004237 |
| NM_147196 | TMIE | | 8079426 |
| NR_027850 | MTX2 | | 8046573 |
| NM_001037283 | EIF3B | 3013 | 8131111 |
| NM_178463 | C20orf166 | | 8063914 |
| BC090923 | C17orf90 | | 8019238 |
| NM_020825 | CRAMP1L | | 7992302 |
| NM_001080520 | DRGX | | 7933501 |
| NM_153232 | EID2 | | 8036749 |
| NM_012186 | FOXE3 | | 7901309 |
| NM_213605 | ZNF517 | | 8148932 |
| NM_178456 | C20orf85 | | 8063601 |
| NM_032547 | SCOC | | 8097521 |
| NM_001170738 | IQSEC3 | 4144 | 8067820 |
| NM_024591 | CHMP6 | 4144 | 8010550 |
| NM_014515 | CNOT2 | 3018 | 7957106 |
| NM_138387 | G6PC3 | | 8007561 |
| NM_024326 | FBXL15 | | 7930099 |
| NM_007262 | PARK7 | 5012 | 7897404 |
| NR_001527 | TTTY6 | | 8176782 or |
| | | | 8177347 |
| NM_001142588 | NFYC | 4612 | 7915345 |
| NM_001080489 | GLOD5 | | 8167356 |
| NM_199243 | GPR150 | | 8106976 |
| NM_000729 | CCK | | 8086391 |
| NM_024718 | C9orf86 | | 8159415 |
| NM_145729 | MRPL24 | | 7921121 |
| NM_030662 | MAP2K2 | 04010, 04012, | 8032761 |
| | | 04270, 04370, | |
| | | 04540, 04620, | |
| | | 04650, 04660, | |
| | | 04662, 04664, | |
| | | 04720, 04722, | |
| | | 04730, 04810, | |
| | | 04910, 04912, | |
| | | 04916, 05020, | |
| | | 05200, 05211, | |
| | | 05213, 05214, | |
| | | 05215, 05216, | |
| | | 05218, 05219, | |
| | | 05220, 05221, | |
| | | 05223 | |
| NM_058190 | FAM207A | | 8180370 |
| NM_001011 | RPS7 | 3010 | 8005877 |
| NM_014336 | AIPL1 | | 8011912 |
| NM_178354 | LCE1F | | 7905517 |
| NM_005470 | ABI1 | | 7932616 |
| NM_006357 | UBE2E3 | 04120, 04141 | 8166124 |
| NR_002182 | NACAP1 | | 8147693 |
| NM_001109763 | GSG1L | | 8000467 |
| NM_001085365 | MZT2A | | 8055287 |
| NM_001080468 | SYCN | | 8036699 |
| NM_003365 | UQCRC1 | 00190, 01100, | 8087100 |
| | | 04260, 05010, | |
| | | 05012, 05016 | |
| NM_153207 | AEBP2 | | 7954279 |
| NM_032477 | MRPL41 | | 8159687 |
| NM_152911 | PAOX | 4146 | 7931582 |
| NM_002488 | NDUFA2 | 00190, 01100, | 8114618 |
| | | 05010, 05012, | |
| | | 05016 | |
| NM_175895 | C12orf61 | | 7964642 |
| NM_004558 | NRTN | | 8024995 |
| NM_198850 | PHLDB3 | | 8037315 |
| NR_023343 | RNU4ATAC | | 8044961 |
| NM_004976 | KCNC1 | | 7938738 |
| NM_012184 | FOXD4L1 | | 8044634 |
| NM_054028 | SLC35G5 | | 8020162 |
| NM_006985 | NPIP | | 7999766 |
| NM_138334 | JOSD2 | | 8038571 |
| NM_014613 | FAF2 | | 8110169 |
| NM_022466 | IKZF5 | | 7936826 |
| NM_022731 | NUCKS1 | | 7909142 |
| NM_138284 | IL17D | | 7967969 |
| NM_004851 | NAPSA | 4142 | 8038556 |
| NM_031292 | PUS7L | | 7962427 |
| NM_017871 | CPSF3L | | 7911486 |
| NM_012476 | VAX2 | | 8042532 |
| NM_032343 | CHCHD6 | | 8082305 |
| NM_199287 | CCDC137 | | 8010629 |
| BC043417 | TMEM68 | | 8146480 |
| NR_029714 | MIR320A | | 8149705 |
| NM_207163 | LMOD2 | | 8135821 |
| NM_024339 | THOC6 | 3013 | 7992795 |
| NM_032411 | C2orf40 | | 8044143 |
| NM_016491 | MRPL37 | | 7901601 |
| NM_003089 | SNRNP70 | 3040 | 8030199 |
| NM_014360 | NKX2-8 | | 7978686 |
| NM_201589 | MAFA | 04930, 04950 | 8153409 |
| AK289373 | IGHG1 | | 8001104 |
| NM_005583 | LYL1 | | 8034608 |
| NM_022375 | OCLM | | 7908347 |
| NM_001006610 | SIAH1 | 04115, 04120, | 8001306 |
| | | 04310 | |
| NM_172229 | KREMEN2 | | 7992758 |
| NM_021996 | GBGT1 | 00603, 01100 | 8164833 |
| NM_194249 | DND1 | | 8114625 |
| NM_001142864 | PIEZO1 | | 7997827 |
| NM_178438 | LCE5A | | 7905483 |
| NM_001349 | DARS | 970 | 8055445 |
| NM_176806 | MOCS2 | 4122 | 8112020 |
| NR_024355 | BK250D10.8 | | 8073546 |
| NM_024768 | CCDC48 | | 8082465 |
| AK093505 | SPANXA2-OT1 | | 8175537 |
| NM_024754 | PTCD2 | | 8106107 |
| AK125905 | LOC100129581 | | 8050113 |
| NM_175619 | ZAR1 | | 8094968 |
| NM_001867 | COX7C | 00190, 01100, | 8106776 |
| | | 04260, 05010, | |
| | | 05012, 05016 | |
| NM_031492 | RBM4B | | 7949674 |
| NM_153376 | CCDC96 | | 8099242 |
| NM_002034 | FUT5 | 00601, 01100 | 8033064 |
| NM_181462 | MRPL55 | | 7924853 |
| NM_012257 | HBP1 | | 8135392 |
| NR_027780 | HMGXB4 | | 8072645 |
| NM_004822 | NTN1 | 4360 | 8004880 |
| NM_178454 | DRAM2 | | 7918474 |
| NM_015893 | PRLH | | 8049509 |
| NM_024313 | NOL12 | | 8072883 |
| NM_006522 | WNT6 | 04310, 04340, | 8048445 |
| | | 04916, 05200, | |
| | | 05217 | |
| NM_017832 | FAM206A | | 8157144 |
| NM_006947 | SRP72 | 3060 | 8095230 |
| NM_021948 | BCAN | | 7906205 |
| NM_001164405 | BHLHA9 | | 8003633 |
| NM_032830 | CIRH1A | 3008 | 7996891 |
| NM_080680 | COL11A2 | 04510, 04512, | 8125568 |
| | | 04974, 05146 | |
| NM_002622 | PFDN1 | | 8114567 |
| NM_002196 | INSM1 | | 8061303 |
| NM_002370 | MAGOH | 03013, 03015, | 7916274 |
| | | 03040 | |
| NR_024406 | LOC732275 | | 8003230 |
| NM_201653 | CHIA | 520 | 7903945 |
| AK296222 | LOC728093 | | 8112476 |
| NM_004343 | CALR | 04141, 04145, | 8026106 |
| | | 04612, 05142 | |
| NM_001003892 | DUPD1 | | 7934527 |
| NR_026837 | LOC283392 | | 7964976 |
| NM_006274 | CCL19 | 04060, 04062 | 8160879 |
| NM_014847 | UBAP2L | | 7905700 |
| NM_016396 | CTDSPL2 | | 7983335 |
| NM_015944 | AMDHD2 | 520 | 7992656 |
| NM_144567 | ANGEL2 | | 7924190 |
| NM_153270 | KLHL34 | | 8171786 |
| NM_080622 | ABHD16B | | 8064203 |
| NM_022753 | S100PBP | | 7899829 |
| AK290103 | LOC100287934 | | 7909990 |
| NR_000011 | SNORA70 | | 8170863 |
| NM_003968 | UBA3 | 4120 | 8088718 |
| NM_033257 | DGCR6L | | 8074565 |
| NM_016468 | COX16 | | 7979906 |
| NM_030642 | APOL5 | | 8072721 |
| NM_002565 | P2RY4 | 4080 | 8173366 |
| NM_014017 | LAMTOR2 | | 7906072 |
| NM_006192 | PAX1 | | 8061357 |
| NM_133450 | ANKS3 | | 7999177 |
| NM_024302 | MMP28 | | 8014282 |
| NM_016166 | PIAS1 | 04120, 04630, | 7984453 |
| | | 05160, 05200, | |
| | | 05222 | |
| NM_007037 | ADAMTS8 | | 7952752 |
| NR_033192 | CCDC59 | | 7965200 |
| NM_174895 | PCP2 | | 8033414 |
| NM_003279 | TNNC2 | 4020 | 8066590 |
| NM_001136262 | ATXN7L3B | | 7957242 |
| NM_004108 | FCN2 | | 8159211 |
| NM_032829 | C12orf34 | | 7958577 |
| NM_139136 | KCNC2 | | 7964987 |
| NM_033113 | ZNF628 | | 8031489 |
| NM_005687 | FARSB | 970 | 8059319 |
| NM_000180 | GUCY2D | 00230, 04740, | 8004763 |
| | | 04744 | |
| NM_024309 | TNIP2 | | 8099029 |
| NM_014554 | SENP1 | | 7962760 |
| NM_001013735 | FOXB2 | | 8155942 |
| NM_174922 | ADCK5 | | 8148850 |
| NM_032704 | TUBA1C | 04145, 04540, | 7955179 |
| | | 05130 | |
| NM_020385 | REXO4 | | 8164907 |
| NR_029894 | MIR148B | | 7955906 |
| NM_001040425 | U2AF1L4 | | 8036143 |
| NR_029945 | MIR423 | | 8006119 |
| U63828 | C20orf181 | | 8067754 |
| NM_133636 | HELQ | | 8101467 |
| NM_001102614 | SLC35G6 | | 8004428 |
| NM_004549 | NDUFC2 | 00190, 01100, | 7950644 |
| | | 05010, 05012, | |
| | | 05016 | |
| AY358101 | DBF4B | | 8007673 |
| NM_032842 | TMEM209 | | 8142912 |
| NM_001862 | COX5B | 00190, 01100, | 8043718 |
| | | 04260, 05010, | |
| | | 05012, 05016 | |
| NM_020535 | KIR2DL5A | 04612, 04650, | 8039884 |
| | | 05332 | |

In another embodiment, cycling hypoxia markers are selected from the list of the 298 cycling hypoxia markers of Table 3 below, as well as their variants, fragments or equivalents. Table 3 comprises cycling hypoxia markers identified in the conditions of the Example and presenting an average FDR corrected p-value over 200 data resampling lower than 0.05.

TABLE 3

| GeneBank | Name of the | | |
|---|---|---|---|
| Accession Number | marker | Pathways | Probeset |
| NR_002312 | RPPH1 | | 7977507 |
| BC018448 | MALAT1 | | 7949410 |
| AF284753 | UIMC1 | | 8165703, or |
| | | | 7911343 |
| NM_014248 | RBX1 | 03420, 04110, | 8073334 |
| | | 04114, 04120, | |
| | | 04141, 04310, | |
| | | 04350, 04710, | |
| | | 05200, 05211 | |
| NR_003287 | RN28S1 | | 7942875 |
| NM_177987 | TUBB8 | 04145, 04540, | 7911355 |
| | | 05130 | |
| NM_170601 | SIAE | | 7944867 |
| NM_001012708 | KRTAP5-3 | | 7945652 |
| NR_029710 | MIR193A | | 8006321 |
| NM_000981 | RPL19 | 3010 | 8006845 |
| NM_003792 | EDF1 | | 8165309 |
| NM_012217 | TPSD1 | | 7992191 |
| NR_029824 | MIR128-2 | | 8078527 |
| NM_003731 | SSNA1 | | 8159609 |
| BC013044 | DNAJA2 | 4141 | 7995379 |
| NM_001037160 | CYS1 | | 8050232 |
| NM_004352 | CBLN1 | | 8001329 |
| NM_006160 | NEUROD2 | | 8014865 |
| NM_001017 | RPS13 | 3010 | 7946812 |
| NM_017854 | TMEM160 | | 8037853 |
| NM_152568 | NKX6-3 | | 8150433 |
| NM_001417 | EIF4B | 03013, 04150 | 7963575 |
| NM_003094 | SNRPE | 3040 | 8160033 |
| NR_002715 | RN7SL1 | | 8040338 |
| NM_016170 | TLX2 | | 8042896 |
| NM_000307 | POU3F4 | | 8168567 |
| NM_016057 | COPZ1 | | 7955896 |
| NM_012322 | LSM5 | 03018, 03040 | 8138912 |
| AK302042 | LOC440518 | | 8027343 |
| NM_000863 | HTR1B | 4080 | 8127692 |
| NM_003512 | HIST1H2AC | 5322 | 8117372 |
| NM_001080113 | C14orf184 | | 7980859 |
| NR_024583 | POM121L8P | | 8071168 |
| NM_145232 | CTU1 | 4122 | 8038782 |
| AK123383 | LOC642648 | | 8076747 |
| NM_032479 | MRPL36 | | 8110861 |
| NM_007241 | SNF8 | 4144 | 8016508 |
| NM_016564 | CEND1 | | 7945536 |
| NR_033335 | SNORA70G | | 7964830 |
| NM_144615 | TMIGD2 | | 8032782 |
| NM_003538 | HIST1H4A | 5322 | 8117334 |
| NM_016424 | LUC7L3 | | 8016733 |
| NM_032231 | FAM96A | | 7989611 |
| NM_014206 | C11orf10 | | 7948606 |
| NR_000009 | SNORD4B | | 8005957 |
| NM_001135086 | PRSS41 | | 7992716 |
| AK125166 | LOC441268 | | 8141166 |
| NR_001445 | RN7SK | | 8120249 |
| NM_001551 | IGBP1 | | 8168087 |
| NM_005608 | PTPRCAP | | 7949792 |
| NM_145203 | CSNK1A1L | 04310, 04340 | 7971071 |
| NM_023002 | HAPLN4 | | 8035646 |
| NM_003542 | HIST1H4C | 5322 | 8117368 |
| NM_001001521 | UGP2 | 00040, 00052, | 8052624 |
| | | 00500, 00520, | |
| | | 01100 | |
| NM_031210 | SLIRP | | 7975989 |
| NM_013299 | SAC3D1 | | 7941122 |
| NM_001044370 | MPPED1 | | 8073623 |
| NM_004175 | SNRPD3 | 03040, 05322 | 8071920 |
| NM_006327 | TIMM23 | | 7927548 |
| NM_031213 | FAM108A1 | | 8032371 |
| NM_001031 | RPS28 | 3010 | 8025395, |
| | | | 8005471, or |
| | | | 7942824 |
| BC033986 | LOC440934 | | 8048712 |
| BC005079 | C2orf42 | | 8052834 |
| BC001181 | FAM173A | | 7992043 |
| NM_000847 | GSTA3 | 00480, 00980, | 8127087 |
| | | 00982 | |
| NM_017900 | AURKAIP1 | | 8039923 or |
| | | | 7911532 |
| NM_001018138 | NM_E2 | 00230, 00240, | 8180388, |
| | | 01100 | 8180389, |
| | | | 8180387 or |
| | | | 8180386 |
| NM_001082575 | RBFOX3 | | 8018993 |
| NM_015456 | COBRA1 | | 8159654 |
| NM_080603 | ZSWIM1 | | 8063074 |
| NM_001029 | RPS26 | 3010 | 8007797 |
| NM_006088 | TUBB2C | 04145, 04540, | 8165496 |
| | | 05130 | |
| NM_004609 | TCF15 | | 8064370 |
| NM_181887 | UBE2D3 | 04120, 04141 | 8180330, |
| | | | 8180335, |
| | | | 8180331, |
| | | | 8180334, |
| | | | 8180333, |
| | | | 8180329 or |
| | | | 8180332 |
| NM_015965 | NDUFA13 | 00190, 05010, | 8027205 |
| | | 05012, 05016 | |
| NM_178536 | LCN12 | | 8159501 |
| NM_032753 | RAX2 | | 8032601 |
| NM_016060 | MED31 | | 8011968 |
| NR_003666 | SPDYE7P | | 8133209 |
| AK125308 | LOC100129484 | | 8137962 |
| NR_003051 | RMRP | | 8161024 |
| NM_005319 | HIST1H1C | | 8124397 |
| NM_003550 | MAD1L1 | 04110, 04914 | 8137805 |
| ENST00000427835 | C20orf61 | | 8065013 |
| NM_144999 | LRRC45 | | 8010719 |
| NM_006087 | TUBB4 | 04145, 04540, | 8025051 |
| | | 05130 | |
| NR_029583 | MIR197 | | 7903717 |
| NM_002307 | LGALS7 | | 8036584 or |
| | | | 8028546 |
| NM_014370 | SRPK3 | | 8170753 |
| AK098732 | TRAP1 | | 7992954 |
| NM_001348 | DAPK3 | 05200, 05219 | 8032718 |
| NM_022061 | MRPL17 | | 7946267 |
| NM_002528 | NTHL1 | 3410 | 7998692 |
| NM_032527 | ZGPAT | | 8064156 |
| NM_006858 | TMED1 | | 8034101 |
| NM_005274 | GNG5 | 4062 | 8174509 |
| NM_022363 | LHX5 | | 7966631 |
| NM_001037495 | DYNLL1 | 4962 | 7967067 |
| NM_021104 | RPL41 | 3010 | 7957530 |
| AK095987 | FLJ38668 | | 8054449 |
| NM_181838 | UBE2D2 | 04120, 04141, | 8108435 |
| | | 05131 | |
| NM_002003 | FCN1 | | 8165011 |
| NM_016589 | TIMMDC1 | | 8081867 |
| NM_020412 | CHMP1B | 4144 | 8020179 |
| NR_026800 | KIAA0125 | | 7977440 |
| NM_014674 | EDEM1 | 4141 | 8085116 |
| NM_138417 | KTI12 | | 7916130 |
| NM_012315 | KLK9 | | 8038716 |
| NR_029681 | MIR140 | | 7997008 |
| NM_173547 | TRIM65 | | 8018502 |
| NM_003577 | UTF1 | | 7931553 |
| NM_138574 | HDGFL1 | | 8117172 |
| NM_018942 | HMX1 | | 8104136 |
| NM_001080495 | TNRC18 | | 8137959 |
| NM_002066 | GML | | 8148565 |
| NM_001168 | BIRC5 | 05200, 05210 | 8018860 |
| NM_020064 | BARHL1 | | 8158912 |
| NM_021012 | KCNJ12 | | 8005726 |
| NM_001007595 | C2CD4B | | 7989473 |
| NM_031899 | GORASP1 | | 8086317 |
| NM_019082 | DDX56 | | 8139392 |
| AY730278 | CENPVL1 | | 8172715 or |
| | | | 8167652 |
| NM_175064 | SPDYE1 | | 8140424 or |
| | | | 8140454 |
| NM_001033113 | ENTPD8 | 00230, 00240 | 8165538 |
| NM_006770 | MARCO | 4145 | 8044773 |
| AK094921 | LOC100131763 | | 8049950 |
| NM_001126128 | PROK2 | | 8088813 |
| NM_016063 | HDDC2 | | 8129363 |
| NM_182532 | TMEM61 | | 7901687 |
| NR_024591 | POM121L1P | | 8074714 |
| NM_020180 | CELF4 | | 8022952 |
| NM_012394 | PFDN2 | | 7921786 |
| NM_006312 | NCOR2 | 4330 | 7959772 |
| NM_001804 | CDX1 | | 8109226 |
| NR_026676 | RPS2P32 | | 8131869 |
| NM_001002 | RPLP0 | 3010 | 8109750 |
| NR_002781 | TSPY26P | | 8065603 |
| NM_003001 | SDHC | 00020, 00190, | 8011212 |
| | | 01100, 05010, | |
| | | 05012, 05016 | |
| NM_002669 | PLRG1 | 3040 | 8103289 |
| NM_006801 | KDELR1 | 5110 | 8038078 |
| NM_015971 | MRPS7 | | 8009784 |
| NM_003684 | MKNK1 | 04010, 04910 | 7915846 |
| NM_018047 | RBM22 | 3040 | 8115168 |
| NM_012249 | RHOQ | 4910 | 8041808 |
| NM_001172743 | RAI2 | | 8171539 |
| NM_016305 | SS18L2 | | 8079074 |
| NM_001113201 | NACA | | 7964262 |
| NM_001144936 | C11orf95 | | 7949015 |
| NM_012452 | TNFRSF13B | 04060, 04672, | 8013061 |
| | | 05340 | |
| NM_004939 | DDX1 | | 8040386 |
| NM_003493 | HIST3H3 | 5322 | 7924884 |
| NM_001013 | RPS9 | 3010 | 8180398 |
| NM_006356 | ATP5H | 00190, 01100, | 8018288 |
| | | 05010, 05012, | |
| | | 05016 | |
| NM_145657 | GSX1 | | 7968260 |
| NM_182702 | PRSS42 | | 8086683 |
| NM_005574 | LMO2 | | 7947450 |
| NM_003926 | MBD3 | | 8032275 |
| NM_024816 | RABEP2 | | 8000616 |
| NM_003859 | DPM1 | 00510, 01100 | 8067017 |
| NM_022097 | CHP2 | 04010, 04020, | 7994123 |
| | | 04114, 04210, | |
| | | 04310, 04360, | |
| | | 04370, 04650, | |
| | | 04660, 04662, | |
| | | 04720, 05010, | |
| | | 05014 | |
| NM_014582 | OBP2A | | 8180231 |
| L20860 | SEPT5-GP1BB | | 8071272 |
| NM_016602 | CCR10 | 04060, 04062, | 8015681 |
| | | 04672 | |
| NM_006292 | TSG101 | 4144 | 7947015 |
| NM_003910 | BUD31 | 3040 | 8134589 |
| NM_005034 | POLR2K | 00230, 00240, | 8147654 |
| | | 01100, 03020, | |
| | | 05016 | |
| NM_032338 | LLPH | | 7956876 |
| NM_003168 | SUPT4H1 | | 8016982 |
| NM_006181 | NTN3 | 4360 | 7992632 |
| NM_012145 | DTYMK | 00240, 01100 | 8077262 or |
| | | | 8060286 |
| NM_032805 | ZSCAN10 | | 7998921 |
| NR_026716 | KIR3DX1 | | 8031200 |
| NM_016835 | MAPT | 04010, 05010 | 8016263 |
| NM_001080440 | OTOL1 | | 8083770 |
| NM_198180 | QRFP | | 8164630 |
| NM_139172 | TMEM190 | | 8031475 |
| NM_101395 | DYRK1A | | 8068551 |
| NM_003537 | HIST1H3B | 5322 | 8124388 |
| BC043386 | C19orf68 | | 8029996 |
| NM_144589 | COMTD1 | | 7934544 |
| NM_005922 | MAP3K4 | 04010, 04912 | 8130624 |
| NM_018462 | BRK1 | 4810 | 8085287 |
| AK097604 | LOC100130285 | | 7998265 |
| NM_015719 | COL5A3 | 04510, 04512, | 8033825 |
| | | 04974, 05146 | |
| NR_002951 | SNORA2B | | 7962829 |
| NM_001039916 | ZNF384 | | 7953390 |
| AF304442 | C21orf118 | | 8068046 |
| NR_026557 | PLK5 | | 8024331 |
| NM_000377 | WAS | 04062, 04520, | 8167334 |
| | | 04666, 04810, | |
| | | 05100, 05130, | |
| | | 05131 | |
| NM_006848 | CCDC85B | | 7941457 |
| NR_001555 | GOLGA2P2Y | | 8177413 or |
| | | | 8176910 |
| NM_001003684 | UQCR10 | 00190, 01100, | 8072274 |
| | | 04260, 05010, | |
| | | 05012, 05016 | |
| NM_174923 | CCDC107 | | 8155073 |
| NM_014419 | DKKL1 | | 8030292 |
| NM_025072 | PTGES2 | 00590, 01100 | 8164362 |
| NM_001164447 | FAM90A10 | | 8144448 |
| AK125575 | ZNF425 | | 8143708 |
| NM_006808 | SEC61B | 03060, 04141, | 8156838 |
| | | 04145, 05110 | |
| NM_007255 | B4GALT7 | 00532, 00534, | 8110399 |
| | | 01100 | |
| NM_001164440 | ANKRD33B | | 8104499 |
| NM_021646 | ZNF500 | | 7999196 |
| NM_031909 | C1QTNF4 | | 7947928 |
| NM_002297 | LCN1 | | 8159255 |
| NM_001013653 | LRRC26 | | 8165453 |
| NM_014171 | CRIPT | | 8041813 |
| NM_003375 | VDAC2 | 04020, 05012, | 8042335 |
| | | 05016 | |
| NM_003513 | HIST1H2AB | 5322 | 8124391 |
| NM_001024678 | LRRC24 | | 8153868 |
| NR_024593 | POM121L10P | | 8075024 |
| NR_026713 | FAM182A | | 8065527 |
| NM_001144954 | C5orf47 | | 8110068 |
| NM_001099435 | SPDYE5 | | 8133654 |
| NM_175741 | C15orf55 | | 7982516 |
| NM_020199 | C5orf15 | | 8114138 |
| NM_000183 | HADHB | 00062, 00071, | 8040639 |
| | | 00280, 01100 | |
| NM_014860 | SUPT7L | | 8051204 |
| NM_016568 | RXFP3 | | 8104781 |
| NM_014976 | PDCD11 | | 7936096 |
| NR_002576 | SNORA21 | | 8014755 |
| NM_144578 | MAPK1IP1L | | 7974455 |
| NM_006476 | ATP5L | 00190, 01100 | 7944216 |
| NM_130784 | SYCE1 | | 7937247 |
| NM_006043 | HS3ST2 | 534 | 7994052 |
| NR_002144 | LOC407835 | | 8136065 |
| NM_001800 | CDKN2D | 4110 | 8034075 |
| NM_005007 | NFKBIL1 | | 8179249, |
| | | | 8177967 or |
| | | | 8118127 |
| NM_003278 | CLEC3B | | 8079305 |
| NM_002494 | NDUFC1 | 00190, 01100, | 8102839 |
| | | 05010, 05012, | |
| | | 05016 | |
| NM_006855 | KDELR3 | 5110 | 8073015 |
| BC004943 | MGC10814 | | 8035551 |
| NM_019107 | C19orf10 | | 8032863 |
| NM_145803 | TRAF6 | 04010, 04120, | 7947540 |
| | | 04144, 04380, | |
| | | 04620, 04621, | |
| | | 04622, 04722, | |
| | | 05140, 05142, | |
| | | 05145, 05160, | |
| | | 05200, 05222 | |
| NR_003013 | SCARNA16 | | 8010137 |
| NM_014170 | GTPBP8 | | 8081676 |
| NM_016438 | HIGD1B | | 8007701 |
| NM_016199 | LSM7 | 03018, 03040 | 8032480 |
| NM_004637 | RAB7A | 04144, 04145, | 8082431 |
| | | 05146 | |
| NM_015679 | TRUB2 | | 8164428 |
| NM_138983 | OLIG1 | | 8068235 |
| NM_005706 | TSSC4 | | 7937813 |
| NM_003002 | SDHD | 00020, 00190, | 7943853 |
| | | 01100, 05010, | |
| | | 05012, 05016 | |
| NM_016734 | PAX5 | | 8161211 |
| NM_001164456 | FAM90A13 | | 8149204 |
| NM_014064 | METTL11A | | 8158544 |
| NM_006686 | ACTL7B | | 8163019 |
| NM_001258 | CDK3 | | 8010021 |
| NM_030811 | MRPS26 | | 8060599 |
| NM_133261 | GIPC3 | | 8024676 |
| AF067420 | IGHA1 | | 7995263 |
| NM_002764 | PRPS1 | 00030, 00230, | 8169240 |
| | | 01100 | |
| NM_152898 | FERD3L | | 8138450 |
| NM_015276 | USP22 | | 8013486 |
| NM_144727 | CRYGN | | 8143949 |
| NM_001037984 | SLC38A10 | | 8019149 |
| NM_002804 | PSMC3 | 3050 | 7947867 |
| NM_018250 | INTS9 | | 8150014 |
| NM_033644 | FBXW11 | 04114, 04120, | 8115765 |
| | | 04310, 04340, | |
| | | 04710, 05131 | |
| NM_021570 | BARX1 | | 8162472 |
| NM_007374 | SIX6 | | 7974793 |
| NR_003502 | ZNRF2P1 | | 8132209 |
| AY341951 | FAM138D | | 7960172 |
| NM_004640 | DDX39B | 03013, 03015, | 8178476 or |
| | | 03040 | 8179750 |
| BC104424 | FAHD2B | | 8043682 |
| NM_000383 | AIRE | 04120, 05340 | 8069037 |
| NM_001164453 | FAM90A20 | | 8144388 |
| NM_004855 | PIGB | 00563, 01100 | 7983811 |
| NM_001145250 | SP9 | | 8056825 |
| NM_178138 | LHX3 | | 8165083 |
| NM_152914 | C17orf103 | | 8013509 |
| NM_173660 | DOK7 | | 8093807 |
| NM_006299 | ZNF193 | | 8117655 |
| NM_182498 | ZNF428 | | 8037355 |
| NM_148172 | PEMT | 00564, 01100 | 8013120 |
| AB016902 | HGC6.3 | | 8130824 |
| NM_002949 | MRPL12 | | 8010664 |
| NR_002911 | SNORA71A | | 8066258 |
| NM_005091 | PGLYRP1 | | 8037742 |
| AK291454 | UBE2K | 4120 | 8099918 |
| NM_016312 | WBP11 | 3040 | 7961489 |
| NM_004435 | ENDOG | 4210 | 8158418 |
| NM_173514 | SLC38A9 | | 8112121 |
| NM_014342 | MTCH2 | | 7947934 |
| NM_152778 | MFSD8 | 4142 | 8102730 |
| NR_004430 | RNU1-1 | | 7919349 or |
| | | | 7919269 |
| NM_001135580 | C19orf71 | | 8024655 |
| NM_014581 | OBP2B | | 8180358 |
| NM_000479 | AMH | 04060, 04350 | 8024429 |
| NM_001164094 | COPS7A | | 7953395 |
| NM_001001410 | C16orf42 | | 7998449 |
| NM_001005922 | KRTAP5-1 | | 7945645 |
| NM_003013 | SFRP2 | 4310 | 8103254 |
| NM_052945 | TNFRSF13C | 04060, 04672, | 8076387 |
| | | 05340 | |
| NM_000290 | PGAM2 | 00010, 01100 | 8139276 |
| NM_001100418 | C19orf60 | | 8027032 |
| NM_002714 | PPP1R10 | | 8179664 or |
| | | | 8178358 |
| NM_015568 | PPP1R16B | | 8062557 |
| NM_020070 | IGLL1 | 5340 | 8074909 |
| NM_012188 | FOXI1 | | 8109901 |

In another embodiment, cycling hypoxia markers are selected from the list of the 167 cycling hypoxia markers of Table 4 below, as well as their variants, fragments or equivalents. Table 4 comprises cycling hypoxia markers identified in the conditions of the Example and presenting an average FDR corrected p-value over 200 data resampling lower than 0.01.

TABLE 4

| GeneBank | Name of the | | |
|---|---|---|---|
| Accession Number | marker | Pathways | Probeset |
| NR_002312 | RPPH1 | | 7977507 |
| BC018448 | MALAT1 | | 7949410 |
| AF284753 | UIMC1 | | 8165703 or |
| | | | 7911343 |
| NM_014248 | RBX1 | 03420, 04110, | 8073334 |
| | | 04114, 04120, | |
| | | 04141, 04310, | |
| | | 04350, 04710, | |
| | | 05200, 05211 | |
| NR_003287 | RN28S1 | | 7942875 |
| NM_177987 | TUBB8 | 04145, 04540, | 7911355 |
| | | 05130 | |
| NM_170601 | SIAE | | 7944867 |
| NM_001012708 | KRTAP5-3 | | 7945652 |
| NR_029710 | MIR193A | | 8006321 |
| NM_000981 | RPL19 | 3010 | 8006845 |
| NM_003792 | EDF1 | | 8165309 |
| NM_012217 | TPSD1 | | 7992191 |
| NR_029824 | MIR128-2 | | 8078527 |
| NM_003731 | SSNA1 | | 8159609 |
| BC013044 | DNAJA2 | 4141 | 7995379 |
| NM_001037160 | CYS1 | | 8050232 |
| NM_004352 | CBLN1 | | 8001329 |
| NM_006160 | NEUROD2 | | 8014865 |
| NM_001017 | RPS13 | 3010 | 7946812 |
| NM_017854 | TMEM160 | | 8037853 |
| NM_152568 | NKX6-3 | | 8150433 |
| NM_001417 | EIF4B | 03013, 04150 | 7963575 |
| NM_003094 | SNRPE | 3040 | 8160033 |
| NR_002715 | RN7SL1 | | 8040338 |
| NM_016170 | TLX2 | | 8042896 |
| NM_000307 | POU3F4 | | 8168567 |
| NM_016057 | COPZ1 | | 7955896 |
| NM_012322 | LSM5 | 03018, 03040 | 8138912 |
| AK302042 | LOC440518 | | 8027343 |
| NM_000863 | HTR1B | 4080 | 8127692 |
| NM_003512 | HIST1H2AC | 5322 | 8117372 |
| NM_001080113 | C14orf184 | | 7980859 |
| NR_024583 | POM121L8P | | 8071168 |
| NM_145232 | CTU1 | 4122 | 8038782 |
| AK123383 | LOC642648 | | 8076747 |
| NM_032479 | MRPL36 | | 8110861 |
| NM_007241 | SNF8 | 4144 | 8016508 |
| NM_016564 | CEND1 | | 7945536 |
| NR_033335 | SNORA70G | | 7964830 |
| NM_144615 | TMIGD2 | | 8032782 |
| NM_003538 | HIST1H4A | 5322 | 8117334 |
| NM_016424 | LUC7L3 | | 8016733 |
| NM_032231 | FAM96A | | 7989611 |
| NM_014206 | C11orf10 | | 7948606 |
| NR_000009 | SNORD4B | | 8005957 |
| NM_001135086 | PRSS41 | | 7992716 |
| AK125166 | LOC441268 | | 8141166 |
| NR_001445 | RN7SK | | 8120249 |
| NM_001551 | IGBP1 | | 8168087 |
| NM_005608 | PTPRCAP | | 7949792 |
| NM_145203 | CSNK1A1L | 04310, 04340 | 7971071 |
| NM_023002 | HAPLN4 | | 8035646 |
| NM_003542 | HIST1H4C | 5322 | 8117368 |
| NM_001001521 | UGP2 | 00040, 00052, | 8052624 |
| | | 00500, 00520, | |
| | | 01100 | |
| NM_031210 | SLIRP | | 7975989 |
| NM_013299 | SAC3D1 | | 7941122 |
| NM_001044370 | MPPED1 | | 8073623 |
| NM_004175 | SNRPD3 | 03040, 05322 | 8071920 |
| NM_006327 | TIMM23 | | 7927548 |
| NM_031213 | FAM108A1 | | 8032371 |
| NM_001031 | RPS28 | 3010 | 8025395, |
| | | | 8005471 or |
| | | | 7942824 |
| BC033986 | LOC440934 | | 8048712 |
| BC005079 | C2orf42 | | 8052834 |
| BC001181 | FAM173A | | 7992043 |
| NM_000847 | GSTA3 | 00480, 00980, | 8127087 |
| | | 00982 | |
| NM_017900 | AURKAIP1 | | 8039923, |
| | | | 7911532 |
| NM_001018138 | NM_E2 | 00230, 00240, | 8180388, |
| | | 01100 | 8180389, |
| | | | 8180387 or |
| | | | 8180386 |
| NM_001082575 | RBFOX3 | | 8018993 |
| NM_015456 | COBRA1 | | 8159654 |
| NM_080603 | ZSWIM1 | | 8063074 |
| NM_001029 | RPS26 | 3010 | 8007797 |
| NM_006088 | TUBB2C | 04145, 04540, | 8165496 |
| | | 05130 | |
| NM_004609 | TCF15 | | 8064370 |
| NM_181887 | UBE2D3 | 04120, 04141 | 8180330, |
| | | | 8180335, |
| | | | 8180331, |
| | | | 8180334, |
| | | | 8180333, |
| | | | 8180329 or |
| | | | 8180332 |
| NM_015965 | NDUFA13 | 00190, 05010, | 8027205 |
| | | 05012, 05016 | |
| NM_178536 | LCN12 | | 8159501 |
| NM_032753 | RAX2 | | 8032601 |
| NM_016060 | MED31 | | 8011968 |
| NR_003666 | SPDYE7P | | 8133209 |
| AK125308 | LOC100129484 | | 8137962 |
| NR_003051 | RMRP | | 8161024 |
| NM_005319 | HIST1H1C | | 8124397 |
| NM_003550 | MAD1L1 | 04110, 04914 | 8137805 |
| ENST00000427835 | C20orf61 | | 8065013 |
| NM_144999 | LRRC45 | | 8010719 |
| NM_006087 | TUBB4 | 04145, 04540, | 8025051 |
| | | 05130 | |
| NR_029583 | MIR197 | | 7903717 |
| NM_002307 | LGALS7 | | 8036584 or |
| | | | 8028546 |
| NM_014370 | SRPK3 | | 8170753 |
| AK098732 | TRAP1 | | 7992954 |
| NM_001348 | DAPK3 | 05200, 05219, | 8032718 |
| NM_022061 | MRPL17 | | 7946267 |
| NM_002528 | NTHL1 | 3410 | 7998692 |
| NM_032527 | ZGPAT | | 8064156 |
| NM_006858 | TMED1 | | 8034101 |
| NM_005274 | GNG5 | 4062 | 8174509 |
| NM_022363 | LHX5 | | 7966631 |
| NM_001037495 | DYNLL1 | 4962 | 7967067 |
| NM_021104 | RPL41 | 3010 | 7957530 |
| AK095987 | FLJ38668 | | 8054449 |
| NM_181838 | UBE2D2 | 04120, 04141, | 8108435 |
| | | 05131 | |
| NM_002003 | FCN1 | | 8165011 |
| NM_016589 | TIMMDC1 | | 8081867 |
| NM_020412 | CHMP1B | 4144 | 8020179 |
| NR_026800 | KIAA0125 | | 7977440 |
| NM_014674 | EDEM1 | 4141 | 8085116 |
| NM_138417 | KTI12 | | 7916130 |
| NM_012315 | KLK9 | | 8038716 |
| NR_029681 | MIR140 | | 7997008 |
| NM_173547 | TRIM65 | | 8018502 |
| NM_003577 | UTF1 | | 7931553 |
| NM_138574 | HDGFL1 | | 8117172 |
| NM_018942 | HMX1 | | 8104136 |
| NM_001080495 | TNRC18 | | 8137959 |
| NM_002066 | GML | | 8148565 |
| NM_001168 | BIRC5 | 05200, 05210 | 8018860 |
| NM_020064 | BARHL1 | | 8158912 |
| NM_021012 | KCNJ12 | | 8005726 |
| NM_001007595 | C2CD4B | | 7989473 |
| NM_031899 | GORASP1 | | 8086317 |
| NM_019082 | DDX56 | | 8139392 |
| AY730278 | CENPVL1 | | 8172715 or |
| | | | 8167652 |
| NM_175064 | SPDYE1 | | 8140424 |
| NM_001033113 | ENTPD8 | 00230, 00240, | 8165538 |
| NM_006770 | MARCO | 4145 | 8044773 |
| AK094921 | LOC100131763 | | 8049950 |
| NM_001126128 | PROK2 | | 8088813 |
| NM_016063 | HDDC2 | | 8129363 |
| NM_182532 | TMEM61 | | 7901687 |
| NR_024591 | POM121L1P | | 8074714 |
| NM_020180 | CELF4 | | 8022952 |
| NM_012394 | PFDN2 | | 7921786 |
| NM_006312 | NCOR2 | 4330 | 7959772 |
| NM_001804 | CDX1 | | 8109226 |
| NR_026676 | RPS2P32 | | 8131869 |
| NM_001002 | RPLP0 | 3010 | 8109750 |
| NR_002781 | TSPY26P | | 8065603 |
| NM_003001 | SDHC | 00020, 00190, | 8011212 |
| | | 01100, 05010, | |
| | | 05012, 05016, | |
| NM_002669 | PLRG1 | 3040 | 8103289 |
| NM_006801 | KDELR1 | 5110 | 8038078 |
| NM_015971 | MRPS7 | | 8009784 |
| NM_003684 | MKNK1 | 04010, 04910 | 7915846 |
| NM_018047 | RBM22 | 3040 | 8115168 |
| NM_012249 | RHOQ | 4910 | 8041808 |
| NM_001172743 | RAI2 | | 8171539 |
| NM_016305 | SS18L2 | | 8079074 |
| NM_001113201 | NACA | | 7964262 |
| NM_001144936 | C11orf95 | | 7949015 |
| NM_012452 | TNFRSF13B | 04060, 04672, | 8013061 |
| | | 05340 | |
| NM_004939 | DDX1 | | 8040386 |
| NM_003493 | HIST3H3 | 5322 | 7924884 |
| NM_001013 | RPS9 | 3010 | 8180398 |
| NM_006356 | ATP5H | 00190, 01100, | 8018288 |
| | | 05010, 05012, | |
| | | 05016 | |
| NM_145657 | GSX1 | | 7968260 |
| NM_182702 | PRSS42 | | 8086683 |
| NM_005574 | LMO2 | | 7947450 |
| NM_003926 | MBD3 | | 8032275 |
| NM_024816 | RABEP2 | | 8000616 |
| NM_003859 | DPM1 | 00510, 01100 | 8067017 |
| NM_022097 | CHP2 | 04010, 04020, | 7994123 |
| | | 04114, 04210, | |
| | | 04310, 04360, | |
| | | 04370, 04650, | |
| | | 04660, 04662, | |
| | | 04720, 05010, | |
| | | 05014 | |
| NM_014582 | OBP2A | | 8180231 |
| L20860 | SEPT5-GP1BB | | 8071272 |
| NM_016602 | CCR10 | 04060, 04062, | 8015681 |
| | | 04672 | |
| NM_006292 | TSG101 | 4144 | 7947015 |
| NM_003910 | BUD31 | 3040 | 8134589 |
| NM_005034 | POLR2K | 00230, 00240, | 8147654 |
| | | 01100, 03020, | |
| | | 05016 | |

In another embodiment, cycling hypoxia markers are selected from the list of the cycling hypoxia markers of Table 5 below, as well as their variants, fragments or equivalents. Table 5 comprises cycling hypoxia markers identified in the conditions of the Example and which are the 100 probe sets with the lowest FDR corrected p-values average over 200 data resampling, corresponding to 96 annoted genes. Table 5 thus comprises 96 cycling hypoxia markers.

TABLE 5

| GeneBank | Name of the | | |
|---|---|---|---|
| Accession Number | marker | Pathways | Probeset |
| NM_001168 | BIRC5 | 05200, 05210 | 8018860 |
| NM_032527 | ZGPAT | | 8064156 |
| NM_012322 | LSM5 | 03018, 03040 | 8138912 |
| NM_012394 | PFDN2 | | 7921786 |
| NM_002003 | FCN1 | | 8165011 |
| NM_001113201 | NACA | | 7964262 |
| NM_005608 | PTPRCAP | | 7949792 |
| NM_006858 | TMED1 | | 8034101 |
| NM_001551 | IGBP1 | | 8168087 |
| NM_001417 | EIF4B | 03013, 04150 | 7963575 |
| NM_005319 | HIST1H1C | | 8124397 |
| NM_031210 | SLIRP | | 7975989 |
| NM_000863 | HTR1B | 4080 | 8127692 |
| NM_000847 | GSTA3 | 00480, 00980, | 8127087 |
| | | 00982 | |
| NM_013299 | SAC3D1 | | 7941122 |
| NM_002528 | NTHL1 | 3410 | 7998692 |
| NM_001044370 | MPPED1 | | 8073623 |
| NM_006160 | NEUROD2 | | 8014865 |
| NM_021012 | KCNJ12 | | 8005726 |
| NM_022363 | LHX5 | | 7966631 |
| NM_017854 | TMEM160 | | 8037853 |
| NM_018942 | HMX1 | | 8104136 |
| NM_014206 | C11orf10 | | 7948606 |
| NM_006770 | MARCO | 4145 | 8044773 |
| NM_006292 | TSG101 | 4144 | 7947015 |
| NM_003577 | UTF1 | | 7931553 |
| NM_032338 | LLPH | | 7956876 |
| NM_003512 | HIST1H2AC | 5322 | 8117372 |
| NM_004352 | CBLN1 | | 8001329 |
| NM_015965 | NDUFA13 | 00190, 05010, | 8027205 |
| | | 05012, 05016 | |
| NM_016170 | TLX2 | | 8042896 |
| NM_017900 | AURKAIP1 | | 7911532 or |
| | | | 8039923 |
| BC001181 | FAM173A | | 7992043 |
| NM_080603 | ZSWIM1 | | 8063074 |
| NM_012217 | TPSD1 | | 7992191 |
| NM_181838 | UBE2D2 | 04120, 04141, | 8108435 |
| | | 05131 | |
| NM_003792 | EDF1 | | 8165309 |
| NM_022061 | MRPL17 | | 7946267 |
| NM_016564 | CEND1 | | 7945536 |
| NM_003731 | SSNA1 | | 8159609 |
| NM_001031 | RPS28 | 3010 | 8005471, 8025395 |
| | | | or 7942824 |
| NM_014370 | SRPK3 | | 8170753 |
| NM_001348 | DAPK3 | 05200, 05219, | 8032718 |
| NM_001037495 | DYNLL1 | 4962 | 7967067 |
| NM_015456 | COBRA1 | | 8159654 |
| NM_003001 | SDHC | 00020, 00190, | 8011212 |
| | | 01100, 05010, | |
| | | 05012, 05016 | |
| NM_016060 | MED31 | | 8011968 |
| NR_026800 | KIAA0125 | | 7977440 |
| NM_007241 | SNF8 | 4144 | 8016508 |
| NM_000307 | POU3F4 | | 8168567 |
| NM_031899 | GORASP1 | | 8086317 |
| BC005079 | C2orf42 | | 8052834 |
| NM_014248 | RBX1 | 03420, 04110, | 8073334 |
| | | 04114, 04120, | |
| | | 04141, 04310, | |
| | | 04350, 04710, | |
| | | 05200, 05211 | |
| NM_003684 | MKNK1 | 04010, 04910 | 7915846 |
| NM_004175 | SNRPD3 | 03040, 05322 | 8071920 |
| NM_031213 | FAM108A1 | | 8032371 |
| NM_003493 | HIST3H3 | 5322 | 7924884 |
| NM_000981 | RPL19 | 3010 | 8006845 |
| NM_001017 | RPS13 | 3010 | 7946812 |
| NM_001144936 | C11orf95 | | 7949015 |
| NM_015971 | MRPS7 | | 8009784 |
| NM_005274 | GNG5 | 4062 | 8174509 |
| NM_005973 | PRCC | | 7906235 |
| NM_020412 | CHMP1B | 4144 | 8020179 |
| NM_005574 | LMO2 | | 7947450 |
| NM_004609 | TCF15 | | 8064370 |
| NM_016057 | COPZ1 | | 7955896 |
| NM_003550 | MAD1L1 | 04110, 04914 | 8137805 |
| NM_003538 | HIST1H4A | 5322 | 8117334 |
| NM_003542 | HIST1H4C | 5322 | 8117368 |
| NR_002312 | RPPH1 | | 7977507 |
| BC018448 | MALAT1 | | 7949410 |
| NM_152568 | NKX6-3 | | 8150433 |
| NR_024583 | POM121L8P | | 8071168 |
| NM_032231 | FAM96A | | 7989611 |
| NM_001080113 | C14orf184 | | 7980859 |
| NM_144615 | TMIGD2 | | 8032782 |
| NM_032479 | MRPL36 | | 8110861 |
| NM_182532 | TMEM61 | | 7901687 |
| NM_138417 | KTI12 | | 7916130 |
| BC033986 | LOC440934 | | 8048712 |
| NM_001082575 | RBFOX3 | | 8018993 |
| NM_032753 | RAX2 | | 8032601 |
| NM_144999 | LRRC45 | | 8010719 |
| NM_002307 | LGALS7 | | 8036584 or |
| | | | 8028546 |
| NR_003666 | SPDYE7P | | 8133209 |
| NM_178536 | LCN12 | | 8159501 |
| ENST00000427835 | C20orf61 | | 8065013 |
| NM_173547 | TRIM65 | | 8018502 |
| NM_033055 | HIAT1 | | 7903294 |
| NM_001007595 | C2CD4B | | 7989473 |
| AK095987 | FLJ38668 | | 8054449 |
| NM_016589 | TIMMDC1 | | 8081867 |
| NM_144589 | COMTD1 | | 7934544 |
| NM_145657 | GSX1 | | 7968260 |
| NM_020180 | CELF4 | | 8022952 |

In another embodiment, cycling hypoxia markers are selected from the list of the 74 cycling hypoxia markers of Table 6 below, as well as their variants, fragments or equivalents. Table 6 comprises cycling hypoxia markers identified in the conditions of the Example and presenting an average FDR corrected p-value over 200 data resampling lower than 0.001.

TABLE 6

| GeneBank | Name of the | | |
|---|---|---|---|
| Accession Number | marker | Pathways | Probeset |
| NR_002312 | RPPH1 | | 7977507 |
| BC018448 | MALAT1 | | 7949410 |
| AF284753 | UIMC1 | | 8165703 or |
| | | | 7911343 |
| NM_014248 | RBX1 | 03420, 04110, | 8073334 |
| | | 04114, 04120, | |
| | | 04141, 04310, | |
| | | 04350, 04710, | |
| | | 05200, 05211 | |
| NR_003287 | RN28S1 | | 7942875 |
| NM_177987 | TUBB8 | 04145, 04540 | 7911355 |
| | | 05130 | |
| NM_170601 | SIAE | | 7944867 |
| NM_001012708 | KRTAP5-3 | | 7945652 |
| NR_029710 | MIR193A | | 8006321 |
| NM_000981 | RPL19 | 3010 | 8006845 |
| NM_003792 | EDF1 | | 8165309 |
| NM_012217 | TPSD1 | | 7992191 |
| NR_029824 | MIR128-2 | | 8078527 |
| NM_003731 | SSNA1 | | 8159609 |
| BC013044 | DNAJA2 | 4141 | 7995379 |
| NM_001037160 | CYS1 | | 8050232 |
| NM_004352 | CBLN1 | | 8001329 |
| NM_006160 | NEUROD2 | | 8014865 |
| NM_001017 | RPS13 | 3010 | 7946812 |
| NM_017854 | TMEM160 | | 8037853 |
| NM_152568 | NKX6-3 | | 8150433 |
| NM_001417 | EIF4B | 03013, 04150 | 7963575 |
| NM_003094 | SNRPE | 3040 | 8160033 |
| NR_002715 | RN7SL1 | | 8040338 |
| NM_016170 | TLX2 | | 8042896 |
| NM_000307 | POU3F4 | | 8168567 |
| NM_016057 | COPZ1 | | 7955896 |
| NM_012322 | LSM5 | 03018, 03040 | 8138912 |
| AK302042 | LOC440518 | | 8027343 |
| NM_000863 | HTR1B | 4080 | 8127692 |
| NM_003512 | HIST1H2AC | 5322 | 8117372 |
| NM_001080113 | C14orf184 | | 7980859 |
| NR_024583 | POM121L8P | | 8071168 |
| NM_145232 | CTU1 | 4122 | 8038782 |
| AK123383 | LOC642648 | | 8076747 |
| NM_032479 | MRPL36 | | 8110861 |
| NM_007241 | SNF8 | 4144 | 8016508 |
| NM_016564 | CEND1 | | 7945536 |
| NR_033335 | SNORA70G | | 7964830 |
| NM_144615 | TMIGD2 | | 8032782 |
| NM_003538 | HIST1H4A | 5322 | 8117334 |
| NM_016424 | LUC7L3 | | 8016733 |
| NM_032231 | FAM96A | | 7989611 |
| NM_014206 | C11orf10 | | 7948606 |
| NR_000009 | SNORD4B | | 8005957 |
| NM_001135086 | PRSS41 | | 7992716 |
| AK125166 | LOC441268 | | 8141166 |
| NR_001445 | RN7SK | | 8120249 |
| NM_001551 | IGBP1 | | 8168087 |
| NM_005608 | PTPRCAP | | 7949792 |
| NM_145203 | CSNK1A1L | 04310, 04340 | 7971071 |
| NM_023002 | HAPLN4 | | 8035646 |
| NM_003542 | HIST1H4C | 5322 | 8117368 |
| NM_001001521 | UGP2 | 00040, 00052, | 8052624 |
| | | 00500, 00520, | |
| | | 01100 | |
| NM_031210 | SLIRP | | 7975989 |
| NM_013299 | SAC3D1 | | 7941122 |
| NM_001044370 | MPPED1 | | 8073623 |
| NM_004175 | SNRPD3 | 03040, 05322 | 8071920 |
| NM_006327 | TIMM23 | | 7927548 |
| NM_031213 | FAM108A1 | | 8032371 |
| NM_001031 | RPS28 | 3010 | 8025395 or |
| | | | 8005471 |
| BC033986 | LOC440934 | | 8048712 |
| BC005079 | C2orf42 | | 8052834 |
| BC001181 | FAM173A | | 7992043 |
| NM_000847 | GSTA3 | 00480, 00980, | 8127087 |
| | | 00982 | |
| NM_017900 | AURKAIP1 | | 8039923 |
| NM_001018138 | NME2 | 00230, 00240, | 8180388 or |
| | | 01100 | 8180389 |
| NM_001082575 | RBFOX3 | | 8018993 |
| NM_015456 | COBRA1 | | 8159654 |
| NM_080603 | ZSWIM1 | | 8063074 |
| NM_001029 | RPS26 | 3010 | 8007797 |
| NM_006088 | TUBB2C | 04145, 04540, | 8165496 |
| | | 05130 | |
| NM_004609 | TCF15 | | 8064370 |
| NM_181887 | UBE2D3 | 04120 04141 | 8180330, 8180335, |
| | | | 8180331, 8180334 |
| | | | or 8180333 |

In another embodiment, cycling hypoxia markers are selected from the list of the 37 cycling hypoxia markers of Table 7 below, as well as their variants, fragments or equivalents. Table 7 comprises cycling hypoxia markers identified in the conditions of the Example and presenting an average FDR corrected p-value over 200 data resampling lower than 0.0001.

TABLE 7

| GeneBank | Name of the | | |
|---|---|---|---|
| Accession Number | marker | Pathways | Probeset |
| NR_002312 | RPPH1 | | 7977507 |
| BC018448 | MALAT1 | | 7949410 |
| AF284753 | UIMC1 | | 8165703 or |
| | | | 7911343 |
| NM_014248 | RBX1 | 03420, 04110, | 8073334 |
| | | 04114, 04120, | |
| | | 04141, 04310, | |
| | | 04350, 04710, | |
| | | 05200, 05211 | |
| NR_003287 | RN28S1 | | 7942875 |
| NM_177987 | TUBB8 | 04145, 04540, | 7911355 |
| | | 05130 | |
| NM_170601 | SIAE | | 7944867 |
| NM_001012708 | KRTAP5-3 | | 7945652 |
| NR_029710 | MIR193A | | 8006321 |
| NM_000981 | RPL19 | 3010 | 8006845 |
| NM_003792 | EDF1 | | 8165309 |
| NM_012217 | TPSD1 | | 7992191 |
| NR_029824 | MIR128-2 | | 8078527 |
| NM_003731 | SSNA1 | | 8159609 |
| BC013044 | DNAJA2 | 4141 | 7995379 |
| NM_001037160 | CYS1 | | 8050232 |
| NM_004352 | CBLN1 | | 8001329 |
| NM_006160 | NEUROD2 | | 8014865 |
| NM_001017 | RPS13 | 3010 | 7946812 |
| NM_017854 | TMEM160 | | 8037853 |
| NM_152568 | NKX6-3 | | 8150433 |
| NM_001417 | EIF4B | 03013, 04150 | 7963575 |
| NM_003094 | SNRPE | 3040 | 8160033 |
| NR_002715 | RN7SL1 | | 8040338 |
| NM_016170 | TLX2 | | 8042896 |
| NM_000307 | POU3F4 | | 8168567 |
| NM_016057 | COPZ1 | | 7955896 |
| NM_012322 | LSM5 | 03018, 03040 | 8138912 |
| AK302042 | LOC440518 | | 8027343 |
| NM_000863 | HTR1B | 4080 | 8127692 |
| NM_003512 | HIST1H2AC | 5322 | 8117372 |
| NM_001080113 | C14orf184 | | 7980859 |
| NR_024583 | POM121L8P | | 8071168 |
| NM_145232 | CTU1 | 4122 | 8038782 |
| AK123383 | LOC642648 | | 8076747 |
| NM_032479 | MRPL36 | | 8110861 |
| NM_007241 | SNF8 | 4144 | 8016508 |

In one embodiment, cycling hypoxia markers are selected from the list of the cycling hypoxia markers of Table 8 below, as well as their variants, fragments or equivalents.

TABLE 8

| GeneBank | Name of the | | |
|---|---|---|---|
| Accession Number | marker | Pathways | Probeset |
| NM_001168 | BIRC5 | 05200, 05210 | 8018860 |
| NM_032527 | ZGPAT | | 8064156 |
| NM_012322 | LSM5 | 03018, 03040 | 8138912 |
| NM_012394 | PFDN2 | | 7921786 |
| NM_002003 | FCN1 | | 8165011 |
| NM_001113201 | NACA | | 7964262 |
| NM_005608 | PTPRCAP | | 7949792 |
| NM_006858 | TMED1 | | 8034101 |
| NM_001551 | IGBP1 | | 8168087 |
| NM_001417 | EIF4B | 03013, 04150 | 7963575 |

In one embodiment of the invention, the signature of the invention comprises or consists of at least 2, preferably at least 3, more preferably at least 5, and even more preferably at least 10 cycling hypoxia markers.

In one embodiment of the invention, the signature of the invention comprises or consists of 2, 3, 4, 5, 6, 7, 8, 9 or 10 cycling hypoxia markers.

In one embodiment of the invention, the signature of the invention comprises at least 10 markers selected from the list of Table 1, preferably from the list of Table 2, more preferably from the list of Table 3, even more preferably from the list of Table 4, still even more preferably from the list of Table 5, still even more preferably from the list of Table 6, still even more preferably from the list of Table 7, and still even more preferably from the list of Table 8.

In one embodiment of the invention, the signature of the invention comprises or consists of 8, 9 or 10 markers selected from the list of Table 1, preferably from the list of Table 2, more preferably from the list of Table 3, even more preferably from the list of Table 4, still even more preferably from the list of Table 5, still even more preferably from the list of Table 6, still even more preferably from the list of Table 7, and still even more preferably from the list of Table 8.

In one embodiment of the invention, the signature of the invention comprises at least 3 markers. In one embodiment of the invention, the signature of the invention comprises one, two or three of BIRC5, IGBP1 and EIF4B. In one embodiment of the invention, the signature of the invention comprises at least the three markers BIRC5, IGBP1 and EIF4B. In one embodiment of the invention, the signature of the invention consists in the three markers BIRC5, IGBP1 and EIF4B.

In one embodiment, the signature of the invention comprises or consists of 1, 2 or 3 markers selected from the list of Table 8, preferably BIRC5, IGBP1 and/or EIF4B, and 5, 6, 7, 8, or 9 markers selected from the list of Table 1, preferably from the list of Table 2, more preferably from the list of Table 3, even more preferably from the list of Table 4, still even more preferably from the list of Table 5, still even more preferably from the list of Table 6, still even more preferably from the list of Table 7, and still even more preferably from the list of Table 8.

In one embodiment, the signature of the invention comprises or consists of 1 marker selected from the list of Table 8, and 1, 2, 3, 4, 5, 6, 7, 8, or 9 markers selected from the list of Table 5. In another embodiment, the signature of the invention comprises or consists of 2 markers selected from the list of Table 8, and 1, 2, 3, 4, 5, 6, 7, or 8 markers selected from the list of Table 5. In another embodiment, the signature of the invention comprises or consists of 3 markers selected from the list of Table 8, and 1, 2, 3, 4, 5, 6, or 7 markers selected from the list of Table 5. In another embodiment, the signature of the invention comprises or consists of 4 markers selected from the list of Table 8, and 1, 2, 3, 4, 5, or 6 markers selected from the list of Table 5. In another embodiment, the signature of the invention comprises or consists of 5 markers selected from the list of Table 8, and 1, 2, 3, 4, or 5 markers selected from the list of Table 5. In another embodiment, the signature of the invention comprises or consists of 6 markers selected from the list of Table 8, and 1, 2, 3, or 4 markers selected from the list of Table 5. In another embodiment, the signature of the invention comprises or consists of 7 markers selected from the list of Table 8, and 1, 2, or 3 markers selected from the list of Table 5. In another embodiment, the signature of the invention comprises or consists of 8 markers selected from the list of Table 8, and 1, or 2 markers selected from the list of Table 5. In another embodiment, the signature of the invention comprises or consists of 9 markers selected from the list of Table 8, and 1 marker selected from the list of Table 5.

In one embodiment, the signature of the invention comprises or consists of the 8 markers BIRC5, LMO2, NTHL1, RPS13, SNF8, LSM5, NACA and RPS28.

In another embodiment, the signature of the invention comprises or consists of the 9 markers BIRC5, C14orf156, LSM5, DYNLL1, SNF8, RPS28, RPS13, NACA and CHMP1B.

In another embodiment, the signature of the invention comprises or consists of the 9 markers BIRC5, EIF4B, C14orf156, LSM5, DYNLL1, SNF8, RPS28, RPS13 and NACA.

In a preferred embodiment, the signature of the invention comprises or consists of 10 markers selected from the list of Table 8, their variants, fragments and equivalents. More preferably, the signature comprises or consists of the 10 markers of Table 8, i.e. BIRC5, ZGPAT, LSM5, PFDN2, FCN1, NACA, PTPRCAP, TMED1, IGBP1 and EIF4B.

In one embodiment, the signature of the invention comprises or consists of the 9 markers LMO2, NTHL1, RPS13, SNF8, RPS28, MRPL17, TSG101, DYNLL1 and MKNK1.

In another embodiment, the signature of the invention comprises or consists of the 10 markers EIF4B, LMO2, NTHL1, RPS13, SNF8, RPS28, MRPL17, TSG101, DYNLL1 and MKNK1.

In another embodiment, the signature of the invention comprises or consists of the 10 markers BIRC5, EIF4B, LMO2, NTHL1, RPS13, SNF8, RPS28, MRPL17, TSG101 and DYNLL1.

In one embodiment, the signature of the invention does not consist of markers selected from the group consisting of PTPRCAP, HIST1H1C, C11orf10, HIST1H2AC, SSNA1, RPS28, RBX1, RPS13, MAD1L1, HIST1H4A and HIST1H4C.

The present invention also relates to a signature as hereinabove described, for the prognosis of cancer in a subject, wherein the signature of the invention is a signature of cycling hypoxia, i.e. comprises markers whose expression is different between a normoxic condition and a cycling hypoxia condition.

The present invention further relates to a non-invasive method for the prognosis of cancer in a subject, wherein said method comprises assessing the expression of markers in a sample of said subject, whose expressions are different between a normoxic condition and a cycling hypoxia condition. In one embodiment, the markers whose expressions are different between a normoxic condition and a cycling hypoxia condition together form a signature according to the invention.

In one embodiment of the invention, the method of the invention is for determining a personalized course of treatment of the subject. Indeed, according to the prognosis obtained, a personalized treatment may be administered to the subject.

In one embodiment of the invention, the expression of at least 2, preferably of at least 3, more preferably of at least 5, and even more preferably of at least 10 markers is assessed.

The present invention also relates to a signature as hereinabove described, wherein said signature is a predictive signature and is a signature of cycling hypoxia, i.e. comprises markers whose expression is different between a normoxic condition and a cycling hypoxia condition.

The present invention further relates to a non-invasive method for predicting or anticipating the response of a subject, preferably of a patient, to a specific treatment, wherein said method comprises assessing the expression of markers in a sample of said subject, whose expressions are different between a normoxic condition and a cycling hypoxia condition. In one embodiment, the markers whose expressions are different between a normoxic condition and a cycling hypoxia condition together form a predictive signature according to the invention.

In one embodiment of the invention, the method of the invention is for determining a personalized course of treatment of the subject. Indeed, according to the result obtained with the predictive signature, a personalized treatment may be administered to the subject.

In one embodiment of the invention, the expression of at least 2, preferably of at least 3, more preferably of at least 5, and even more preferably of at least 10 markers is assessed.

In one embodiment, the subject is diagnosed with cancer. In another embodiment, the subject is at risk of cancer. Examples of risks include, but are not limited to, familial history of cancer, genetic predisposition to cancer, environmental risks such as, for example, exposure to carcinogenic chemicals or other types of carcinogenic agents, diet, clinical factors such as, for example, hormonal deregulation or presence of another cancer-inducing disease, and the like.

In one embodiment, the subject is a cancer patient. In one embodiment, the subject is a patient with precancerous lesions or adenoma.

According to this embodiment, the signature or the non-invasive method may be for predicting overall survival of the subject, wherein the overall survival refers to the survival at 2 years, preferably at 3, 5, 8 years, more preferably at 10 years.

Still according to this embodiment, the signature or the non-invasive method may be for identifying patients who could benefit from a specific treatment, such as, for example, a chemotherapeutic treatment.

Still according to this embodiment, the signature or the non-invasive method may be for assessing the likelihood of a beneficial response of the patient to a specific anti-cancer treatment. The signature or the non-invasive method of the invention may also be for predicting the resistance of a patient to a specific anti-cancer treatment.

Still according to this embodiment, the signature or the non-invasive method of the invention may be for classifying a patient as a good prognosis or poor prognosis patient, wherein a good prognosis means that a patient is expected to have no distant metastases of a tumor within 2, preferably 3, 5, 8 or 10 years, and a poor prognosis means that a patient is expected to have distant metastases of a tumor within 2, preferably 3, 5, 8 or 10 years.

In another embodiment, signature or the non-invasive method of the invention may be for classifying a patient as a progression-free survival (PFS) patient, wherein progression-free survival means that the cancer does not get worse.

In a first embodiment, the subject previously received an anticancer treatment. In another embodiment, the subject did not receive any anticancer treatment. Examples of treatment include, but are not limited to, surgery for removing the tumor, chemotherapy and/or radiotherapy.

In one embodiment, the subject was previously treated for a cancer.

In one embodiment, the subject is considered as substantially healthy as regard to this cancer, i.e. the treatment is considered to have been successful.

According to this embodiment, the signature or the non-invasive method may be for assessing the likelihood of distal recurrence of the cancer. In one embodiment, distal recurrence refers to recurrence within 2 years, preferably within 3, 5, 8 years, more preferably within 10 years. In one embodiment, the term “recurrence” may refer to the reappearance of cancer (preferably of a tumor) either within the same organ or elsewhere in the body.

According to this embodiment, the signature or the non-invasive method may be for predicting overall survival of the subject, wherein the overall survival refers to the survival at 2 years, preferably at 3, 5, 8 years, more preferably at 10 years.

In one embodiment of the invention, the cancer is a neoplasm, i.e. a cancer characterized by the presence of at least one malignant tumor.

Examples of cancers include, but are not limited to, breast cancer, prostate cancer, lung cancer, colon cancer, cervix cancer, prostate cancer, brain cancer, liver cancer, kidney cancer and connective tissue cancer.

In one embodiment, the cancer may originate in the bladder, blood, bone, bone marrow, brain, breast, cervic area, colon, connective tissue, esophagus, eye and periocular tissues including subconjunctival tissues, duodenum, small intestine, large intestine, rectum, anus, gum, head, kidney, liver, lung, nasopharynx, neck, ovary, pancreas, prostate, skin, stomach, testis, tongue, or uterus.

Examples of cancer include, but are not limited to, fibrosarcoma, carcinoma, adenocarcinoma, lymphoma, blastoma, hepatoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, such as, for example, pancreatic carcinoma, glioblastoma, cervical cancer, ovarian cancer, liver cancer, bladder cancer, hepatoma, breast cancer, colon cancer, such as, for example, colon adenocarcinoma (including a colon adenocarcinoma grade II), colorectal cancer, such as, for example, colorectal carcinoma, endometrial or uterine carcinoma, salivary gland carcinoma, kidney or renal cancer, liver cancer, prostate cancer, such as, for example, prostate adenocarcinoma, vulval cancer, thyroid cancer, osteosarcoma, neuroblastoma, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; Burkitt's lymphoma; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

Other examples of cancers include, but are not limited to, adenocarcinoma, such as, for example, breast adenocarcinoma, prostate adenocarcinoma, liver adenocarcinoma or colorectal adenocarcinoma; ductal carcinoma, such as, for example, breast ductal carcinoma; carcinoma such as, for example, colorectal carcinoma, kidney carcinoma or squamous cell carcinoma (such as, for example, squamous cell carcinoma of the cervix); glioblastoma; hepatocellular carcinoma; hepatoma; or fibrosarcoma.

In one embodiment of the invention, the cancer is breast cancer, and the patient may be classified in different subgroups determined on the basis of clinicopathologic criteria. In one embodiment, the breast cancer patient is node negative or node positive. In another embodiment, the breast cancer patient is ER+ or ER−, wherein ER stands for estrogens receptor. In another embodiment, the breast cancer patient is HER2+ or HER2−, wherein HER2 stands for Human Epidermal Growth Factor Receptor-2. In one embodiment, the breast cancer patient is ER+/HER2−, ER−/HER2− or HER2+. In another embodiment, the breast cancer patient is ER+/HER2− node negative. In another embodiment, the breast cancer patient is ER+/HER2− node negative and did not receive any anticancer treatment.

In another embodiment of the invention, the cancer is colorectal cancer and the patient may be classified in different subgroups determined on the basis of clinicopathologic criteria, according to the American Joint Committee on Cancer (AJCC). In one embodiment, the colorectal cancer is a submucosa and muscularis propria tumor (stage I or 1). In another embodiment, the colorectal cancer is a tumor invading through the muscularis propria (stage II or 2). In another embodiment, the colorectal cancer is node positive (stage III or 3). In another embodiment, the colorectal cancer is associated with distant metastases (stage IV or 4).

In one embodiment of the invention, the non-invasive method of the invention for the prognosis of cancer in a subject comprises determining the expression profile of markers of a signature of the invention in a sample of said subject.

According to a preferred embodiment, the sample was previously taken from the subject, i.e. the method of the invention does not comprise a step of recovering a sample from the subject. Consequently, according to this embodiment, the method of the invention is a non-invasive method.

In one embodiment of the invention, the sample is a biopsy sample or a fine-needle aspirate. In one embodiment, the biopsy or the fine-needle aspiration is a biopsy or a fine-needle aspiration of the mass of cells suspected to be a tumor. In another embodiment, when a tumor has already been identified, the biopsy or the fine-needle aspiration is a biopsy or a fine-needle aspiration of this tumor.

In another embodiment of the invention, the sample is a sample of a bodily fluid. Examples of bodily fluids include, but are not limited to, blood, plasma, serum, lymph, ascetic fluid, cystic fluid, urine, bile, nipple exudate, synovial fluid, bronchoalveolar lavage fluid, sputum, amniotic fluid, peritoneal fluid, cerebrospinal fluid, pleural fluid, pericardial fluid, semen, saliva, sweat and alveolar macrophages.

In one embodiment of the invention, the non-invasive method of the invention comprises a step of comparing the expression profile of the markers of the signature of the invention measured in the sample of the subject with a reference expression profile, measured in a reference sample.

A reference expression profile can be relative to an expression profile derived from population studies, including without limitation, such subjects having similar age range, subjects in the same or similar ethnic group, similar cancer history and the like.

In one embodiment, the reference expression profile is constructed using algorithms and other methods of statistical and structural classification.

In one embodiment of the invention, the reference expression profile is derived from the measurement of the expression profile of markers of a signature of the invention in a control sample derived from one or more substantially healthy subjects. As used herein, a “substantially healthy subject” has not been previously diagnosed or identified as having or suffering from cancer.

In one embodiment of the invention, the reference expression profile is derived from the measurement of the expression profile of markers of a signature of the invention in a reference sample derived from a healthy tissue or sample of the same subject, whereas the expression profile to be compared was measured in a sample taken from a suspect mass of cells (i.e. from the suspected tumor) within the body of the subject.

In one embodiment of the invention, the reference expression profile is derived from the previous measurement of the expression profile of markers of a signature of the invention in a reference sample derived from the same subject, such as, for example, the expression profile measured one month before, preferably six months before, more preferably one year before or more.

In another embodiment of the invention, the reference expression profile is derived from the measurement of the expression profile of markers of a signature of the invention in a reference population. In one embodiment, the reference sample is thus derived from a reference population.

In one embodiment, the reference population comprises substantially healthy subjects, preferably at least 50, more preferably at least 100, more preferably at least 200 and even more preferably at least 500 substantially healthy subjects.

In another embodiment, the reference population comprises subjects diagnosed with cancer, preferably at least 100, more preferably at least 250, more preferably at least 500 subjects diagnosed with cancer.

In another embodiment of the invention, the reference expression profile is derived from the measurement of the expression profile in a reference sample derived from one or more subjects who are diagnosed or identified as having or suffering from cancer.

In one embodiment, the reference expression profile corresponds to the mean expression profile of the markers of the signature of the invention measured in the reference population.

In one embodiment of the invention, the reference expression profile corresponds to the median expression profile of the markers of the genetic signature of the invention measured in the reference population.

In one embodiment of the invention, the expression of the cycling hypoxia markers corresponds to the transcription level (i.e. expression of the RNA), or to the translation level (i.e. expression of the protein) of the marker.

In one embodiment of the invention, the expression of the cycling hypoxia markers is assessed at the protein level. Methods for determining a protein level in a sample are well-known in the art. Examples of such methods include, but are not limited to, immunohistochemistry, Multiplex methods (Luminex), western blot, enzyme-linked immunosorbent assay (ELISA), sandwich ELISA, fluorescent-linked immunosorbent assay (FLISA), enzyme immunoassay (EIA), radioimmunoassay (RIA) and the like.

In another embodiment of the invention, the expression of the cycling hypoxia markers is assessed at the RNA level. Methods for assessing the transcription level of a marker are well known in the prior art. Examples of such methods include, but are not limited to, RT-PCR, RT-qPCR, Northern Blot, hybridization techniques such as, for example, use of microarrays, and combination thereof including but not limited to, hybridization of amplicons obtained by RT-PCR, sequencing such as, for example, next-generation DNA sequencing (NGS) or RNA-seq (also known as “Whole Transcriptome Shotgun Sequencing”) and the like.

In one embodiment, the non-invasive method comprises the steps of:
extracting total RNA from the sample from the subject,
retro-transcribing these total RNA, thereby obtaining total cDNA,
specifically amplifying by PCR, preferably by qPCR, the cDNA corresponding to the cycling hypoxia markers of the signature of the invention, thereby determining the expression profile of the markers of the signature, and
comparing said expression profile with a reference expression profile determined in a reference sample.

In one embodiment, the expression profile of markers of the signature of the invention is measured using a polynucleotide microarray, so that the expression profiles of each of the markers of the signature of the invention are simultaneously measured.

In one embodiment, the non-invasive method comprises the steps of:
extracting total RNA from the sample from the subject, retro-transcribing these total RNA, thereby obtaining total cDNA from the sample, and labeling said total cDNA,
extracting total RNA from the reference sample, retro-transcribing these total RNA, thereby obtaining total cDNA from the reference sample, and labeling said total cDNA with a different label that the one used for the total cDNA of the sample from the subject,
applying the total cDNA from the sample from the subject, and the total cDNA from the reference sample, on a microarray, and
identifying markers which are differentially expressed between the sample from the subject and the reference sample, based on differential hybridization profile.

In one embodiment, the non-invasive method comprises the steps of:
in a first step, extracting total RNA from the reference sample, retro-transcribing these total RNA, thereby obtaining total cDNA from the reference sample, and labeling said total cDNA,
applying the total cDNA from the reference sample on a microarray, thereby obtaining a reference hybridization profile,
in a second, preferably subsequent step, extracting total RNA from the sample from the subject, retro-transcribing these total RNA, thereby obtaining total cDNA from the sample, and labeling said total cDNA,
applying the total cDNA from the sample from the subject on another microarray, thereby obtaining a sample hybridization profile, and
identifying markers which are differentially expressed between the sample from the subject and the reference sample, based on the differences of both hybridization profiles.

In one embodiment of the invention, the labeling of total cDNA is performed using fluorochromes, such as, for example, Cy3 and Cy5.

In one embodiment, the non-invasive method comprises the steps of:
extracting total RNA from the sample from the subject, retro-transcribing these total RNA, thereby obtaining total cDNA from the sample, and sequencing the total cDNA from the sample from the subject,
extracting total RNA from the reference sample, retro-transcribing these total RNA, thereby obtaining total cDNA from the reference sample, and sequencing the total cDNA from the reference sample, and
comparing the results of the cDNA sequencing and identifying markers which are differentially expressed between the sample from the subject and the reference sample.

In another embodiment, the non-invasive method comprises the steps of:
extracting total RNA from the sample from the subject, and sequencing the total RNA, preferably the total mRNA, from the sample from the subject,
extracting total RNA from the reference sample, and sequencing the total RNA, preferably the total mRNA from the reference sample, and
comparing the results of the RNA, preferably mRNA, sequencing and identifying markers which are differentially expressed between the sample from the subject and the reference sample.

In one embodiment of the invention, a marker of the invention is considered as differentially expressed in the sample from the subject as compared to a reference sample if both expression levels differ by a factor of at least 1.1, preferably at least 1.5, more preferably at least 2 and even more preferably at least 5.

In one embodiment of the invention, the post-translational modifications of a marker of the invention corresponds to a modification selected from the list comprising or consisting of phosphorylation, myristoylation, palmitoylation, isoprenylation, glypiation, lipoylation, O-, N- or S-acylation, alkylation, glycosylation, malonylation, hydroxylation, nucleotide addition, oxidation, sumoylation, ubiquitination, citrullination, deamidation, formation of disulfide bridges, proteolytic cleavage, racemization and the like.

Examples of methods for assessing post-translational modifications of a protein or peptide are well-known from the skilled artisan and include, but are not limited to, mass spectroscopy, methods using antibodies directed against the post-translational modification including, but not limited to, immunoblotting, immunoprecipitation, bead-based multiplexing, Eastern blotting, and the like.

The present invention also relates to a kit for measuring the expression profile of markers of the signature of the invention, and/or for implementing the non-invasive method of the invention. In one embodiment, the kit comprises means for determining the expression of the cycling hypoxia markers of the signature of the invention.

In one embodiment of the invention, the expression profile is measured at the protein level, and the kit of the invention comprises means for total protein extraction, as well as antibodies for detecting the cycling hypoxia markers of the invention.

The present invention also relates to a kit for determining the post-translational modification profile of markers of the signature of the invention, and/or for implementing the non-invasive method of the invention. In one embodiment, the kit comprises means for determining the post-translational modification of the cycling hypoxia markers of the genetic signature of the invention.

In another embodiment, the expression profile is measured at the RNA level, and the kit of the invention comprises means for total RNA extraction, means for reverse transcription of total RNA, and means for quantifying the expression of RNA corresponding to the cycling hypoxia markers of the invention.

In one embodiment, the means for determining the expression of the cycling hypoxia markers are PCR primers, preferably qPCR primers, specific for said cycling hypoxia markers. In one embodiment, said means for determining the expression of the cycling hypoxia markers are probes to detect qPCR amplicons obtained with qPCR primers as hereinabove described.

In one embodiment, said means for quantifying the expression of RNA corresponding to the cycling hypoxia markers of the invention is PCR, preferably qPCR.

Examples of set of primers and probes that may be used for quantifying the expression of the cycling hypoxia markers of Table 8 are shown in the Table 9 below:

The TaqMan gene expression assay references can be found on http://www.invitrogen.com/site/us/en/home/Products-and-Services/Applications/PCR/real-time-per/real-time-per-assays/taqman-gene-expression/single-tube-taqman-gene-expression-analysis.html.

TABLE 9

| | | TaqMan gene |
|---|---|---|
| | | expression assay |
| GeneBank | Name of the | references |
| Accession Number | marker | (primer/probe set) |
| NM_001168 | BIRC5 | Hs04194392_s1 |
| NM_032527 | ZGPAT | Hs00738790_m1 |
| NM_012322 | LSM5 | Hs01123609_g1 |
| NM_012394 | PFDN2 | Hs00276171_m1 |
| NM_002003 | FCN1 | Hs00157572_m1 |
| NM_001113201 | NACA | Hs01903640_uH |
| NM_005608 | PTPRCAP | Hs02519237_s1 or |
| | | Hs00174778_m1 |
| NM_006858 | TMED1 | Hs00183648_m1 or |
| | | Hs00970159_g1 |
| NM_001551 | IGBP1 | Hs00426831_mH |
| NM_001417 | EIF4B | Hs01903212_gH or |
| | | Hs00251278_s1 |

In one embodiment of the invention, set of primers and probe that are used for quantifying the expression of the cycling hypoxia marker BIRC5 are the following sequences: AGGGCTGAAGTCTGGCGTAA (forward primer, SEQ ID NO:1), AACAATCCACCCTGCAGCTCTA (reverse primer, SEQ ID NO:2) and ATGATGGATTTGATTCGC (probe, SEQ ID NO:3).

In one embodiment of the invention, set of primers and probe that are used for quantifying the expression of the cycling hypoxia marker NACA are the following sequences: CCACCCCTAAATCTGCTGGAA (forward primer, SEQ ID NO:4), TCCAGACCCCTTGTTGTTCTTC (reverse primer, SEQ ID NO:5) and CCCTGTCCCAACCC (probe, SEQ ID NO:6).

In one embodiment of the invention, set of primers and probe that are used for quantifying the expression of the cycling hypoxia marker IGBP1 are the following sequences: GTCCGCGCTCGCCTAAT (forward primer, SEQ ID NO:7), GAGAGAGGAACCCGGAAGATCT (reverse primer, SEQ ID NO:8) and CTTTATCAAGGTTGCCTTTG (probe, SEQ ID NO:9).

In one embodiment of the invention, the kit of the invention also comprises primers for amplifying reference genes. Reference genes are genes expressed at a constant level among different tissues and/or conditions. Examples of reference genes include, but are not limited to, β-actin, genes encoding ribosomal proteins and the like.

In one embodiment of the invention, the kit of the invention comprises means for total RNA extraction, means for reverse transcription of total RNA, and reagents for carrying out a quantitative PCR as hereinabove described (such as, for example, primers, buffers, enzyme, and the like). In one embodiment, the kit of the invention also comprises a reference sample.

In one embodiment of the invention, the kit of the invention comprises DNA probes, which may be hybridized to the qPCR amplicons to detect said cycling hypoxia marker.

In one embodiment, the means for determining the expression of the markers of the signature is a microarray comprising probes specific for said cycling hypoxia markers.

In one embodiment, said means for quantifying the expression of RNA corresponding to the cycling hypoxia markers of the invention is a microarray. The present invention thus also relates to microarrays for measuring the RNA expression profile of markers of the signature of the invention, and/or for implementing the non-invasive method of the invention.

In one embodiment of the invention, the microarray of the invention comprises DNA probes, which may be hybridized to the retro-transcribed RNA corresponding to the cycling hypoxia markers of the invention.

In one embodiment of the invention, the microarray of the invention comprises probes specific of at least 3, 5, 10, 15, 25, 30, 40, 50, 60, 70, 80, 90, 100, 200, 300, 400, 500, 750, 1000, or at least 1350 cycling hypoxia markers of the invention, and up to the 1379 cycling hypoxia markers of Table 1.

In one embodiment of the invention, the microarray of the invention comprises probes specific of the 1379 markers of Table 1, and/or of the 651 markers of Table 2, and/or of the 298 markers of Table 3, and/or of the 167 markers of Table 4, and/or of the 96 markers of Table 5, and/or of the 74 markers of Table 6, and/or of the 37 markers of Table 7, and/or of the 10 markers of Table 8.

Examples of probes specific of the cycling hypoxia markers of the invention include, but are not limited to those corresponding to the probesets shown in the columns “probeset” of Tables 1 to 8, wherein numbers correspond to Affymetrix references. The oligonucleotide sequence corresponding to the Affymetrix references may be easily found on the product support page of Affymetrix (https://www.affymetrix.com/user/login.jsp?toURL=/analysis/netaffx/xmlquery ex.affx ?netaffx=wtgene_transcript) by selecting Human Gene 1.X ST.

In one embodiment of the invention, the microarray comprises probes specific of the 96 markers of Table 5.

In one embodiment of the invention, the microarray comprises probes specific of the 10 markers of Table 8.

In one embodiment, the microarray of the invention also comprises probes for reference genes. Reference genes are genes expressed at a constant level among different tissues and/or conditions. Examples of reference genes include, but are not limited to, β-actin, genes encoding ribosomal proteins and the like.

In one embodiment, the microarray of the invention also comprises probes for quality control genes. Quality control genes expression allows verifying the quality of the microarray and/or of the cDNA applied on the microarray.

In one embodiment of the invention, the kit of the invention comprises means for total RNA extraction, means for reverse transcription of total RNA, and a microarray of the invention as well as buffers and materials for use thereof. In one embodiment, the kit of the invention also comprises a reference sample.

In one embodiment, the means for determining the expression of the markers of the signature is sequencing means, allowing sequencing total RNA, preferably mRNA, or total cDNA of the sample from the subject, preferably using high-throughput sequencing technologies, more preferably using the RNA-Seq technology.

Examples of means for total sequencing of cDNA of a sample include, but are not limited to, poly(T) oligos, poly(T) magnetic beads, probes for removing ribosomal RNA, reverse transcriptase, emulsion PCR buffers and reagents, bridge amplification buffers and reagents, ligase and the like.

In another embodiment of the invention, the non-invasive method of the invention also comprises a step of measuring clinical data. Examples of clinical data which may be relevant for the prognosis of cancer in a subject and/or for predicting the response of a subject, preferably of a patient, to a specific treatment include, but are not limited to, gender, age, size of the tumor, tumor histological grade, lymph node status, presence of a treatment, presence of metastases, specific expression profiles (such as, for example, expression status for estrogen receptor or for HER2 receptor), Nottingham grading system (NGS), Nottingham Prognostic Index (NPI), and the like.

In one embodiment of the invention, the non-invasive method of the invention comprises a step of combining the expression profiles of the markers of the signature of the invention and optionally of the value of clinical data as hereinabove described in a score.

In one embodiment, said combination is a mathematical combination in a mathematical function. Preferably, said mathematical function is a weighted sum. In one embodiment, the weighted sum is adjusted on the reference sample.

In one embodiment, the method of the invention comprises comparing the score obtained with a threshold value. In one embodiment, the threshold value corresponds to the score obtained in a reference population or in a reference sample. In another embodiment, the weighted sum is adjusted on the reference sample such that the threshold value is equal to 0.

In one embodiment, the score of the invention is a prognostic score, and may be used for the prognosis of cancer in the subject. In another embodiment, the score of the invention is a predictive score, and may be used for predicting the response of a subject, preferably of a patient, to a specific treatment.

The present invention thus also relates to a non-invasive method for the prognosis of cancer in a subject, or for predicting the response of a subject to a specific treatment, wherein said method comprises:
assessing the expression profiles of markers of a signature comprising at least 2 cycling hypoxia markers in a sample from said subject, and
mathematically combining the measured expression profiles in a score.

The present invention presents the following advantages:
(i) As cycling hypoxia is a hallmark of a vast majority of tumors, the signature of the invention allows the prognosis of all tumor types, and of all neoplasms;
(ii) The prognosis method of the invention, based on the identification of the signature of the invention, is easy and rapid to implement, as the inventors showed that a signature of the invention, comprising as few as about 10 markers, allows an efficient prognosis.

### BRIEF DESCRIPTION OF THE DRAWINGS

FIG. 1 is a heatmap depicting transcripts from a signature of the invention (CycHyp) either underexpressed (green) or overexpressed (red) (centered to median values). Each column corresponds to a specific Human Gene 1.0 ST probeset; each line represents a specific cell line either maintained under normoxia (black label) or exposed to cycling hypoxia (red label); cell under normoxia and cycling hypoxia are perfectly separated in two distinct clusters, except for one cycling hypoxia sample in the normoxia cluster.

FIG. 2 is a combination of Kaplan-Meier survival curves of patients with primary breast cancer, as determined by using a signature of the invention (CycHyp). (A): All patients; (B): ER+/HER2− patients; (C): node-negative ER+/HER−2 patients; and (D): node-negative, untreated patients (DFS Mantel-Cox comparison).

FIG. 3 is a comparison of the prognostic potential of the CycHyp signature vs. Gene 70 (Mammaprint), Gene 76 and Oncotype Dx signatures to discriminate patients with progressing disease versus disease-free at 5-years. (A) Balance Classification Rate (BCR), i.e. the arithmetic average between specificity and sensitivity (also depicted) determined on the validation sets only (to avoid an optimistic bias if computed on the training set) [see text for p-values] and Concordance Index (CI) between high and low risk patients, [p<0.05 vs. Oncotype DX, p=0.07 vs. Gene70 and p=0.063 vs. Gene76]. (B.) Kaplan-Meier survival curves of node-negative, untreated ER+/HER2− patients, as determined by using the indicated signature (DFS Mantel-Cox comparison); hazard ratio (HR) for the prediction in high risk vs. low risk groups are presented with their associated confidence interval and p-values.

FIG. 4 is a combination of Kaplan-Meier survival curves of node-negative, untreated ER+/HER2− patients stratified by using a signature of the invention (CycHyp) to detect (A) false-positive patients among those identified at high risk based on the NPI nomenclature and (B) false-negative patients among those identified at low risk based on the NPI nomenclature (DFS Mantel-Cox comparison).

FIG. 5 is a combination of Kaplan-Meier survival curves of patients with primary breast cancer stratified at low or high risk according to a signature of the invention (CycHyp) or the NPI nomenclature (DFS Mantel-Cox comparison). (A): all patients; (B): ER+/HER2− patients; (C): node-negative ER+/HER2− patients; (D): node-negative, untreated ER+/HER2− patients.

FIG. 6 is a combination of Kaplan-Meier survival curves of patients with primary breast cancer stratified at low or high risk according to a 10-probesets signature (see Table 14) of the invention.

FIG. 7 is a combination of Kaplan-Meier survival curves of patients with primary breast cancer stratified at low or high risk according to a 10-probesets signature of the invention. This is an example of a 10-probesets signature selected from those shown in Table 11 without any overlap with the probes reported in Table 14. Note that the probesets for BIRC5 and NACA are different in Table 14 and Table 15.

FIG. 8 is a combination of Kaplan-Meier survival curves of patients with primary breast cancer stratified at low or high risk according to a 10-probesets signature of the invention. This is an example of a 10-probesets signature selected from those shown in Table 11 with a single one (BIRC5) overlapping with those reported in Table 14. Note that the probesets for NACA are different in Table 14 and Table 16.

FIG. 9 is a combination of Kaplan-Meier survival curves of patients with primary breast cancer stratified at low or high risk according to a 10-probesets signature of the invention. This is an example of a 10-probesets signature selected from those shown in Table 11 with two probesets (BIRC5 and EIF4B) overlapping with those reported in Table 14. Note that the probesets for NACA are different in Table 14 and Table 17.

FIG. 10 is a graph representing the power of discrimination in high vs. low risk groups (expressed as the logarithm of the p-values of the log rank) of the ContHyp (left) and CycHyp (right) signatures (see black dots) versus 1,000 randomly generated signatures of breast cancer patients (gray shapes depicting their distribution).

FIG. 11 is a combination of Kaplan-Meier survival curves of patients with primary breast cancer stratified at low or high risk according to a 10-probesets signature of the invention. This is an example of a 10-probesets signature (see Table 18′) selected from those shown in Table 11 without any overlap with the probes reported in Table 14.

FIG. 12 is a combination of Kaplan-Meier survival curves of patients with primary breast cancer stratified at low or high risk according to a 10-probesets signature of the invention. This is an example of a 10-probesets signature selected from those shown in Table 18 with a single one (EIF4B) overlapping with those reported in Table 14.

FIG. 13 is a combination of Kaplan-Meier survival curves of patients with primary breast cancer stratified at low or high risk according to a 10-probesets signature of the invention. This is an example of a 10-probesets signature selected from those shown in Table 18 with two probesets (EIF4B and BIRC5) overlapping with those reported in Table 14.

FIG. 14 is combination of Kaplan-Meier survival curves of node-negative, untreated ER+/HER2− patients stratified at low or high risk according to the ContHyp signature (DFS Mantel-Cox comparison).

FIG. 15 is a graph representing the power of discrimination in high vs. low risk groups (expressed as the logarithm of the p-values of the log rank) of the CycHyp signatures (see black dots) versus 1,000 randomly generated signatures of colorectal cancer patients (gray shapes depicting their distribution).

FIG. 16 is a combination of Kaplan-Meier survival curves of stage II colorectal cancer patients stratified at low or high risk according to the CycHyp signature.

FIG. 17 is a combination of Kaplan-Meier survival curve of patients with primary breast cancer stratified at low or high risk according to a 3-probesets signature (see Table 21) of the invention.

### EXAMPLES

The present invention is further illustrated by the following examples.

### Example 1

### CycHyp Signature on Breast Cancer Patients

### Patients and Methods

### Tumor Cells

Twenty tumor cells (see Table 10 for details) were submitted to cycling hypoxia (CycHyp), i.e. 24 cycles of 30 min incubation under normoxia and 30 min incubation under hypoxic (1% O₂) conditions to reproduce the frequency of tumor hypoxic fluctuations, as previously reported (Dewhirst, Radiat Res 172:653-665, 2009).

TABLE 10
List of Human Tumor Cells used for Microarray Analysis.

| Cell line | Organ | Disease |
|---|---|---|
| MCF-7 | Breast | Adenocarcinoma |
| MDA-MB-231 | Breast | Adenocarcinoma |
| T47D | Breast | Ductal carcinoma |
| A549 | Lung | Carcinoma |
| Widr | Colon | Colorectal adenocarcinoma |
| HCT116^{WTP53} | Colon | Colorectal carcinoma |
| HCT116^{-/}^{_{—}}^{P53} | Colon | Colorectal carcinoma |
| HT29 | Colon | Colorectal adenocarcinoma |
| Colo-205 | Colon | Colorectal adenocarcinoma |
| LoVo | Colon | Colorectal adenocarcinoma |
| HCT15 | Colon | Colorectal adenocarcinoma |
| SiHa | Cervix | Squamous cell carcinoma |
| PC3 | Prostate | Adenocarcinoma |
| U373 | Brain | Glioblastoma |
| HepG2 | Liver | Hepatocellular carcinoma |
| Hep3B | Liver | Hepatocellular carcinoma |
| PLC/PRF/5 | Liver | Hepatoma |
| SK-HEP-1 | Liver | Adenocarcinoma |
| A498 | Kidney | Carcinoma |
| HT1080 | Connective tissue | Fibrosarcoma |

### Identification of the Signature

mRNA extracts from each tumor cell cultured under both the above conditions (normoxia and cycling hypoxia) were analysed by hybridization on Human Gene 1.0 ST Affymetrix microarrays (GEO access number: GSE42416). The extent of the resulting tumor cell datasets (20 samples in each of the three conditions) led us to resort on a resampling mechanism to increase the robustness of the signatures to be identified. For every resampling experiment, a subset of 90% of the samples was chosen uniformly at random without replacement. Differentially expressed probesets were assessed on each subset according to a t-test and the corresponding p-values were reported. The 100 probesets with the lowest p-values, averaged over 200 resamplings, formed the CycHyp signature. All such expression differences were highly significant (p<10⁻⁴) after Benjamini-Hochberg FDR correction for the multiplicity of the test (Benjamini et al, J R Stat Soc 57:289-300, 1995). The 100 HGU1.0 ST probesets forming the CycHyp signature corresponded to 94 unique Entrez GeneID in the NCBI database, out of which 69 genes were available on the HGU133a platform (i.e., the technology used in most clinical studies considered here). Those 69 genes were represented by 87 HGU133a probesets. The few datasets collected on HGU133plus2 were reduced to the probesets also present on HGU133a, thus with an identical CycHyp signature of 87 probesets.

### Patient Data Sets

All breast cancer expression data were summarized with MASS and represented in log 2 scale (except for GSE6532 already summarized with RMA). Breast cancer subtypes (ER+/HER2−, ER−/HER2− and HER2+) were identified with the genefu R package (Haibe-Kains et al, Genome Biol 11:R18, 2010). Disease-free survival at 5 years was used as the survival endpoint. The data from all patients were censored at 10 years to have comparable follow-up times across clinical studies (Haibe-Kains et al, Bioinformatics 24:2200-2208, 2008).

### Prognostic Models of the Clinical Outcome

The VDX dataset (GSE2034 and GSE5327 from the GEO database) was considered as a reference because of its large number of node-negative untreated patients (Wang et al, Lancet 365:671-679, 2005). This dataset formed the training set used to estimate a prognostic model of the clinical outcome. A risk score for each patient was computed from a penalized Cox proportional hazards model implemented in the Penalized R package (Goeman, Biom J 52:70-84, 2010). Prediction into a high risk vs. low risk group resulted from a predefined threshold value on this risk score. The decision threshold was chosen on the training set to maximize the specificity and sensitivity of the discrimination between patients with progressing disease versus disease-free patients at 5 years. Following the methodology described by Haibe-Kains et al. (Haibe-Kains et al, Bioinformatics 24:2200-2208, 2008), all other datasets were used as validations to assess the prognostic performances on independent samples. Performance metrics included the balanced classification rate (BCR), i.e. the arithmetic average between specificity and sensitivity (determined on the validation sets only to avoid an optimistic bias if computed on the training set), the concordance index (CI) (Harrell et al, Stat Med 15:361-387, 1996) and the hazard ratio (HR) (Cox, J R Stat Soc 34:187-220, 1972) for the prediction in high risk vs. low risk groups, with their associated confidence interval and p-values. Prognostic performances of a penalized Cox model defined on the CycHyp signature were also compared with well-established prognosis models for breast cancer, namely Gene 70 (Mammaprint) (van′t Veer et al, Nature 415:530-536, 2002), Gene 76 (Wang et al, Lancet 365:671-679, 2005) and Oncotype DX (Paik et al, N Engl J Med 351:2817-2826, 2004) signatures. Those existing signatures were associated to specific prognostic models implemented in the genefu R package (Haibe-Kains et al, Genome Biol 11:R18, 2010).

### Results

### Identification of the CycHyp Signature

Tumor cells were submitted to cycling hypoxia for 24 hours or maintained under normoxic conditions for the same period of time. Corresponding mRNA samples were analysed by hybridization using Human Gene 1.0 ST Affymetrix microarrays. Gene expression profiles of each cell type under normoxia vs. cycling hypoxia were produced to identify the most differentially expressed probesets.

The CycHyp signature was determined as the top 100 probesets with the lowest average pvalues over 200 resamplings, corresponding to 96 markers. These probesets are shown in the Table 11 below.

TABLE 11

| | | GenBank | Name of the |
|---|---|---|---|
| | Probeset | Accession Number | marker |
| 1 | 8018860 | NM_001168 | BIRC5 |
| 2 | 8064156 | NM_032527 | ZGPAT |
| 3 | 8138912 | NM_012322 | LSM5 |
| 4 | 7921786 | NM_012394 | PFDN2 |
| 5 | 8165011 | NM_002003 | FCN1 |
| 6 | 7964262 | NM_001113201 | NACA |
| 7 | 7949792 | NM_005608 | PTPRCAP |
| 8 | 8034101 | NM_006858 | TMED1 |
| 9 | 8168087 | NM_001551 | IGBP1 |
| 10 | 7963575 | NM_001417 | EIF4B |
| 11 | 8124397 | NM_005319 | HIST1H1C |
| 12 | 7975989 | NM_031210 | SLIRP |
| 13 | 8127692 | NM_000863 | HTR1B |
| 14 | 8127087 | NM_000847 | GSTA3 |
| 15 | 7941122 | NM_013299 | SAC3D1 |
| 16 | 7998692 | NM_002528 | NTHL1 |
| 17 | 8073623 | NM_001044370 | MPPED1 |
| 18 | 8014865 | NM_006160 | NEUROD2 |
| 19 | 8005726 | NM_021012 | KCNJ12 |
| 20 | 7966631 | NM_022363 | LHX5 |
| 21 | 8037853 | NM_017854 | TMEM160 |
| 22 | 8104136 | NM_018942 | HMX1 |
| 23 | 7948606 | NM_014206 | C11orf10 |
| 24 | 8044773 | NM_006770 | MARCO |
| 25 | 7947015 | NM_006292 | TSG101 |
| 26 | 7931553 | NM_003577 | UTF1 |
| 27 | 7956876 | NM_032338 | LLPH |
| 28 | 8117372 | NM_003512 | HIST1H2AC |
| 29 | 8001329 | NM_004352 | CBLN1 |
| 30 | 8027205 | NM_015965 | NDUFA13 |
| 31 | 8042896 | NM_016170 | TLX2 |
| 32 | 7911532 | NM_017900 | AURKAIP1 |
| 33 | 8039923 | NM_017900 | AURKAIP1 |
| 34 | 7992043 | BC001181 | FAM173A |
| 35 | 8063074 | NM_080603 | ZSWIM1 |
| 36 | 7992191 | NM_012217 | TPSD1 |
| 37 | 8108435 | NM_181838 | UBE2D2 |
| 38 | 8165309 | NM_003792 | EDF1 |
| 39 | 7946267 | NM_022061 | MRPL17 |
| 40 | 7945536 | NM_016564 | CEND1 |
| 41 | 8159609 | NM_003731 | SSNA1 |
| 42 | 8005471 | NM_001031 | RPS28 |
| 43 | 8025395 | NM_001031 | RPS28 |
| 44 | 7942824 | NM_001031 | RPS28 |
| 45 | 8170753 | NM_014370 | SRPK3 |
| 46 | 8032718 | NM_001348 | DAPK3 |
| 47 | 7967067 | NM_001037495 | DYNLL1 |
| 48 | 8159654 | NM_015456 | COBRA1 |
| 49 | 8011212 | NM_003001 | SDHC |
| 50 | 8011968 | NM_016060 | MED31 |
| 51 | 7977440 | NR_026800 | KIAA0125 |
| 52 | 8016508 | NM_007241 | SNF8 |
| 53 | 8168567 | NM_000307 | POU3F4 |
| 54 | 8086317 | NM_031899 | GORASP1 |
| 55 | 8052834 | BC005079 | C2orf42 |
| 56 | 8073334 | NM_014248 | RBX1 |
| 57 | 7915846 | NM_003684 | MKNK1 |
| 58 | 8071920 | NM_004175 | SNRPD3 |
| 59 | 8032371 | NM_031213 | FAM108A1 |
| 60 | 7924884 | NM_003493 | HIST3H3 |
| 61 | 8006845 | NM_000981 | RPL19 |
| 62 | 7946812 | NM_001017 | RPS13 |
| 63 | 7949015 | NM_001144936 | C11orf95 |
| 64 | 8009784 | NM_015971 | MRPS7 |
| 65 | 8174509 | NM_005274 | GNG5 |
| 66 | 7906235 | NM_005973 | PRCC |
| 67 | 8020179 | NM_020412 | CHMP1B |
| 68 | 7947450 | NM_005574 | LMO2 |
| 69 | 8064370 | NM_004609 | TCF15 |
| 70 | 7955896 | NM_016057 | COPZ1 |
| 71 | 8137805 | NM_003550 | MAD1L1 |
| 72 | 8117334 | NM_003538 | HIST1H4A |
| 73 | 8117368 | NM_003542 | HIST1H4C |
| 74 | 7977507 | NR_002312 | RPPH1 |
| 75 | 7949410 | BC018448 | MALAT1 |
| 76 | 8150433 | NM_152568 | NKX6-3 |
| 77 | 8071168 | NR_024583 | POM121L8P |
| 78 | 7989611 | NM_032231 | FAM96A |
| 79 | 7980859 | NM_001080113 | |
| 80 | 8032782 | NM_144615 | TMIGD2 |
| 81 | 8110861 | NM_032479 | MRPL36 |
| 82 | 7901687 | NM_182532 | TMEM61 |
| 83 | 7916130 | NM_138417 | KTI12 |
| 84 | 8048712 | BC033986 | LOC440934 |
| 85 | 8018993 | NM_001082575 | RBFOX3 |
| 86 | 8032601 | NM_032753 | RAX2 |
| 87 | 8010719 | NM_144999 | LRRC45 |
| 88 | 8036584 | NM_002307 | LGALS7 |
| 89 | 8133209 | NR_003666 | SPDYE7P |
| 90 | 8159501 | NM_178536 | LCN12 |
| 91 | 8028546 | NM_002307 | LGALS7 |
| 92 | 8065013 | ENST00000427835 | |
| 93 | 8018502 | NM_173547 | TRIM65 |
| 94 | 7903294 | NM_033055 | HIAT1 |
| 95 | 7989473 | NM_001007595 | C2CD4B |
| 96 | 8054449 | AK095987 | FLJ38668 |
| 97 | 8081867 | NM_016589 | TIMMDC1 |
| 98 | 7934544 | NM_144589 | COMTD1 |
| 99 | 7968260 | NM_145657 | GSX1 |
| 100 | 8022952 | NM_020180 | CELF4 |

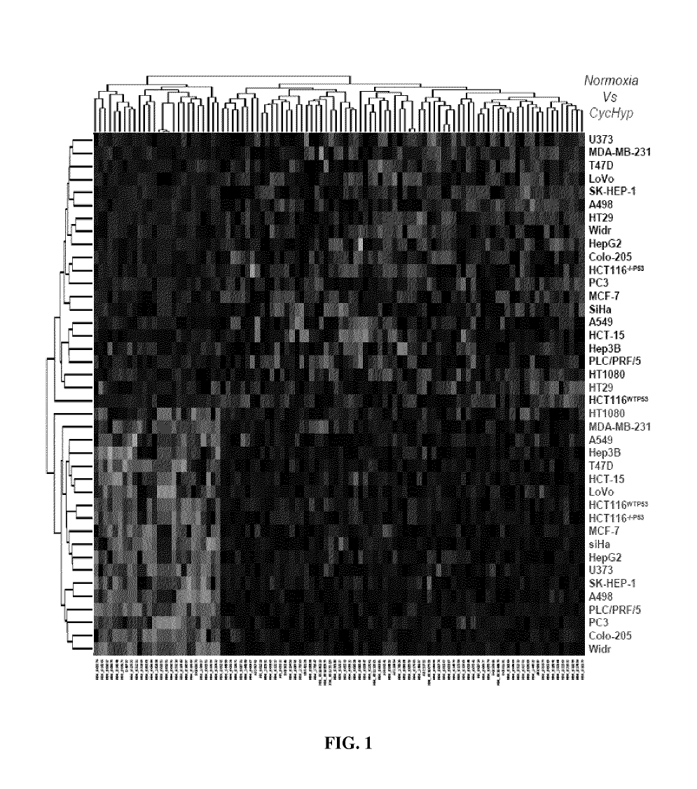

The heatmap (FIG. 1) made with the 100 probe sets of the CycHyp signature confirmed its excellent potential of discrimination between cycling hypoxia and normoxia.

### The CycHyp Signature Predicts Clinical Outcome in Breast Cancer Patients

To evaluate the prognostic value of the CycHyp signature, we focused on breast cancer because of the very large amounts of well-annotated clinical data sets available and a clearly identified need to discriminate between patients at low and high risks among subgroups determined on the basis of clinicopathologic criteria (Reis-Filho et al, Lancet 378:1812-1823, 2011; Prat et al, Nat Rev Clin Oncol 9:48-57, 2011). Publicly available GEO data sets allowed us to collect information on the survival of 2,150 patients with primary breast cancer (see clinical features in Table 12).

TABLE 12
Breast Cancer Patient Demographics and Characteristics

| | | | | | | | ER+/HER2− | |
|---|---|---|---|---|---|---|---|---|
| | | | | | ER+/HER2− | | Node neg. | |
| | All patients | | ER+/HER2− | | Node neg. | | Untreated | |
| | n = 2150 | | n = 1452 | | n = 899 | | n = 590 | |
| | No | % | No | % | No | % | No | % |
| Age | | | | | | | | |
| ≦50 | 649 | 30 | 388 | 27 | 218 | 24 | 190 | 32 |
| >50 | 945 | 44 | 649 | 45 | 367 | 41 | 237 | 40 |
| NA | 556 | 26 | 415 | 28 | 314 | 35 | 163 | 28 |
| Tumor size | | | | | | | | |
| ≦2 cm | 742 | 35 | 537 | 37 | 474 | 53 | 424 | 72 |
| >2 cm | 473 | 22 | 326 | 22 | 210 | 23 | 158 | 28 |
| NA | 935 | 43 | 589 | 41 | 215 | 24 | 8 | 1 |
| Grade | | | | | | | | |
| 0-1 | 224 | 10 | 200 | 14 | 148 | 17 | 104 | 18 |
| 2 | 605 | 28 | 485 | 33 | 346 | 38 | 270 | 46 |
| 3 | 487 | 23 | 206 | 14 | 162 | 18 | 137 | 23 |
| NA | 834 | 39 | 561 | 39 | 243 | 27 | 79 | 13 |
| Node status | | | | | | | | |
| Negative | 1329 | 62 | 899 | 62 | 899 | 100 | 590 | 100 |
| Positive | 821 | 38 | 553 | 38 | 0 | 0 | 0 | 0 |
| Estrogen | | | | | | | | |
| receptor | | | | | | | | |
| Negative | 443 | 21 | 0 | 0 | 0 | 0 | 0 | 0 |
| Positive | 1607 | 75 | 1452 | 100 | 899 | 100 | 590 | 100 |
| NA | 100 | 4 | 0 | 0 | 0 | 0 | 0 | 0 |
| HER2 status | | | | | | | | |
| Negative | 1835 | 85 | 1452 | 100 | 899 | 100 | 590 | 100 |
| Positive | 315 | 15 | 0 | 0 | 0 | 0 | 0 | 0 |
| Treatment | | | | | | | | |
| None | 901 | 42 | 590 | 41 | 590 | 66 | 590 | 100 |
| Chemo- | 691 | 32 | 410 | 28 | 73 | 8 | 0 | 0 |
| therapy | | | | | | | | |
| Hormono- | 558 | 26 | 452 | 31 | 236 | 26 | 0 | 0 |
| therapy | | | | | | | | |

In order to exploit these data sets, we first transferred the Gene 1.0ST technology in the HGU133 platform. The 100 HGU1.0 ST probesets forming the CycHyp signature correspond to 94 unique Entrez GeneID in the NCBI database (Table 11), out of which 69 genes were available on the HGU133a platform. Those 69 genes are represented by 87 HGU133a probesets. The few datasets collected on HGU133plus2 were reduced to the probesets also present on HGU133a.

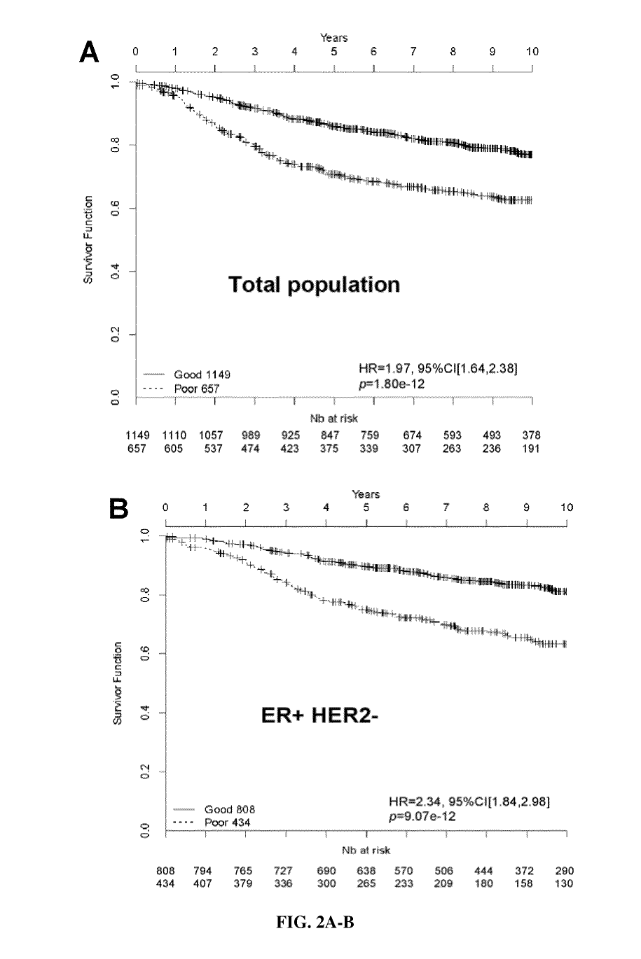

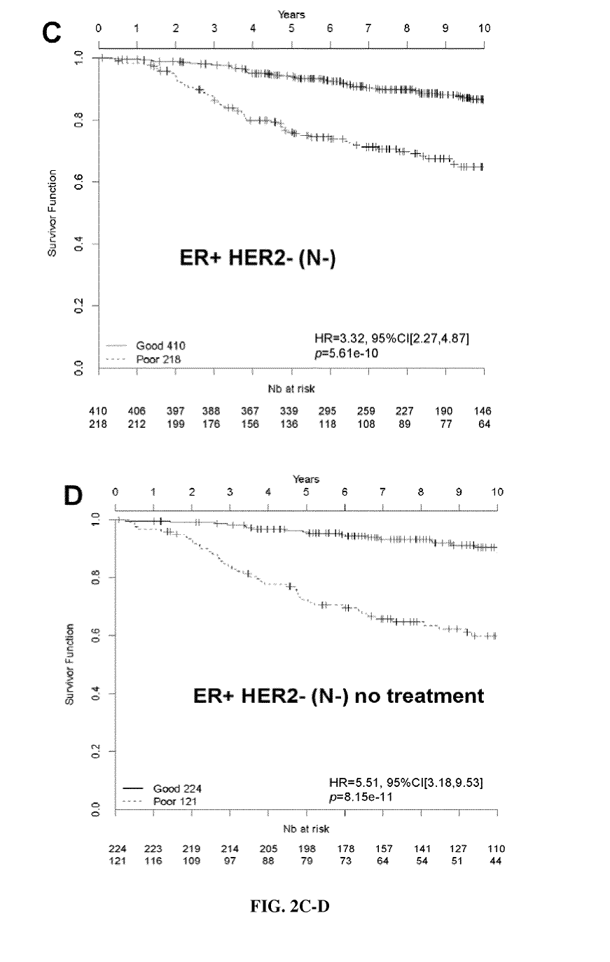

We then used the VDX dataset (GSE2034 and GSE5327) as a reference because of its large number of node negative untreated patients (Wang et al, Lancet 365:671-679, 2005). This training dataset was used to estimate a prognostic Cox proportional hazard model built on the CycHyp signature. The other datasets were used according to the methodology described by Haibe-Kains and colleagues (Haibe-Kains et al, Bioinformatics 24:2200-2208, 2008), to assess the prognostic performance of the CycHyp signature on independent samples. We first chose to evaluate our signature independently of the receptor status of the tumors. The prognostic potential of the CycHyp signature to discriminate between patients at low or high risk was confirmed with a HR=1.97 and a p-value=1.8. 10⁻¹² (FIG. 2A). We then focused on the ER+HER2− population which is known to be heterogeneous and thus difficult to treat (Reis-Filho et al, Lancet 378:1812-1823, 2011; Prat et al, Nat Rev Clin Oncol 9:48-57, 2011). The discriminating capacity of the CycHyp signature remained strikingly high in the ER+HER2− patient populations (HR=2.34, p-value=9. 10⁻¹², FIG. 2B). Finally, among this subpopulation of patients, we considered those with a node negative status (FIG. 2C) and among the latter, those who did not receive any treatment (FIG. 2D). Hazard ratios rose to 3.32 and 5.51 in these conditions (p-values=5.61. 10⁻¹⁰ and 8.15. 10⁻¹¹, respectively), further supporting the discriminating potential of the CycHyp signature. In particular, the data presented in FIG. 2D allowed to exclude any confounding influence of the potential benefit arising from the treatment administered to these patients and thus clearly identified a population of patients who remained inadequately untreated.

### The CycHyp Signature Provides Significant Additional Prognostic Information to Available Multigene Assays

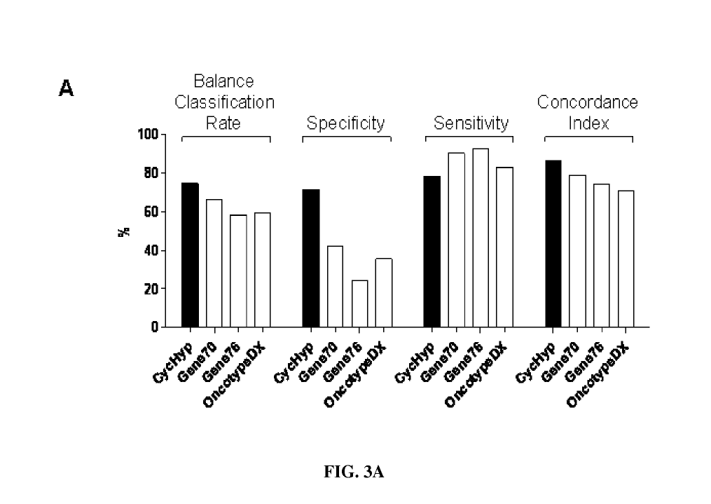

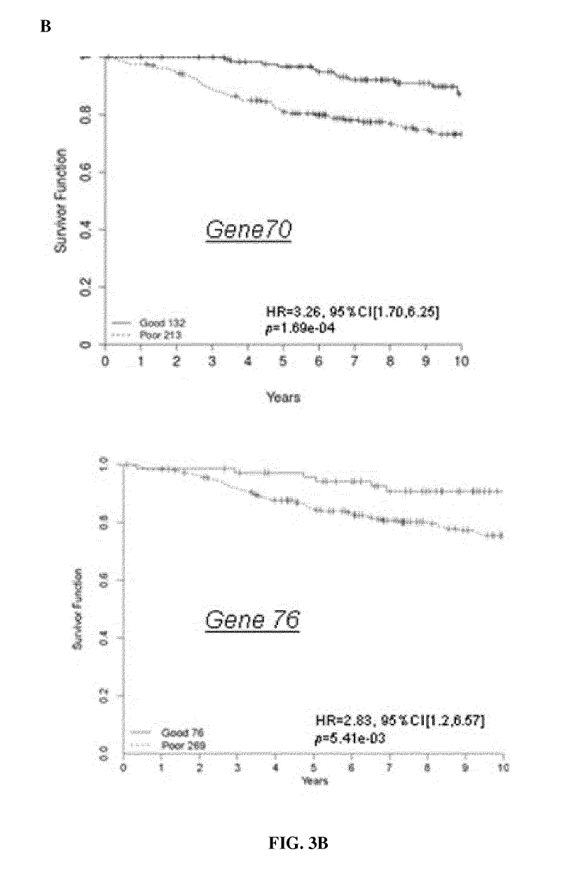

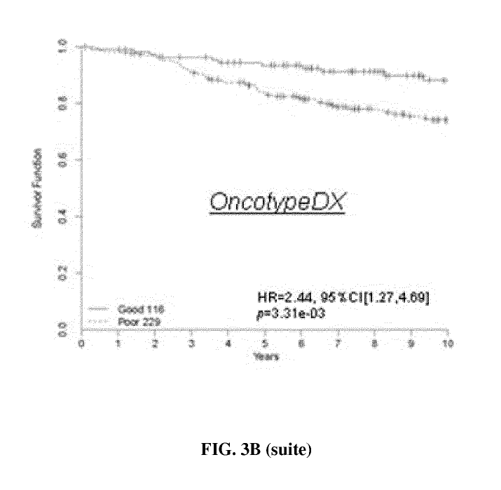

To evaluate the performance of the CycHyp signature, we compared it with other well-established prognostic multigene assays for breast cancer, namely Gene70 or Mammaprint (van't Veer et al, Nature 415:530-536, 2002), Gene76 (Wang et al, Lancet 365:671-679, 2005) and Oncotype Dx (Paik et al, N Engl J Med 351:2817-2826, 2004). Using the same set of ER+/HER2− node negative patients as used in FIG. 2D, we could determine the low vs. high risk patient stratification according to these signatures. The Balanced Classification Rate (BCR) represents the average between sensitivity and specificity to discriminate between patients with progressing disease vs. disease-free at 5 years. The BCR was significantly higher for the CycHyp signature than the three other multigene assays (FIG. 3A) (p-values=1.3e-4, 1.4e-21 and 6e-10 vs. Gene70, Gene76 and Oncotype DX, respectively). The sensitivity and the specificity of CycHyp were actually both above 70% while for each of the three other signatures, the specificity parameter was below 45% (FIG. 3A). The concordance index, which is the probability of a high risk patient to relapse before a low risk patient, was also higher with the CycHyp signature (FIG. 3A). The superior prognostic potential of the CycHyp signature could also be captured from the comparison of the Kaplan Meier curves obtained with the Gene 70, Gene76 and Oncotype DX signatures (HR in the 2-3 range) and that derived from the CycHyp signature (compare FIG. 3B with FIG. 2D).

The CycHyp Signature in Association with NPI Offers a Powerful Prognostic Tool

We then aimed to determine whether the CycHyp signature could improve the Nottingham Prognostic Index (NPI) for better predicting the survival of operable breast cancers.

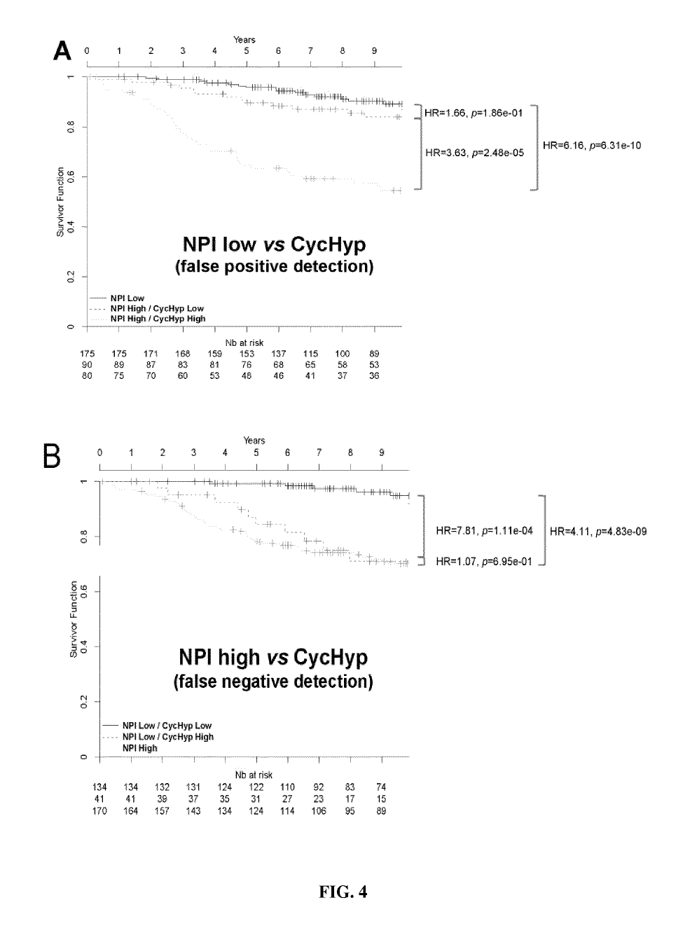

The NPI algorithm combines nodal status, tumour size and histological grade and allows modeling a continuum of clinical aggressiveness with 3 subsets of patients divided into good, moderate, and poor prognostic groups with 15-year survival (Rakha et al, Breas Cancer Res 12:207, 2010; Galea et al, Breast Cancer Res Treat 22:207-219, 1992; Balslev et al, Breast Cancer Res Treat 32:281-290, 1994). Since few patients were assigned a poor index, we merged here the moderate and poor indices into a high risk group to facilitate the comparison with the CycHyp signature. We found that by integrating the CycHyp signature, an important proportion of patients could be reclassified to another risk group (FIG. 4). 52.9% of patients classified at high risk using the NPI algorithm were “false positive” since identified at low risk when using the CycHyp signature and actually exhibited a profile of survival closer to the low risk NPI patient (FIG. 4A). Inversely, using the CycHyp signature, we also identified in the patients at low risk based on the NPI criteria, 23.4% of patients with a risk profile closer to the patients with a negative outcome (FIG. 4B).

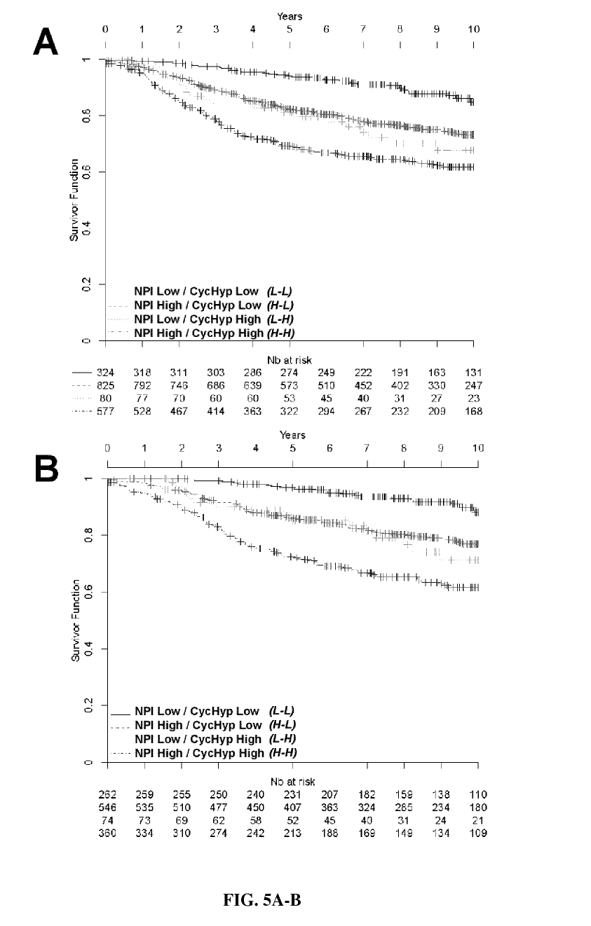

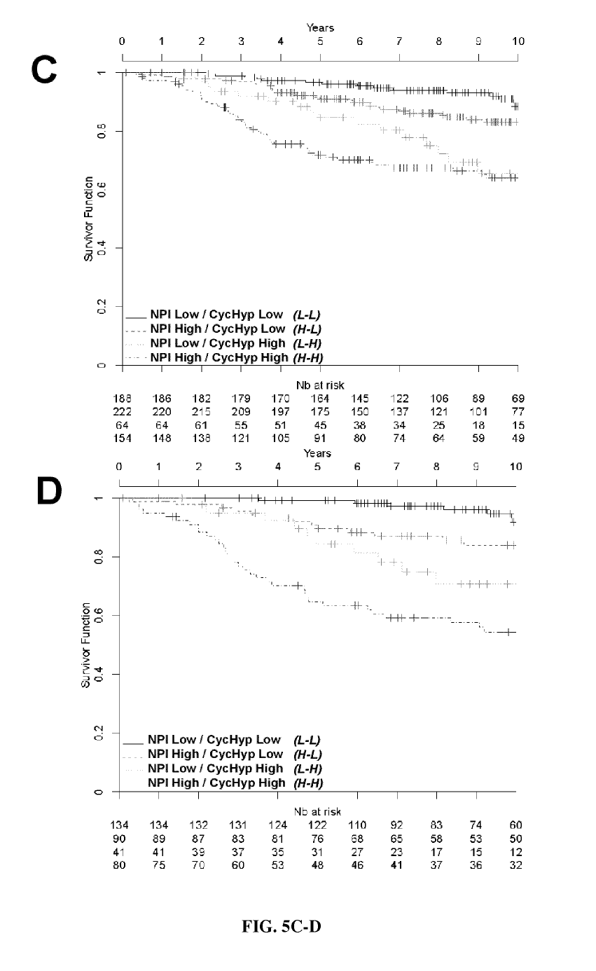

This increased discriminating potential remained highly relevant when considering all patients (FIG. 5A) or patients with a ER+HER2− status (FIG. 5B). Three subgroups of patients could be clearly identified: patients identified at low and high risks with both the CycHyp signature or the NPI criteria (Low-Low or High-High), and patients at intermediary risk, i.e. at low risk with NPI but high risk according to CycHyp signature (Low-High or false negative) or inversely (High-Low or false positive) (FIGS. 5A and 5B). Among the ER+HER2− patients with a node negative status (FIG. 5C), although a similar profiling of three subpopulations of patients could be proposed up to 5 years based on the combination of CycHyp signature and NPI, the gene signature was more efficient to predict survival on the longer term. Two subgroups of patients with either poor or good outcomes could actually be discriminated on the basis of the CycHyp signature (see L-L/H-L vs. L-H/H-H curves in FIG. 5C). Finally, when only considering untreated patients within the ER+HER2− node negative patients (FIG. 5D), four subgroups of patients were observed. Interestingly, within the subgroups of patients classified at intermediary risk, those at low risk based on the NPI but at high risk following the CycHyp (see L-H curve in FIG. 5D) had actually a worse outcome than those classified at high risk based on the NPI but at low risk according to the CycHyp signature (see red curve in FIG. 5D).

Numerical values obtained for patients and used for drafting FIGS. 5A-D are shown in the Table 13 below, wherein indicated p-values were derived from Mantel-Cox, log-rank tests.

TABLE 13

| | | L-L | | H-L | | L-H | |
|---|---|---|---|---|---|---|---|
| | | HR | p | HR | p | HR | p |
| All | H-L | 2.33 | 3.73e−07 | | | | |
| patients | L-H | 2.96 | 2.20e−04 | 1.22 | 0.392 | | |
| | H-H | 3.86 | 1.49e−17 | 1.69 | 3.04e−07 | 1.39 | 0.135 |
| ER+ HER2− | H-L | 2.72 | 3.45e−06 | | | | |
| (N+/N−) | L-H | 3.35 | 5.67e−04 | 1.17 | 0.569 | | |
| | H-H | 5.32 | 7.28e−17 | 1.97 | 3.63e−07 | 1.70 | 0.034 |
| ER+ HER2− | H-L | 2.11 | 1.89e−02 | | | | |
| (N−) | L-H | 4.41 | 9.83e−05 | 2.03 | 2.82e−02 | | |
| | H-H | 5.61 | 4.38e−10 | 2.72 | 9.12e−06 | 1.37 | 0.265 |
| ER+ HER2- | H-L | 3.93 | 5.42e−03 | | | | |
| (N−) | L-H | 7.81 | 1.11e−04 | 1.88 | 1.43e−01 | | |
| untreated | H-H | 14.34 | 9.17e−13 | 3.63 | 2.48e−05 | 1.98 | 0.044 |

Using the same protocol, the prognostic values of other signatures of the invention, comprising 10 probesets out of the 87 HGU133a probesets (themselves covering 69 genes of the CycHyp signature that are available on the HGU133a platform), were assessed.

The first 10-probesets signature comprises the following markers:

TABLE 14

| | Probeset | Probeset | GenBank | Name of the |
|---|---|---|---|---|
| | (HGU 1.0 ST) | (HGU133a) | Accession Number | marker |
| 1 | 8018860 | 202095_s_at | NM_001168 | BIRC5 |
| 2 | 8064156 | 221848_at | NM_032527 | ZGPAT |
| 3 | 8138912 | 202903_at | NM_012322 | LSM5 |
| 4 | 7921786 | 218336_at | NM_012394 | PFDN2 |
| 5 | 8165011 | 205237_at | NM_002003 | FCN1 |
| 6 | 7964262 | 200735_x_at | NM_001113201 | NACA |
| 7 | 7949792 | 204960_at | NM_005608 | PTPRCAP |
| 8 | 8034101 | 203679_at | NM_006858 | TMED1 |
| 9 | 8168087 | 202105_at | NM_001551 | IGBP1 |
| 10 | 7963575 | 211938_at | NM_001417 | EIF4B |

Probesets according to the HGU 1.0 ST platform and to the HGU133a platform are indicated.

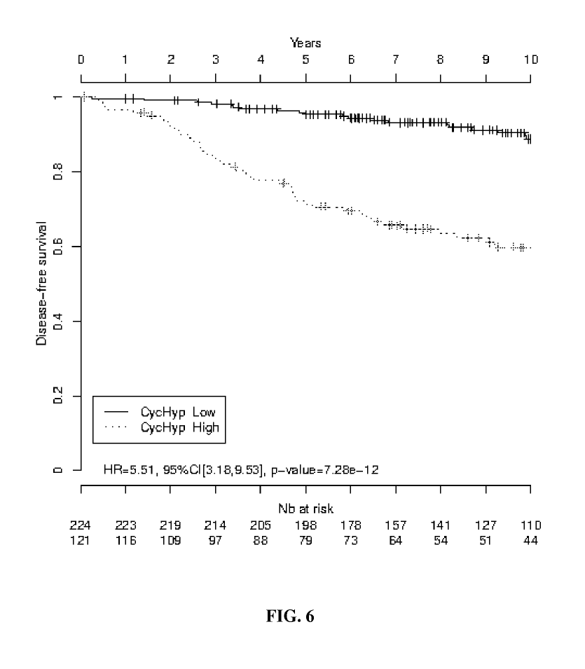

The prognostic efficiency of this signature is illustrated by the results of FIG. 6.

The second 10-probesets signature comprises the following markers:

TABLE 15

| | Probeset | GenBank | Name of the |
|---|---|---|---|
| | (HGU133a) | Accession Number | marker |
| 1 | 202094_at | AA648913 | BIRC5 |
| 2 | 210334_x_at | AB028869 | BIRC5 |
| 3 | 204249_s_at | NM_005574 | LMO2 |
| 4 | 209731_at | U79718 | NTHL1 |
| 5 | 200018_at | NM_001017 | RPS13 |
| 6 | 218391_at | NM_007241 | SNF8 |
| 7 | 202904_s_at | NM_012322 | LSM5 |
| 8 | 208635_x_at | BF976260 | NACA |
| 9 | 211747_s_at | BC005938 | LSM5 |
| 10 | 208904_s_at | BC000354 | RPS28 |

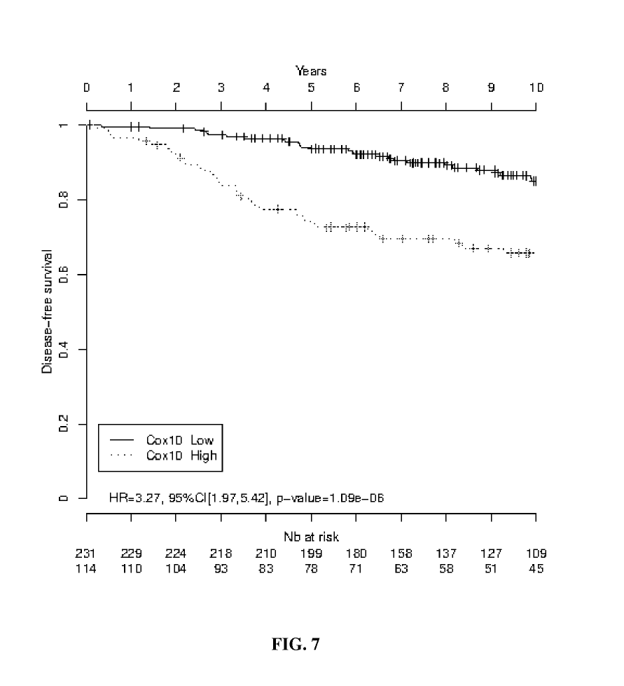

The prognostic efficiency of this signature is illustrated by the results of FIG. 7.

The third 10-probesets signature comprises the following markers:

TABLE 16

| | Probeset | GenBank | Name of the |
|---|---|---|---|
| | (HGU133a) | Accession Number | marker |
| 1 | 202095_s_at | NM_001168 | BIRC5 |
| 2 | 221434_s_at | NM_031210 | C14orf156 |
| 3 | 202904_s_at | NM_012322 | LSM5 |
| 4 | 33 211747_s_at | BC005938 | LSM5 |
| 5 | 200703_at | NM_003746 | DYNLL1 |
| 6 | 218391_at | NM_007241 | SNF8 |
| 7 | 208903_at | BF431363 | RPS28 |
| 8 | 200018_at | NM_001017 | RPS13 |
| 9 | 208635_x_at | BF976260 | NACA |
| 10 | 218177_at | AA293502 | CHMP1B |

The prognostic efficiency of this signature is illustrated by the results of FIG. 8.

The fourth 10-probesets signature comprises the following markers:

TABLE 17

| | Probeset | GenBank | Name of the |
|---|---|---|---|
| | (HGU133a) | Accession Number | marker |
| 1 | 202095_s_at | NM_001168 | BIRC5 |
| 2 | 211938_at | BF247371 | EIF4B |
| 3 | 221434_s_at | NM_031210 | C14orf156 |
| 4 | 202904_s_at | NM_012322 | LSM5 |
| 5 | 33 211747_s_at | BC005938 | LSM5 |
| 6 | 200703_at | NM_003746 | DYNLL1 |
| 7 | 218391_at | NM_007241 | SNF8 |
| 8 | 208903_at | BF431363 | RPS28 |
| 9 | 200018_at | NM_001017 | RPS13 |
| 10 | 208635_x_at | BF976260 | NACA |

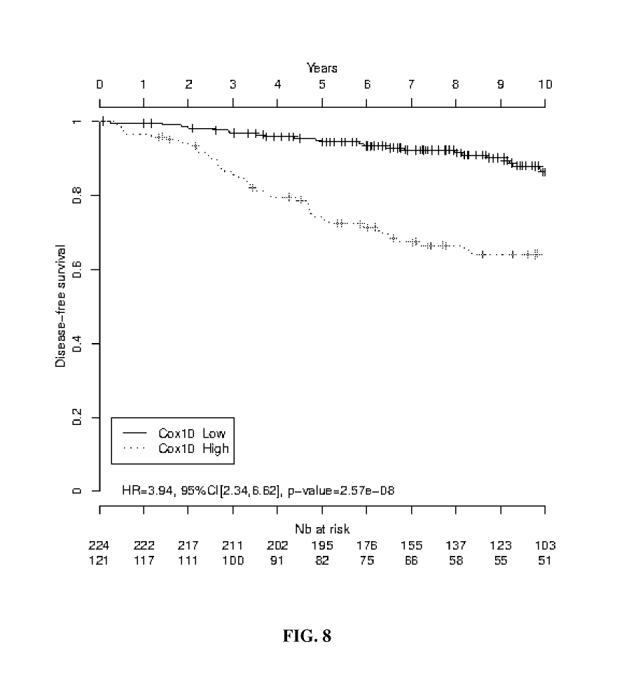

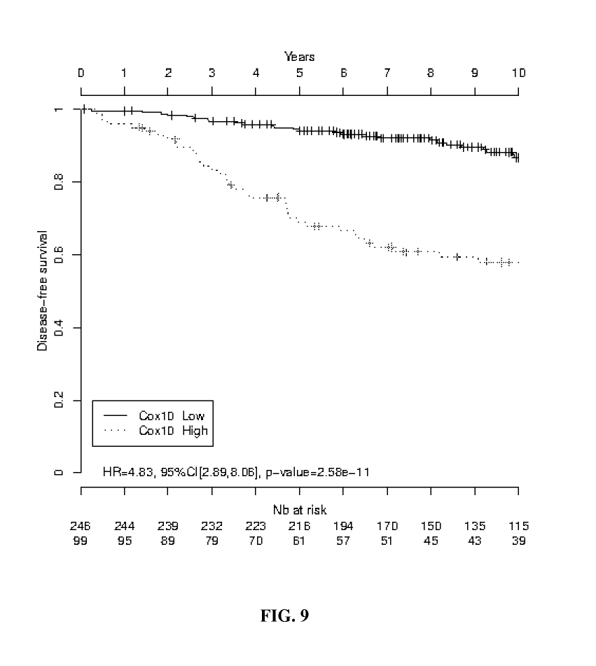

The prognostic efficiency of this signature is illustrated by the results of FIG. 9.

Taken together, these data demonstrate that the signatures of the present invention, which are derived from the transcriptomic adaptation of tumor cells to cycling hypoxia is prognostic of cancer.

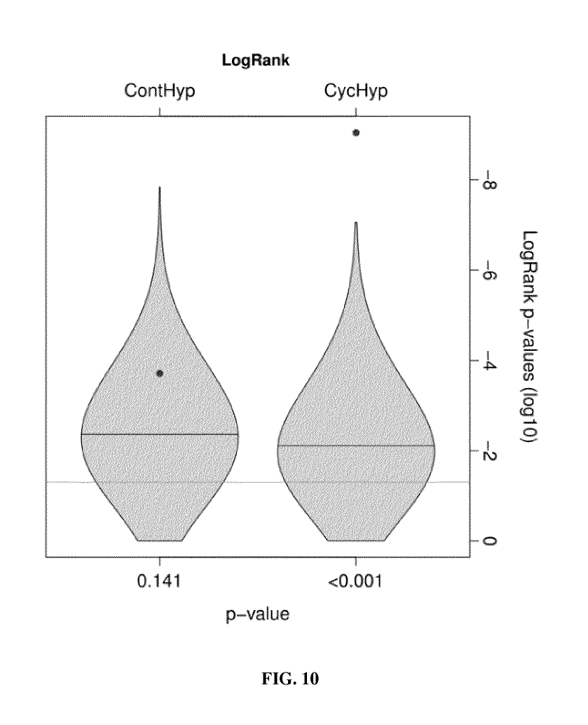

To confirm the specificity of these results, random gene signatures were tested for their prognostic capacity (negative control). These random signatures were constituted of 10 genes randomly selected amongst the totality of the genome. To have a significant value, 1000 such random signatures were used according the same methodology than with the CycHyp signature. The logrank test (or Mantel-Haenszel test; Balsev et al, Breast Cancer Res Treat, 1994) is commonly used to assess whether there is a significant survival difference between risk groups. The discrimination between risk groups was significantly higher (P<0.001) with the CycHyp signature as compared to each of the random signatures, therefore validating the prognostic potential of the CycHyp signature (right panel, FIG. 10).

### Example 2

### Alternative Lists of 10 Genes

### Methods

To assess the prognosis value of an alternative list of 10 genes representative of Cycling Hypoxia, we compared the CycHyp signature with alternative lists of 10 probesets (Table 18) out of the 87 HGU133a probesets (themselves covering 69 genes of the CycHyp signature that are available on the HGU133a platform but without overlap with the CycHyp signature of 10 genes shown in Table 8. Using the same set of ER+/HER2-node negative patients as used in FIG. 2D, we could determine the low vs. high risk patient stratification according to these signatures.

TABLE 18

| | Probeset | GenBank | Name of the |
|---|---|---|---|
| | (HGU133a) | Accession number | marker |
| 1 | 204249_s_at | NM_005574 | LMO2 |
| 2 | 209731_at | U79718 | NTHL1 |
| 3 | 200018_at | NM_001017 | RPS13 |
| 4 | 218391_at | NM_007241 | SNF8 |
| 5 | 208904_s_at | BC000354 | RPS28 |
| 6 | 222216_s_at | AK026857 | MRPL17 |
| 7 | 201758_at | NM_006292 | TSG101 |
| 8 | 200703_at | NM_003746 | DYNLL1 |
| 9 | 209467_s_at | BC002755 | MKNK1 |
| 10 | 208903_at | BF431363 | RPS28 |

### Results

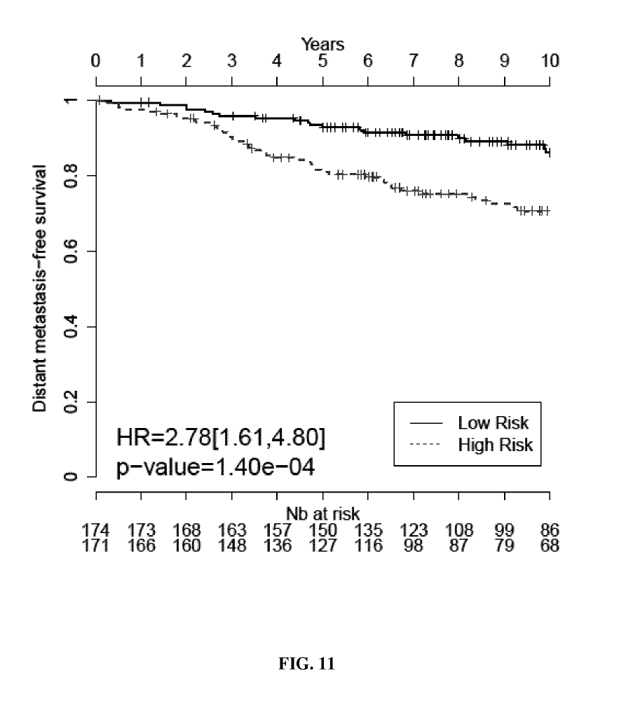

The prognostic efficiency of one of these alternative signatures is illustrated by the results of FIG. 11. Such a model may have a good prognosis performance with a hazard ratio of 2.78 for that particular alternative list, but is significantly lesser than the CycHyp model (HR=5.51, see FIG. 2D).

Another 10-probesets signature wherein one probeset of Table 18 is replaced by one probeset of Table 14 comprises the following markers:

TABLE 19

| | Probeset | GenBank | Name of the |
|---|---|---|---|
| | (HGU133a) | Accession number | marker |
| 1 | 211938_at | BF247371 | EIF4B |
| 2 | 204249_s_at | NM_005574 | LMO2 |
| 3 | 209731_at | U79718 | NTHL1 |
| 4 | 200018_at | NM_001017 | RPS13 |
| 5 | 218391_at | NM_007241 | SNF8 |
| 6 | 208904_s_at | BC000354 | RPS28 |
| 7 | 222216_s_at | AK026857 | MRPL17 |
| 8 | 201758_at | NM_006292 | TSG101 |
| 9 | 200703_at | NM_003746 | DYNLL1 |
| 10 | 209467_s_at | BC002755 | MKNK1 |

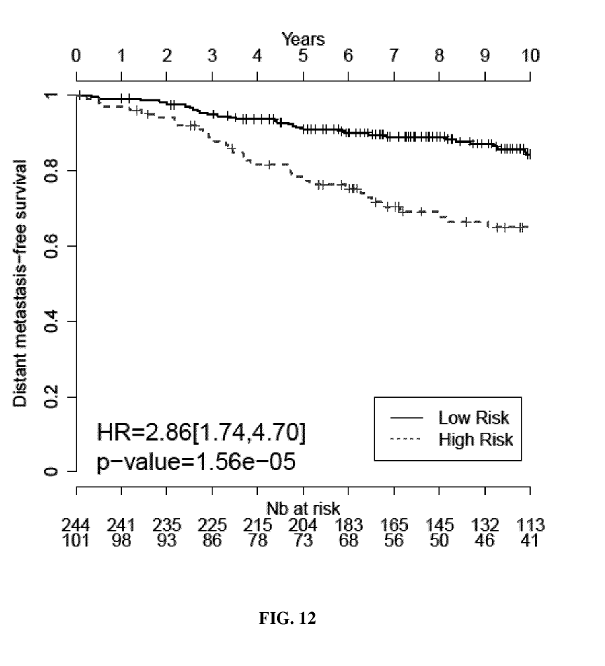

The prognostic efficiency of this signature is illustrated by the results of FIG. 12.

Another 10-probesets signature where two probesets of Table 18 are replaced by two probesets of Table 14 comprises the following markers:

TABLE 20

| | Probeset | GenBank | Name of the |
|---|---|---|---|
| | (HGU133a) | Accession number | marker |
| 1 | 202095_s_at | NM_001168 | BIRC5 |
| 2 | 211938_at | BF247371 | EIF4B |
| 3 | 204249_s_at | NM_005574 | LMO2 |
| 4 | 209731_at | U79718 | NTHL1 |
| 5 | 200018_at | NM_001017 | RPS13 |
| 6 | 218391_at | NM_007241 | SNF8 |
| 7 | 208904_s_at | BC000354 | RPS28 |
| 8 | 222216_s_at | AK026857 | MRPL17 |
| 9 | 201758_at | NM_006292 | TSG101 |
| 10 | 200703_at | NM_003746 | DYNLL1 |

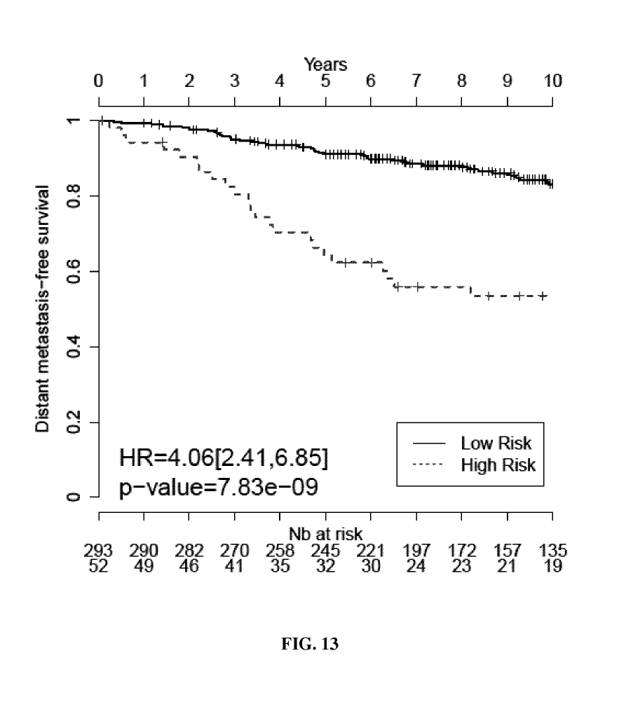

The prognostic efficiency of this signature is illustrated by the results of FIG. 13.

Equivalent results were obtained with the other alternative lists tested. These results thus demonstrated that any combination of 10 genes of Table 5 or Table 11 has a high prognosis performance.

### Example 3

### Comparison of the Signature of Cyclic Hypoxia of the Invention (CycHyp) with a Signature of Continuous Hypoxia (ContHyp)

### Methods

Using the same protocol as for the identification of the CycHyp signature, we determined a ContHyp signature which corresponds to continuous hypoxia conditions, i.e. 24 h continuous exposure to 1% O₂.

### Results

A heatmap made with the 100 probe sets of the CycHyp signature shown its important potential of discrimination between cycling hypoxia and continuous hypoxia (data not shown).

We then used the Gene Set Enrichment Analysis described by Subramanian et al. (Proc Natl Acad Sci USA, 2005) which is a method for identifying differentially expressed genes that share some characteristic. The analysis indicated that when considering differentially expressed probesets (after FDR correction), only 2 gene sets were significantly enriched in the CycHyp signature whereas 52 gene sets were enriched in the ContHyp signature, including 17 directly related to hypoxia.

Also, when using the MSigDB molecular signature database referring to hypoxia or HIF (www.broadinstitute.org), we found only 13 hypoxia gene sets sharing, on average, 1.4 gene with CycHyp whereas 44 hypoxia gene sets showed overlap with ContHyp with an average of 6.6 common genes.

To further validate the prognosis significance of the CycHyp signature compared to the ContHyp signature, we performed a comparison with random gene signatures according to the methodology described by Venet et al. (PLoS Comput Biol, 2011) and Beck et al. (PLoS Comput Biol, 2013). FIG. 10 shows the distribution of the p-values (log rank test in log 10) for 1000 randomly generated signatures together with the p-values of the CycHyp and ContHyp signatures. The discrimination between risk groups was significantly higher (P<0.001) with the CycHyp signature as compared to each of the random signatures whereas the ContHyp signature (left panel) was not significantly better (vs. random ones; P=0.141).

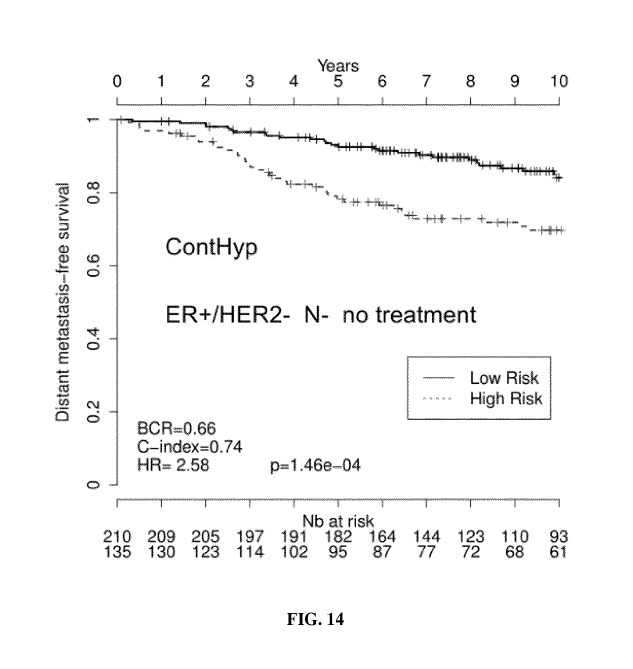

Using the same methodology, we examined the prognostic capacity of the ContHyp signature (discriminating between normoxia and continuous hypoxia). The performance of the ContHyp signature was satisfactory on the ER+HER2− untreated population (HR=2.58, p-value=1.46e-4, see FIG. 14) but was significantly lower (p-value=3.61e-8) than the CycHyp signature.

Taken together, these data confirm the significantly high value of the CycHyp signature of the present invention, and confirm the prognostic advantage of a signature based on cyclic hypoxia compared to a signature based on continuous hypoxia.

### Example 4

### CycHyp Signature on Colorectal Cancer Patients

### Patients and Methods

To validate the use of the CycHyp signature on colorectal cancer, we used 2 public microarray data sets: GSE39582 (566 patients) and GSE17536 (177 patients).

The GSE39582 dataset was used as the training set used to estimate a prognostic model of the clinical outcome. This training dataset was used to estimate a prognostic Cox or equal weights linear (EWL) regression models built on the CycHyp signature. The GSE17536 dataset was then used according to the methodology described for breast cancer samples to assess the prognostic performance of the CycHyp signature on independent samples.

### Results

As for breast cancer, we first compared the CycHyp signature with randomly selected genes on the colon data sets. Each random signature has the same size as the CycHyp signature. We generated 1,000 such random signatures and use the same methodology to estimate a prognosis model from the GSE39582 dataset. We then assess the performance of those prognosis models on the independent validation sets of 177 patients (GSE17536).

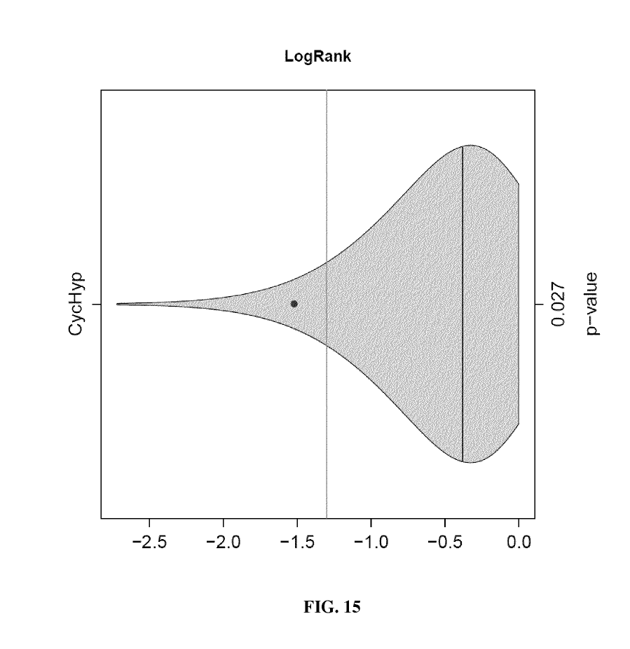

FIG. 15 represents in gray the distribution of those prognosis models built from random signatures on the stage 2 patients. The discrimination between high and low risk groups is assessed according to a logrank test and its associated p-value (reported in log 10 scale). The logrank p-values of the CycHyp signature is represented with black dots on the same plot. The CycHyp signature is significantly better than random signature on the stage 2 patients (p-value 0:027).

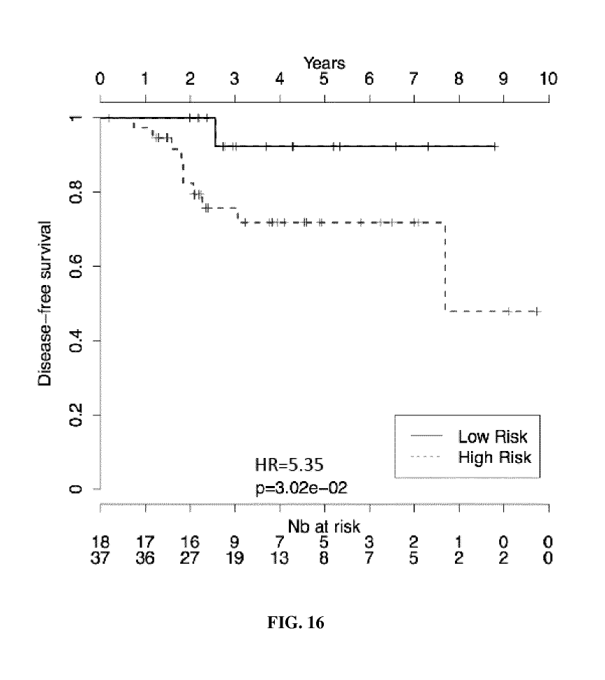

To evaluate the discriminating capacity of the CycHyp signature, we chose to focus on the stage II colorectal cancer population which is known to be heterogeneous and thus difficult to treat. The prognostic efficiency of the CycHyp signature is illustrated by the results of FIG. 16. The discriminating capacity of the CycHyp signature was strikingly high in this patient population (HR=5.35, p-value=0.03) when compared with the whole colorectal cancer patient population (HR=2.52, p-value=0.017) (not shown).

These results demonstrate that the CycHyp signature of the invention also has high prognosis performance for colorectal cancer.

### Example 5

### Prognostic Performance of a Signature Consisting of BIRC5, IGBP1 and EIF4B

### Methods

The prognostic values of another signature of the invention, comprising 3 probesets, was assessed. Using the same set of ER+/HER2− node negative patients as used in FIG. 2D, we could determine the low vs. high risk patient stratification according to these signatures.

The 3-probesets signature comprises the following markers:

TABLE 21

| | Probeset | GenBank | Name of the |
|---|---|---|---|
| | (HGU133a) | Accession number | marker |
| 1 | 202095_s_at | NM_001168 | BIRC5 |
| 2 | 202105_at | NM_001551 | IGBP1 |
| 3 | 211938_at | BF247371 | EIF4B |

### Results

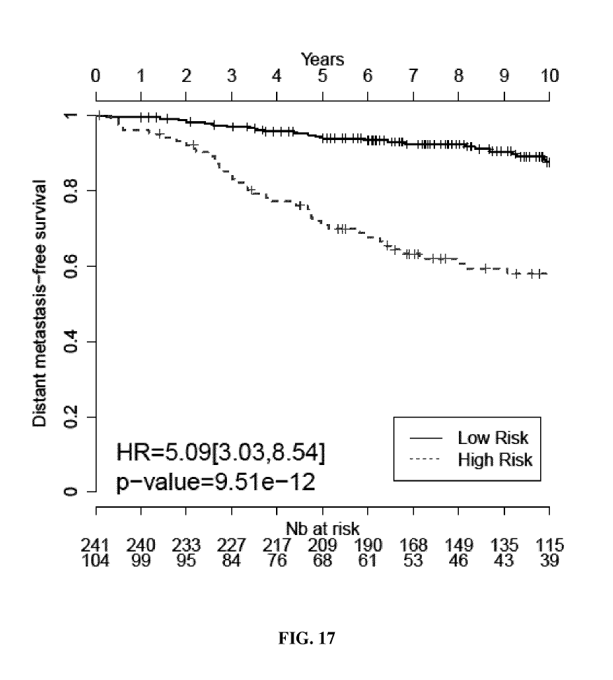

The prognostic efficiency of this signature is illustrated by the results of FIG. 17. Such a model has a good prognosis performance with a hazard ratio of 5.09 that is almost as good as the CycHyp model (HR=5.51, see FIG. 2D).

1. A non-invasive method for the prognosis of cancer in a subject, or for predicting the response of a subject to a specific treatment, wherein said method comprises obtaining a sample from the subject, and
assessing the expression of markers of a signature comprising at least 2 cycling hypoxia markers.

2. The non-invasive method according to claim 1, wherein said signature comprises at least 3 cycling hypoxia markers.

3. The non-invasive method according to claim 1, wherein said cycling hypoxia markers are selected from the list of 1379 cycling hypoxia markers of Table 1, fragments, variants and equivalents thereof.

4. The non-invasive method according to claim 1, wherein said cycling hypoxia markers are selected from the list of 651 cycling hypoxia markers of Table 2, fragments, variants and equivalents thereof.

5. The non-invasive method according to claim 1, wherein said cycling hypoxia markers are selected from the list of 298 cycling hypoxia markers of Table 3, fragments, variants and equivalents thereof.

6. The non-invasive method according to claim 1, wherein said cycling hypoxia markers are selected from the list of 167 cycling hypoxia markers of Table 4, fragments, variants and equivalents thereof.

7. The non-invasive method according to claim 1, wherein said cycling hypoxia markers are selected from the list of 96 cycling hypoxia markers of Table 5, fragments, variants and equivalents thereof.

8. The non-invasive method according to claim 1, wherein said cycling hypoxia markers are selected from the list of 74 cycling hypoxia markers of Table 6, fragments, variants and equivalents thereof.

9. The non-invasive method according to claim 1, wherein said cycling hypoxia markers are selected from the list of 37 cycling hypoxia markers of Table 7, fragments, variants and equivalents thereof.

10. The non-invasive method according to claim 1, wherein said cycling hypoxia markers are selected from the list of 10 cycling hypoxia markers of Table 8, fragments, variants and equivalents thereof.

11. The non-invasive method according to claim 1, wherein said signature comprises the 10 cycling hypoxia markers of Table 8, variants, fragments and equivalents thereof.

12. The non-invasive method according to claim 1, wherein said method comprises mathematically combining the expression profile of markers in a score.

13. The non-invasive method according to claim 1, wherein said sample is a biopsy sample or a bodily fluid sample of said subject.

14. The non-invasive method according to claim 1, further comprising comparing said expression with a reference expression profile.

15. A kit for implementing the non-invasive method according to claim 1, wherein said kit comprises means for determining the expression of the cycling hypoxia markers of the signature.

16. The kit according to claim 15, wherein said means for determining the expression of the markers of the signature is a microarray comprising probes specific for said cycling hypoxia markers.

17. The kit according to claim 15, wherein said means for determining the expression of the cycling hypoxia markers are qPCR primers specific for said cycling hypoxia markers.

18. The kit according to claim 15, wherein said means for determining the expression of the cycling hypoxia markers are probes to detect qPCR amplicons obtained with qPCR primers.


SEQUENCE LISTING (9 sequences)

SEQ ID NO: 1 - DNA, 20 bp, Artificial Sequence
  Synthetic forward primer BIRC5
agggctgaagtctggcgtaa

SEQ ID NO: 2 - DNA, 22 bp, Artificial Sequence
  Synthetic reverse primer BIRC5
aacaatccaccctgcagctcta

SEQ ID NO: 3 - DNA, 18 bp, Artificial Sequence
  Synthetic probe BIRC5
atgatggatttgattcgc

SEQ ID NO: 4 - DNA, 21 bp, Artificial Sequence
  Synthetic forward primer NACA
ccacccctaaatctgctggaa

SEQ ID NO: 5 - DNA, 22 bp, Artificial Sequence
  Synthetic reverse primer NACA
tccagaccccttgttgttcttc

SEQ ID NO: 6 - DNA, 14 bp, Artificial Sequence
  Synthetic probe NACA
ccctgtcccaaccc

SEQ ID NO: 7 - DNA, 17 bp, Artificial Sequence
  Synthetic forward primer IGBP1
gtccgcgctcgcctaat

SEQ ID NO: 8 - DNA, 22 bp, Artificial Sequence
  Synthetic reverse primer IGBP1
gagagaggaacccggaagatct

SEQ ID NO: 9 - DNA, 20 bp, Artificial Sequence
  Synthetic probe IGBP1
ctttatcaaggttgcctttg